(19) United States
(12) Patent Application Publication
(10) Pub. No.: US 2024/0398822 A1
(43) Pub. Date: Dec. 5, 2024

(54) METHODS AND COMPOSITIONS FOR TREATING AND MANAGING ALZHEIMER'S DISEASE

(71) Applicant: Pharmatrophix, Inc., Menlo Park, CA (US)

(72) Inventor: Frank M. LONGO, Menlo Park, CA (US)

(21) Appl. No.: 18/537,611

(22) Filed: Dec. 12, 2023

Related U.S. Application Data
(63) Continuation of application No. PCT/US22/25127, filed on Apr. 15, 2022.
(60) Provisional application No. 63/212,050, filed on Jun. 17, 2021.
(60) Provisional application No. 63/213,169, filed on Jun. 21, 2021.
(60) Provisional application No. 63/229,948, filed on Aug. 5, 2021.
(60) Provisional application No. 63/275,234, filed on Nov. 3, 2021.
(60) Provisional application No. 63/277,691, filed on Nov. 10, 2021.
(60) Provisional application No. 63/296,678, filed on Jan. 5, 2022.
(60) Provisional application No. 63/306,425, filed on Feb. 3, 2022.
(60) Provisional application No. 63/311,780, filed on Feb. 18, 2022.

(51) Int. Cl. A61K 31/5375; A61P 25/28

(52) U.S. Cl. CPC A61K 31/5375; A61P 25/28

(57) ABSTRACT

The disclosure provides compounds, salts and methods of use thereof for the prevention, treatment, delay of onset and management of Alzheimer's disease.

### CROSS REFERENCE

This application is a continuation of International Patent Application No. PCT/US2022/025127 filed Apr. 15, 2022, which claims the benefits of U.S. Provisional Application No. 63/212,050 filed Jun. 17, 2021; U.S. Provisional Application No. 63/213,169 filed Jun. 21, 2021; U.S. Provisional Application No. 63/229,948 filed Aug. 5, 2021; U.S. Provisional Application No. 63/275,234 filed Nov. 3, 2021; U.S. Provisional Application No. 63/277,691 filed Nov. 10, 2021; U.S. Provisional Application No. 63/296,678 filed Jan. 5, 2022; U.S. Provisional Application No. 63/306,425 filed Feb. 3, 2022; and U.S. Provisional Application No. 63/311,780 filed Feb. 18, 2022; each of which is incorporated herein by reference in its entirety for all purposes.

### STATEMENT REGARDING FEDERALLY SPONSORED RESEARCH OR DEVELOPMENT

This invention was made with Government support under NIA AD Pilot Trial 1RO1AG051596, awarded by the National Institute of Health (NIH). The Government has certain rights in this invention.

### BACKGROUND

According to the Alzheimer's Association, some 5.4 million individuals in the United States (US) have Alzheimer's disease (AD) and more than 95% of these are aged 65 years or older. One in 8 Americans age 65 and older, and almost one-half of those age 85 and older have AD. In addition to the incalculable human costs for individuals and their families, the estimated amount that was paid out to healthcare providers, nursing homes and hospice for Alzheimer's care in 2015 was $226 billion. With aging demographics, the number of affected individuals and the cost of care are in the early stages of an exponential rise with costs expected to reach $1.1 trillion by 2050.

Among the top 10 causes of death in the US, AD is the only cause that has no treatment options proven to prevent, delay, cure, or even slow its progression. Approved treatments are limited to amelioration of cognitive symptoms without having significant effects on underlying neurodegeneration and the disease process. The first approved class of symptomatic treatments consists of the acetylcholinesterase inhibitors (donepezil, rivastigmine, and galantamine) with the most recent FDA approval in this class occurring in 2001. A second class of symptomatic treatment was defined by the drug memantine, an N-methyl-D-aspartate receptor antagonist, approved by the FDA in 2003. These drugs have, at best, modest effects on cognition, overall function, and caregiver ratings, and no clearly detectable effects on AD progression. The goal of achieving a therapy that slows the underlying progression of neuronal degeneration has largely focused on efforts to reduce the accumulation of amyloid beta (Aβ) either by inhibition of enzymes necessary for Aβ production or by immunization against the Aβ peptide. Enzyme inhibition has been limited by toxic effects and recent phase 3 results from immunization approaches have so far been disappointing. The first, and so far only, amyloid antibody obtaining FDA approval (2021) is aducanumab. The extent to which aducanumab has a clinical effect in slowing disease progression is a controversial topic. It is clear that aducanumab confers considerable risk for brain edema and brain hemorrhage as side effects. New therapeutic strategies are needed to address this debilitating disease.

Primary features of AD include the accumulation of amyloid plagues and tau protein tangles along with failure of synaptic function, the degeneration of neuronal synapses, gliosis/neuro-inflammation. The p75 neurotrophin receptor has theoretical and complex links to many of these processes with potentially positive or negative affects and it is not known if its modulation could affect any aspect of AD or its fluid or imaging biomarkers.

### SUMMARY

The disclosure provides compounds, salts and methods of use thereof for the prevention, delay of onset, treatment, slowing progression of and management of Alzheimer's disease. This strategy is focused on modulation of the p75 neurotrophin receptor. Prior to the present disclosure, it has not been known whether targeting or modulation of this receptor could slow progression of markers of AD or affect markers of any human disease state. The present formulation is a first-in-class modulator of the p75 neurotrophin receptor and the present disclosure in the first experience in the application of p75 receptor modulation in a human disease state.

In some embodiments, the disclosure of the present application provides a method for modulating one or more neurodegeneration biomarkers or indicators in a subject in need thereof, the method comprising: administering to said subject an effective amount of a p75^{NTR} receptor modulator or a pharmaceutically acceptable salt thereof to provide one or more of the following in said subject: (i) an amyloid beta (Aβ) level (e.g., in a bodily fluid) that is lower than a corresponding reference; (ii) a change (e.g., increase) or a rate of change (e.g., a rate of increase) in an Aβ level (e.g., in a bodily fluid) over a duration that is lower than a corresponding reference over the same duration; (iii) a change (e.g., increase) or a rate of change (e.g., a rate of increase) in a tau (i) level (e.g., in a bodily fluid) over a duration that is lower than a corresponding reference over the same duration; (iv) a change (e.g., increase) or a rate of change (e.g., a rate of increase) in a presynaptic biomarker level (e.g., in a bodily fluid) over a duration that is lower than a corresponding reference over the same duration; (v) a change (e.g., increase) or a rate of change (e.g., a rate of increase) in a postsynaptic biomarker level (e.g., in a bodily fluid) over a duration that is lower than a corresponding reference over the same duration; (vi) a change (e.g., increase) or a rate of change (e.g., a rate of increase) in a glial marker level (e.g., in a bodily fluid) over a duration that is lower than a corresponding reference over the same duration; (vii) a rate of volume change of a brain region over a duration that is lower than a corresponding reference rate (e.g., in an untreated control) over the same duration as determined by magnetic resonance imaging (MRI) imaging; (viii) a brain glucose metabolism (¹⁸F-FDG-PET) rate that is lower than a corresponding reference rate (e.g., in an untreated control); and (ix) a change in Alzheimer's Disease Assessment Scale-Cognitive (ADAS-Cog) score over a duration that is less than a corresponding change (e.g., in an untreated control) over the same duration.

In some aspects, the disclosure provides for a method for preventing, treating, ameliorating, managing, delaying onset, or slowing progression of Alzheimer's disease in a subject in need thereof, comprising administering to the subject a therapeutically effective amount of a pharmaceutical composition comprising a salt of a compound represented by the formula,

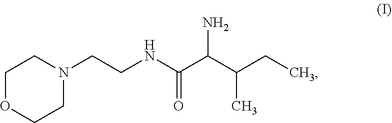 [SMILES: CCC(C)C(N)C(=O)NCCN1CCOCC1]

thereby preventing, treating, ameliorating, managing, delaying onset, or slowing progression of Alzheimer's disease in the subject. In some embodiments, the salt of the compound of Formula (I) is represented by Formula (II):

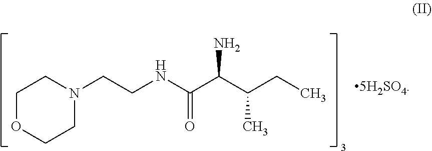

In some embodiments, the pharmaceutical composition comprises from about 10 milligram (mg) (free base weight) to about 1000 mg (free base weight) of the salt of the compound of Formula (I). In some embodiments, the pharmaceutical composition comprises from about 10 milligram (mg) (free base weight) to about 900 mg (free base weight) of the salt of the compound of Formula (I). In some embodiments, the pharmaceutical composition comprises from about 10 milligram (mg) (free base weight) to about 800 mg (free base weight) of the salt of the compound of Formula (I). In some embodiments, the pharmaceutical composition comprises from about 10 milligram (mg) (free base weight) to about 600 mg (free base weight) of the salt of the compound of Formula (I). In some embodiments, the pharmaceutical composition comprises from about 10 milligram (mg) (free base weight) to about 500 mg (free base weight) of the salt of the compound of Formula (I). In some embodiments, the pharmaceutical composition comprises from about 10 milligram (mg) (free base weight) to about 400 mg (free base weight) of the salt of the compound of Formula (I). In some embodiments, the pharmaceutical composition comprises from about 10 milligram (mg) (free base weight) to about 300 mg (free base weight) of the salt of the compound of Formula (I). In some embodiments, the pharmaceutical composition comprises from about 10 milligram (mg) (free base weight) to about 200 mg (free base weight) of the salt of the compound of Formula (I). In some embodiments, the pharmaceutical composition comprises from about 10 milligram (mg) (free base weight) to about 100 mg (free base weight) of the salt of the compound of Formula (I). In some embodiments, the composition is in the form of a tablet. In some embodiments, the composition is in a capsule. In some embodiments, the composition is in the form of a pill. In some embodiments, the pharmaceutical composition is administered more than once daily. In some embodiments, the pharmaceutical composition is administered twice daily. In some embodiments, the pharmaceutical composition is administered three times daily. In some embodiments, the administering is orally. In some embodiments, the p75NTR receptor modulator or the pharmaceutically acceptable salt thereof, or the pharmaceutical composition, is formulated for administration at a dose of 100 mg b.i.d. (bis in die, twice a day). In some embodiments, the p75NTR receptor modulator or the pharmaceutically acceptable salt thereof, or the pharmaceutical composition, is formulated for administration at a dose of 200 mg b.i.d. In some embodiments, the p75NTR receptor modulator or the pharmaceutically acceptable salt thereof, or the pharmaceutical composition, is formulated for administration at a dose of 300 mg b.i.d. In some embodiments, the p75NTR receptor modulator or the pharmaceutically acceptable salt thereof, or the pharmaceutical composition, is formulated for administration at a dose of 400 mg b.i.d. In some embodiments, the p75NTR receptor modulator or the pharmaceutically acceptable salt thereof, or the pharmaceutical composition, is formulated for administration at a dose of 100 mg b.i.d. to 800 mg b.i.d. In some embodiments, the p75NTR receptor modulator or the pharmaceutically acceptable salt thereof, or the pharmaceutical composition, is formulated for administration at a dose of 100 mg b.i.d. to 700 mg b.i.d. In some embodiments, the p75NTR receptor modulator or the pharmaceutically acceptable salt thereof, or the pharmaceutical composition, is formulated for administration at a dose of 100 mg b.i.d. to 600 mg b.i.d. In some embodiments, the p75NTR receptor modulator or the pharmaceutically acceptable salt thereof, or the pharmaceutical composition, is formulated for administration at a dose of 100 mg b.i.d. to 500 mg b.i.d. In some embodiments, the p75NTR receptor modulator or the pharmaceutically acceptable salt thereof, or the pharmaceutical composition, is formulated for administration at a dose of 100 mg b.i.d. to 400 mg b.i.d. In some embodiments, the p75NTR receptor modulator or the pharmaceutically acceptable salt thereof, or the pharmaceutical composition, is formulated for administration at a dose of 100 mg b.i.d. to 300 mg b.i.d. In some embodiments, the subject exhibits or is determined to exhibit mild to moderate Alzheimer's disease according to McKhann (2011) criteria within six months prior to the administration. In some embodiments, the subject exhibits or is determined to be in earlier stages of clinical or preclinical Alzheimer's disease. In some embodiments, the subject exhibits or is determined to exhibit an ε4 allele of apolipoprotein E (ApoE) gene. In some embodiments, the subject exhibits or is determined to not exhibit an E4 allele of apolipoprotein E (ApoE) gene. In some embodiments, the subject is between the ages of 50 and 90. In some embodiments, subjects might exhibit none or one or more imaging, CSF, blood, plasma or other biomarkers consistent with a underlying status of Alzheimer's disease pathology such as a positive PET scan for amyloid or pathological tau; blood or plasma measurements with abnormal levels of markers such as p-tau, Ab-42; or CSF with abnormal levels or ratios of biomarkers such as Ab 42 or 40, p-tau or tau. In some embodiments, the method results in an improvement of, or mitigates or prevents a deterioration of, a value of one or more Alzheimer's disease metrics relative to a baseline value measured within six months (e.g., within three months) or after six months (e.g. at 12 or 18 months) preceding the administration. In some embodiments, the Alzheimer's disease metrics are selected from anatomical or statistical regional or voxel-based brain glucose metabolism (¹⁸F-FDG-PET) rate which is a surrogate measure of synaptic function, magnetic resonance imaging (MRI) structural or volumetric or voxel based imaging which is a measure of neuronal or synaptic degeneration, cerebrospinal Alzheimer's and neurodegenerative disease-related, or inflammatory or gliosis biomarker levels, and performance on the cognitive testing using standard methods such as the ADAS-cog, mini mental status exam (MMSE) and other formats. In some embodiments, the cerebrospinal and/or blood or plasma Alzheimer's disease-related biomarker levels include the levels of one or more of tau, p-tau, Aβ40, Aβ42, AchE, neurofilament light chain, SNAP-25, neurogranin, synaptotagmin-1, sTREM and YKL-40. In some embodiments, the cognitive testing methods include one or more of the Alzheimer's disease assessment scale (ADAS-Cog), the mini mental status exam (MMSE), Clinical Global Impression (CGI) scale, and Geriatric Depression Scale (GDS). In some embodiments, cognitive testing methods include the neurological testing battery (NTB) or various combinations of its elements. In some embodiments, the method reduces or prevents loss of hippocampus volume or loss or change of volume in other brain areas or voxel-based brain regions indicative of neurodegeneration in said subject. In some embodiments, the method reduces or prevents loss of hippocampus volume in said subject. In some embodiments, the method reduces or prevents loss or change of volume in non-hippocampus brain areas indicative of neurodegeneration such as cortical regions such as cingulate cortex or other regions in said subject. In some embodiments, the method reduces or prevents loss of one or more of hippocampus volume, basal forebrain volume, lingual gyrus volume, parahippocampal volume, and orbitofrontal cortex volume, parietal cortex or cingulate cortex volume in said subject. In some embodiments volume of lateral ventricles in measured as an indicator of general brain volume; increased size of lateral ventricular volume is an indicator of diffuse loss of brain parenchymal volume. In some embodiments, the method reduces or prevents the increase in volume of the lateral ventricles. In some embodiments, the method reduces or prevents loss of volume as detected by voxel-based MRI measurements encompassing whole brain or selected regions of brain. In some embodiments, the method reduces or prevents an increase in one or more species of amyloid beta (Aβ) level or a given amyloid species such as Ab 42, or Ab 40 or certain ratios of species such as Ab 42/40 (e.g., in a bodily fluid, such as a cerebrospinal spinal fluid (CSF) or blood or plasma). In some embodiments, the method promotes increased volume of dentate gyrus which is a part of the hippocampus. In some embodiments, the method promotes neurogenesis. In some embodiments, the method reduces or prevents an increase in other cerebrospinal and/or blood or plasma Alzheimer's disease-related biomarkers of neural degeneration such as tau or modified versions of tau, neurofilaments light chain or, SNAP-25, synaptotagmin-1, and neurogranin. In some embodiments, the method reduces or prevents an increase in other cerebrospinal and/or blood or plasma biomarkers of neuroinflammation or gliosis such as sTREM or YKL-40. In some embodiments, wherein the method reduces or prevents an increase in tau level or its level of phosphorylation or other forms of post-translational modification (e.g., in a bodily fluid, such as a cerebrospinal spinal fluid or blood or plasma). In some embodiments, the method leads to a reversal or improvement of one or more imaging or fluid-based biomarkers such as an increase in hippocampal volume or a decrease in CSF neurogranin levels. In some embodiments, said subject is younger than about 72 years of age. In some embodiments, said subject is of about 72 years of age or older. In some aspects, the disclosure provides for a method for modulating one or more neurodegeneration biomarkers or indicators in a subject in need thereof, the method comprising: administering to said subject an effective amount of a p75^{NTR} receptor modulator or a pharmaceutically acceptable salt thereof (e.g., for a time period (e.g., of at least six months)) to provide one or more of the following in said subject: (i) an amyloid beta (Aβ) level in a bodily fluid (e.g., a cerebrospinal fluid) that is lower than a corresponding reference (e.g., a corresponding pretreatment level in said bodily fluid); (ii) a tau (τ) level in a bodily fluid (e.g., a cerebrospinal fluid) increases at a rate that is lower (e.g., by at least about 1, 2, 3, 4, or 5 annual percent change (APC)) than the corresponding increase that would occur in an untreated control subject and/or is lower that a pre-treatment baseline level (e.g., a corresponding pretreatment level in said bodily fluid); (iii) a presynaptic biomarker level in a bodily fluid (e.g., a cerebrospinal fluid) that is lower (e.g., by at least about 1, 2, 3, 4, or 5 annual percent change (APC)) than a corresponding reference (e.g., a corresponding pretreatment level in said bodily fluid) or that increases at a rate (APC) that is at least about 1, 2, 3, 4, or 5 annual percent change less than the rate expected in an untreated control subject; (iv) a postsynaptic biomarker level in a bodily fluid (e.g., a cerebrospinal fluid) that is lower than a corresponding reference (e.g., a corresponding pretreatment level in said bodily fluid) or that increases at a rate (APC) that is at least about 1, 2, 3, 4, or 5 annual percent change less than the rate expected in an untreated control subject; (v) a glial marker level in a bodily fluid (e.g., a cerebrospinal fluid) that is lower (e.g., by at least about 1, 2, 3, 4, or 5 annual percent change (APC)) than a corresponding reference (e.g., a corresponding pretreatment level in said bodily fluid) or that increases at a rate (APC) that is at least about 1, 2, 3, 4, or 5 annual percent change less than the rate expected in an untreated control subject; (vi) a rate of volume change of an anatomical brain region (e.g., hippocampus) or a voxel based brain region that is lower than a corresponding rate (e.g., a corresponding pretreatment rate of volume change of said brain region) or that occurs as expected in an untreated control subject as determined by magnetic resonance imaging (MRI) imaging; (vii) a brain glucose metabolism (¹⁸F-FDG-PET) rate of decrease, in either an anatomic based region or a voxel based region, that is lower than a corresponding reference (e.g., a corresponding pretreatment rate) or rate of decline occurring in an untreated control subject; and (viii) a change in Alzheimer's Disease Assessment Scale-Cognitive (ADAS-Cog) score within a time period (e.g., of about six months) that is less than a corresponding change (e.g., a corresponding pretreatment change within a corresponding time period in said subject) or a rate occurring in an untreated control subject. In some embodiments, said Aβ is Aβ40 or Aβ42. In some embodiments, said tau is phosphorylated tau. In some embodiments, said presynaptic biomarker is selected from SNAP25 and SYT1. In some embodiments, said postsynaptic biomarker is NG36. In some embodiments, said glial or inflammatory marker is selected from sTREM2 and YKL40. In some embodiments, said ADAS is ADAS11 or ADAS13. In some embodiments, said subject is younger than about 72 years of age. In some embodiments, said subject is of about 72 years of age or older.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a diagram showing the administration schedule of the phase IIa trial for LM11A-31-BHS—PK (the salt of Formula (II)) in patients with mild to moderate Alzheimer's disease.

FIG. 2 illustrates a randomization table.

FIG. 3 illustrates a randomization process.

FIG. 4 illustrates plasma aminoethyl morpholine metabolite (AEM)-pharmacokinetic (PK) data from 200 mg safety population from visit 2, 3, 4 and 5. Values below the limit of quantification (BLQ) were treated as BLQ/2.

FIG. 5 depicts plasma AEM-PK data from 400 mg safety population from visit 2, 3, 4 and 5. Values below the limit of quantification (BLQ) were treated as BLQ/2.

FIG. 6 depicts plasma LM11A-31-BHS—PK data from 200 mg safety population from visit 2, 3, 4 and 5. Values below the limit of quantification (BLQ) were treated as BLQ/2.

FIG. 7 depicts plasma LM11A-31-BHS—PK data from 400 mg safety population from visit 2, 3, 4 and 5. Values below the limit of quantification (BLQ) were treated as BLQ/2.

FIG. 8 depicts CSF LM11A-31-BHS—PK data from 200 mg and 400 mg safety population from visit 5.

FIG. 9 depicts plasma AEM levels (Y-axis) compared to CSF LM11A-31-BHS levels (X-axis).

FIG. 10 depicts Plasma LM11A-31-BHS levels (Y-axis) compared to CSF LM11A-31-BHS levels (X-axis).

FIG. 11 depicts a flow diagram showing disposition of patients in the phase IIa trial.

FIG. 12 depicts a bar chart showing reasons for withdrawal within the subject groups within the phase IIa trial.

FIG. 13 depicts a bar chart showing the distribution of sex within the three treatment groups of the phase IIa trial.

FIG. 14 depicts a bar chart showing the distribution of age within the three treatment groups of the phase IIa trial.

FIG. 15 depicts a bar chart showing the distribution of the HIS between the dosage groups of the safety population.

FIG. 16 depicts a bar chart showing ApoE distribution within the different groups of the safety population.

FIG. 17 depicts ApoE distribution within the different groups of the safety population.

FIG. 18 depicts ApoE distribution within the different groups of the intention to treat population.

FIG. 19 depicts ApoE distribution within the different groups of the per protocol population.

FIGS. 20A-20G depict the percentage of blood pressure interpretations for the diastolic blood pressure (DBP), systolic blood pressure (SBP) of the different dosage groups and overall for different timepoints, the early discontinuation visit (EDV) and the last observation carried forward (LOCF) for the different dosage groups in the phase IIa trial. The groups include screening (FIG. 20A), Baseline (FIG. 20B), week 4 (FIG. 20C), week 12 (FIG. 20D), week 26 (FIG. 20E), EDV (FIG. 20F), and LOCF (FIG. 20G).

FIGS. 21A-21F depict the percentage of ECG interpretations for the different dosage groups and overall for different timepoints, the early discontinuation visit (EDV) and the last observation carried forward (LOCF) in the phase IIa trial. The groups include screening (FIG. 21A), week 4 (FIG. 21B), week 12 (FIG. 21C), week 26 (FIG. 21D), EDV (FIG. 21E), and LOCF (FIG. 21F).

FIG. 22 depicts a line graph of the eosinophils values of the safety population receiving any dosage of LM11A-31-BHS in the phase IIa trial.

FIG. 23 depicts a line graph of the eosinophils values of the safety population receiving placebo in the phase IIa trial.

FIG. 24 depicts a clustered bar graph of eosinophil Means ([/μl]) for visits per placebo and verum groups in the phase IIa trial.

FIG. 25 depicts mechanism engagement and biomarkers.

FIG. 26 depicts preclinical studies in mice point to degenerative mechanisms and human biomarkers directly relevant to those respective degenerative mechanisms.

FIG. 27 depicts estimating human brain exposure to drug.

FIG. 28 depicts phase 2a RCT safety and exploratory endpoint trial.

FIG. 29A depicts an exemplary Safety/PK defined population (LM11A31 200 mg bid 78, LM11A31 400 mg bid 83, Placebo control 81 patients). FIGS. 29B-29E illustrate observations in regard to age, MMSE, ratio of Aβ42 to Aβ40 (AJ342/40), and ratio of phosphorylated tau to Aβ42 (p-tau/AD42), respectively.

FIG. 30 depicts safety results. 17 subjects left the trial because of adverse events (AEs).

FIGS. 31A-31D depict the progression as indicated by biomarkers and clinical testing of the placebo group in the population of mild-moderate AD over the 26-week study period. Four outcome domains include: CSF biomarkers (FIG. 31A), sMRI (FIG. 31B), cognition (FIG. 31C), and FDG-PET (FIG. 31D).

FIGS. 32A-32C depict exploratory outcomes for domain 1-CSF AD core biomarkers related with Ab 42 (FIG. 32A) or Aβ40 (FIG. 32B), and related to modulation the p75 receptor (FIG. 32C) by the drug and the effect of this modulation on APP processing to Ab 42 and resulting CSF levels of Ab 42.

FIGS. 33A-33B depict exploratory outcomes for CFS AD core biomarkers related with Ab. FIG. 33A illustrates placebo vs. combined drug; and FIG. 33B illustrates placebo vs. different doses (200 mg and 400 mg).

FIGS. 34A-34B depict exploratory outcomes for domain 1-CSF AD core biomarkers related with CSF tau levels: tau (FIG. 34A) and p-tau (FIG. 34B).

FIGS. 35A-35B depict exploratory outcomes for CFS AD core biomarkers related with tau and p-tau. FIG. 35A illustrates placebo vs. combined drug; and FIG. 35B illustrates placebo vs. different doses (200 mg and 400 mg).

FIGS. 36A-36B depict exploratory outcomes for domain 1-CSF pre-synaptic biomarkers related with levels of SYT1 (FIG. 36A) and SNAP-25 (FIG. 36B).

FIGS. 37A-37B depict exploratory outcomes for CFS pre-synaptic biomarkers related with SYT1 and SNAP-25. FIG. 37A illustrates placebo vs. combined drug; and FIG. 37B illustrates placebo vs. different doses (200 mg and 400 mg).

FIG. 38 depicts exploratory outcomes for domain 1-CSF post-synaptic biomarkers related with neurogranin.

FIGS. 39A-39B depict exploratory outcomes for CFS AD post-synaptic biomarkers related with NG36. FIG. 39A illustrates placebo vs. combined drug; and FIG. 39B illustrates placebo vs. different doses (200 mg and 400 mg).

FIGS. 40A-40B depict exploratory outcomes for domain 1-CSF glial/neuroinflammatory biomarkers related with sTREM2 (FIG. 40A) and YKL-40 (FIG. 40B).

FIGS. 41A-41B depict exploratory outcomes for CSF glial biomarkers related with sTREM2 and YKL-40. FIG. 41A illustrates placebo vs. combined drug; and FIG. 41B illustrates placebo vs. different doses (200 mg and 400 mg).

FIG. 42 depicts exploratory outcomes for domain 2 using Hippocampal sMRI (structural MRI).

FIG. 43 depicts preprocessing strategy for analysis of structural MRI using voxel-based morphometry. Time 1 indicates pretreatment MRI imaging and time 2 indicates post treatment MRI imaging.

FIG. 44 depicts T1-weighted structural MRI using whole brain voxel-wise analysis. It is demonstration of flexible factorial generalized linear model (GLM) analysis matrix for brain voxel-wise structural analysis.

FIG. 45 depicts whole brain results of T1-weighted structural MRI analysis.

FIG. 46A-46C depict T1-weighted structural MRI using Group x Time interaction patterns. FIG. 46A shows the MRI image. FIG. 46B illustrates placebo vs. combined drug; and FIG. 46C illustrates placebo vs. different doses (200 mg and 400 mg).

FIG. 47 depicts T1-weighted structural MRI using the MCS (Monte Carlo Simulation) analysis and dose effects.

FIGS. 48A-48C depict cortical thickness changes in anterior cingulate region area 33 prime. FIG. 48B illustrates low dose vs. placebo; and FIG. 48C illustrates high dose vs. placebo.

FIGS. 49A-49B depict exploratory outcomes for domain 3 related with cognition: ADAS-13 (FIG. 49A) and ADAS-11 (FIG. 49B).

FIGS. 50A-50B depict exploratory outcomes for cognition AD core markers related with ADAS. FIG. 50A illustrates placebo vs. combined drug; and FIG. 50B illustrates placebo vs. different doses (200 mg and 400 mg).

FIG. 51A depicts exploratory outcomes for domain 4 related with FDG-PET as a marker of synaptic function. FIG. 51B illustrates MCIMaskSROI APC for the placebo and treatment groups.

FIG. 52 depicts exploratory outcomes for domain 4 FDG-PET with post-hoc MCS.

FIG. 53 depicts Co-registration to MRI and SUVR using FDG PET.

FIG. 54 depicts FDG PET using whole brain voxel-wise analysis.

FIG. 55 depicts whole brain results of FDG PET.

FIGS. 56A-56C depicts FDG PET using Group x Time interaction patterns. FIG. 56A shows the image. FIG. 56B illustrates placebo vs. combined drug; and FIG. 56C illustrates placebo vs. different doses (200 mg and 400 mg).

FIG. 57 depicts FDG PET using MCS analysis.

FIG. 58 depicts summary of the biomarkers showing a statistically significant effect of drug on multiple mechanisms and the ability of the drug to engage with and affect targeted disease mechanisms.

FIGS. 59A-59D depict summary of the placebo progression measured by the measures in the 4 outcome domains and the effects and trends of the drug on slowing disease progression. Four outcome domains include: CSF biomarkers (FIG. 59A), sMRI (FIG. 59B), cognition (FIG. 59C), and FDG-PET (FIG. 59D).

FIG. 60 depicts summary of dose selection of efficacy trial.

FIG. 61 depicts FDG PET and sMRI results.

FIGS. 62A-62C depict age effect analysis for CSF total tau biomarker(s) in regard with full sample (FIG. 62A), younger population<72 years (FIG. 62B), and older population≥72 years (FIG. 62C).

FIGS. 63A-63C depict age effect analysis for CSF SNAP-25 presynaptic biomarker in regard with full sample (FIG. 63A), younger population<72 years (FIG. 63B), and older population>72 years (FIG. 63C).

FIGS. 64A-64C depict age effect analysis for YKL-40 biomarker in regard with full sample (FIG. 64A), younger population<72 years (FIG. 64B), and older population>72 years (FIG. 64C).

FIGS. 65A-65C depict summary of all CSF biomarkers examined in phase 2a study in regard with full sample (FIG. 65A), younger population<72 years (FIG. 65B), and older population≥72 years (FIG. 65C). The CSF biomarkers included Ab 40, Ab 42, tau, phosphotau, SNAP 25, NG36, SYT1, NFL21, AchE, YKL_40, and sTREM2.

FIGS. 66A-66C depict T1-weighted structural MRI analysis. FIG. 66A shows the MRI image. FIG. 66B illustrates placebo vs. treatment; and FIG. 66C illustrate age group effects.

### DETAILED DESCRIPTION OF THE INVENTION

The disclosure provides compounds, salts and methods of use thereof for the prevention and treatment of Alzheimer's disease.

### Definitions

It is to be understood that the terminology used herein is for the purpose of describing particular embodiments only and is not intended to be limiting.

Unless defined otherwise, all technical and scientific terms used herein have the same meaning as commonly understood to one of ordinary skill in the art to which the present application belongs. Although any methods and materials similar or equivalent to those described herein can be used in the practice or testing of the present application, representative methods and materials are herein described.

Following long-standing patent law convention, the terms “a,” “an,” and “the” refer to “one or more” when used in this application, including the claims. Thus, for example, reference to “a carrier”” includes mixtures of one or more carriers, two or more carriers, and the like.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the present specification and attached claims are approximations that can vary depending upon the desired properties sought to be obtained by the present application. Generally the term “about,” as used herein when referring to a measurable value such as an amount of weight, time, dose, etc. is meant to encompass in one example variations of ±20% or ±10%, in another example ±5%, in another example ±1%, and in yet another example ±0.10% from the specified amount, as such variations are appropriate to perform the disclosed method.

The term “alkyl,” alone or in combination, refers to an optionally substituted straight-chain or branched-chain alkyl radical having from 1 to 20 carbon atoms. The term also includes optionally substituted straight-chain or branched-chain alkyl radicals having from 1 to 6 carbon atoms as well as those having from 1 to 4 carbon atoms. Examples of alkyl radicals include methyl, ethyl, n-propyl, isopropyl, n-butyl, isobutyl, sec-butyl, tert-butyl, tert-amyl, pentyl, hexyl, heptyl, octyl and the like. “Branched” refers to an alkyl group in which a lower alkyl group, such as methyl, ethyl or propyl, is attached to a linear alkyl chain. “Lower alkyl” refers to an alkyl group having 1 to 8 carbon atoms (i.e., a C₁₋₈ alkyl), e.g., 1, 2, 3, 4, 5, 6, 7, or 8 carbon atoms. “Higher alkyl” refers to an alkyl group having about 10 to 20 carbon atoms, e.g., 10, 11, 12, 13, 14, 15, 16, 17, 18, 19, or 20 carbon atoms. In certain embodiments, “alkyl” refers, in particular, to C₁₋₈ straight-chain alkyls. In other embodiments, “alkyl” refers, in particular, to C₁₋₈ branched-chain alkyls. Alkyl groups can be optionally substituted.

The term “alkenyl,” alone or in combination, refers to an optionally substituted straight-chain or branched-chain hydrocarbon radical having one or more carbon-carbon double-bonds and having from 2 to 18 carbon atoms. Alkenyl includes optionally substituted straight-chain or branched-chain hydrocarbon radicals having one or more carbon-carbon double bonds and having from 2 to 6 carbon atoms such as from 2 to 4 carbon atoms. Examples of alkenyl radicals include ethenyl, propenyl, butenyl, 1,4-butadienyl and the like. Suitable alkenyl groups include allyl. The terms “allylic group” or “allyl” refer to the group —CH₂HC═CH₂ and derivatives thereof formed by substitution. Thus, the terms alkenyl and/or substituted alkenyl include allyl groups, such as but not limited to, allyl, methylallyl, di-methylallyl, and the like. The term “allylic position” or “allylic site” refers to the saturated carbon atom of an allylic group. Thus, a group, such as a hydroxyl group or other substituent group, attached at an allylic site can be referred to as “allylic.” “1-alkenyl” refers to alkenyl groups where the double bond is between the first and second carbon atom.

The term “alkynyl,” alone or in combination, refers to an optionally substituted straight-chain or branched-chain hydrocarbon radical having one or more carbon-carbon triple-bonds and having from 2 to 12 carbon atoms. Alkynyl includes optionally substituted straight-chain or branched-chain hydrocarbon radicals having one or more carbon-carbon triple bonds and having from 2 to 6 carbon atoms such as from 2 to 4 carbon atoms. Examples of alkynyl radicals include ethynyl, propynyl, butynyl and the like. “1-alkynyl” refers to alkynyl groups where the triple bond is between the first and second carbon atom.

“Cycloalkyl” refer to a non-aromatic mono- or multicyclic ring system of 3 to 10 carbon atoms, e.g., 3, 4, 5, 6, 7, 8, 9, or 10 carbon atoms, such as from 3 to 6 carbon atoms. The cycloalkyl group can be optionally partially unsaturated. The cycloalkyl group also can be optionally substituted as defined herein. Representative monocyclic cycloalkyl rings include, but are not limited to, cyclopropyl, cyclobutyl, cyclopentyl, cyclohexyl, cycloheptyl, and the like. Further, the cycloalkyl group can be optionally substituted with a linking group, such as an alkylene group as defined hereinabove, for example, methylene, ethylene, propylene, and the like. In such cases, the cycloalkyl group can be referred to as, for example, cyclopropylmethyl, cyclobutylmethyl, and the like. Additionally, multicyclic cycloalkyl rings include adamantyl, octahydronaphthyl, decalin, camphor, camphane, and noradamantyl.

The term “heterocycloalkyl” refers to a cyclic group of 3 to 6 atoms, or 3 to 10 atoms, containing at least one heteroatom. In one aspect, these groups contain 1 to 3 heteroatoms. Suitable heteroatoms include, for example, oxygen, sulfur, and nitrogen. Heterocyclic groups may be attached through any substitutable atom, such as a nitrogen or through a carbon atom in the ring. Suitable heterocyclic groups include pyrrolidinyl, morpholino, morpholinoethyl, and pyridyl. Such groups may be substituted.

The term “aryl” refers to aromatic groups which have 5-14 ring atoms and at least one ring having a conjugated pi electron system and includes carbocyclic aryl, heteroaryl and biaryl groups, all of which may be optionally substituted. The term “aryl” is used herein to refer to an aromatic substituent that can be a single aromatic ring, or multiple aromatic rings that are fused together, linked covalently, or linked to a common group, such as, but not limited to, a methylene or ethylene moiety. The common linking group also can be a carbonyl, as in benzophenone, or oxygen, as in diphenylether, or nitrogen, as in diphenylamine. In particular embodiments, the term “aryl” includes cyclic aromatic comprising about 5 to about 10 carbon atoms, e.g., 5, 6, 7, 8, 9, or 10 carbon atoms, and including 5- and 6-membered hydrocarbon and heterocyclic aromatic rings. Examples of aryl groups include, but are not limited to, cyclopentadienyl, phenyl, furan, thiophene, pyrrole, pyran, pyridine, imidazole, benzimidazole, isothiazole, isoxazole, pyrazole, pyrazine, triazine, pyrimidine, quinoline, isoquinoline, indole, carbazole, and the like, all optionally substituted. Thus, as used herein, the term “substituted aryl” includes aryl groups, as defined herein, in which one or more atoms or functional groups of the aryl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto. Unless otherwise specified, substituents on an aryl group may be independently selected at each occurrence from alkyl, aryl, aralkyl, hydroxyl, alkoxyl, haloalkyl, aryloxyl, aralkyloxyl, carboxyl, acyl, halo, nitro, alkoxycarbonyl, aryloxycarbonyl, aralkoxycarbonyl, acyloxyl, acylamino, carbamoyl, alkylcarbamoyl, dialkylcarbamoyl, arylthio, alkylthio, and —NR′R″, wherein R′ and R″ can each be independently hydrogen, alkyl, substituted alkyl, aryl, substituted aryl, and aralkyl.

A structure represented generally by a formula such as:

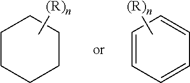

as used herein refers to a 6-carbon ring structure comprising a substituent R group, wherein the R group can be present or absent, and when present, one or more R groups can each be substituted on one or more available carbon atoms of the ring structure. The presence or absence of the R group and number of R groups is determined by the value of the integer n. Each R group, if more than one, is substituted on an available carbon of the ring structure rather than on another R group. For example, the structure:

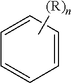

wherein n is an integer from 0 to 2 comprises compound groups including, but not limited to:

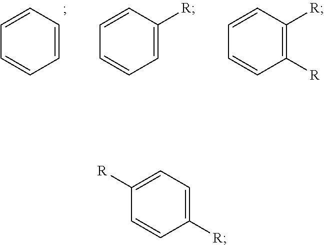

and the like.

The structure:

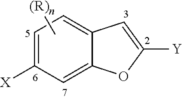

wherein n is one (1) comprises compound groups including:

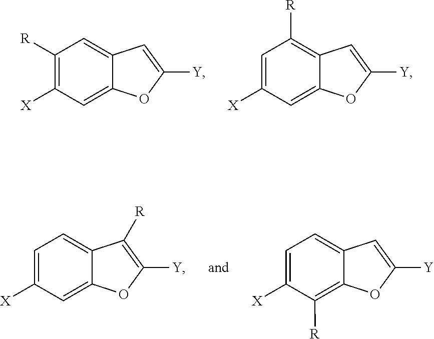

wherein the one (1) R substituent can be attached at any carbon on the benzofuran parent structure not occupied by another designated substituent, as in this case carbon 6 is substituted by X and carbon 2 is substituted by Y.

A dashed line representing a bond in a cyclic ring structure indicates that the bond can be either present or absent in the ring.

“Carbocyclic aryl” groups are groups wherein the ring atoms on the aromatic ring are carbon atoms. Carbocyclic aryl groups include monocyclic carbocyclic aryl groups and polycyclic or fused compounds such as optionally substituted naphthyl groups.

“Heteroaryl” groups are groups having from 1 to 4 heteroatoms as ring atoms in the aromatic ring and the remainder of the ring atoms being carbon atoms. Suitable heteroatoms include oxygen, sulfur, nitrogen, and selenium. Suitable heteroaryl groups include furanyl, thienyl, pyridyl, pyrrolyl, N-lower alkyl pyrrolyl, pyridyl-N-oxide, pyrimidyl, pyrazinyl, imidazolyl, and the like, all optionally substituted.

The phrase “carbocyclic ring” refers to a saturated or unsaturated monocyclic or bicyclic ring in which all atoms of all rings are carbon. Thus, the term includes cycloalkyl and carbocyclic aryl rings.

The phrase “heterocyclic ring” refers to a saturated or unsaturated monocyclic or bicyclic ring having from 1 to 4 heteroatoms as ring atoms in the aromatic ring and the remainder of the ring atoms being carbon atoms. Thus, the term includes heterocycloalkyl and heteroaryl rings.

In certain embodiments, “optionally substituted” or “substituted” includes groups substituted by one or more substituents independently selected from halogen, —NO₂, —CN, —OR¹⁰⁰, —SR¹⁰⁰, —N(R¹⁰⁰)₂, —S(═O)R¹⁰⁰, —S(═O)₂R¹⁰⁰, —S(═O)₂N(R¹⁰⁰)₂, —NR¹⁰⁰S(═O)₂R¹⁰⁰, —C(O)R¹⁰⁰, —C(O)OR¹⁰⁰, —OC(O)R¹⁰⁰, —OC(O)OR¹⁰⁰, —OC(O)N(R¹⁰⁰)₂, —NR¹⁰⁰C(O)R¹⁰⁰, —C(O)N(R¹⁰⁰)₂, ═O, ═S, ═N(R¹⁰⁰), —P(O)(OR¹⁰⁰)₂, —OP(O)(OR¹⁰⁰)₂; C₁₋₁₀ alkyl, C₂₋₁₀ alkenyl, and C₂₋₁₀ alkynyl, each of which is independently optionally substituted at each occurrence with one or more substituents selected from halogen, —NO₂, —CN, —OR¹⁰⁰, —SR¹⁰⁰, —N(R¹⁰⁰)₂, —S(═O)R¹⁰⁰, —S(═O)₂R¹⁰⁰, —S(═O)₂N(R¹⁰⁰)₂, —NR¹⁰⁰S(═O)₂R¹⁰⁰, —C(O)R¹⁰⁰, —C(O)OR¹⁰⁰, —OC(O)R¹⁰⁰, —OC(O)OR¹⁰⁰, —OC(O)N(R¹⁰⁰)₂, —NR¹⁰⁰C(O)R¹⁰⁰, —C(O)N(R¹⁰⁰)₂, ═O, ═S, ═N(R¹⁰⁰), —P(O)(OR¹⁰⁰)₂, —OP(O)(OR¹⁰⁰)₂, C₃₋₁₂ carbocycle and 3- to 12-membered heterocycle; and C₃₋₁₂ carbocycle and 3- to 12-membered heterocycle, wherein each C₃₋₁₂ carbocycle and 3- to 12-membered heterocycle is independently optionally substituted with one or more substituents selected from halogen, —NO₂, —CN, —OR¹⁰⁰, —SR¹⁰⁰, —N(R¹⁰⁰)₂, —S(═O)R¹⁰⁰, —S(═O)₂R¹⁰⁰, —S(═O)₂N(R¹⁰⁰)₂, —NR¹⁰⁰S(═O)₂R¹⁰⁰, —C(O)R¹⁰⁰, —C(O)OR¹⁰⁰, —OC(O)R¹⁰⁰, —OC(O)OR¹⁰⁰, —OC(O)N(R¹⁰⁰)₂, —NR¹⁰C(O)R¹⁰⁰, —C(O)N(R¹⁰⁰)₂, ═O, ═S, ═N(R¹⁰⁰), —P(O)(OR¹⁰⁰)₂, —OP(O)(OR¹⁰⁰)₂, C₁₋₆ alkyl, C₁₋₆ haloalkyl, C₂₋₆ alkenyl, and C₂₋₆ alkynyl, wherein R¹⁰⁰ at each occurrence is independently selected from hydrogen; and C₁₋₂₀ alkyl, C₂₋₂₀ alkenyl, C₂₋₂₀ alkynyl, C₃₋₁₂ carbocycle and 3- to 12-membered heterocycle, each of which may be optionally substituted by halogen, —CN, —NO₂, —OH and —OCH₃. In certain embodiments, the term “optionally substituted” or “substituted” includes groups substituted by one to four substituents, independently selected from lower alkyl, lower aryl, lower aralkyl, lower alicyclic, heterocycloalkyl, hydroxyl, lower alkoxy, lower aryloxy, perhaloalkoxy, aralkoxy, heteroaryl, heteroaryloxy, heteroarylalkyl, heteroaralkoxy, azido, amino, guanidino, amidino, halo, lower alkylthio, oxo, acylalkyl, carboxy esters, carboxyl,-carboxamido, nitro, acyloxy, aminoalkyl, alkylaminoaryl, alkylaryl, alkylaminoalkyl, alkoxyaryl, arylamino, aralkylamino, phosphono, sulfonyl, -carboxamidoalkylaryl, -carboxamidoaryl, hydroxyalkyl, haloalkyl, alkylaminoalkylcarboxy-, aminocarboxamidoalkyl-, cyano, lower alkoxyl, lower perhaloalkyl, and arylalkyloxyalkyl.

“Tautomers” are structurally distinct isomers that interconvert by tautomerization. “Tautomerization” is a form of isomerization and includes prototropic or proton-shift tautomerization, which is considered a subset of acid-base chemistry. “Prototropic tautomerization” or “proton-shift tautomerization” involves the migration of a proton accompanied by changes in bond order, often the interchange of a single bond with an adjacent double bond. Where tautomerization is possible (e.g., in solution), a chemical equilibrium of tautomers can be reached. An example of tautomerization is keto-enol tautomerization. A specific example of keto-enol tautomerization is the interconversion of pentane-2,4-dione and 4-hydroxypent-3-en-2-one tautomers. Another example of tautomerization is phenol-keto tautomerization. A specific example of phenol-keto tautomerization is the interconversion of pyridin-4-ol and pyridin-4(1H)-one tautomers.

In some embodiments, the compounds described by the presently disclosed subject matter contain a linking group. As used herein, the term “linking group” comprises a chemical moiety which is bonded to two or more other chemical moieties to form a stable structure. In certain embodiments, the linking group, e.g., methylene, ethylene, links a moiety, e.g., an aryl or heteroaryl group, to the remainder of the structure.

“Alkylene” refers to a straight or branched bivalent aliphatic hydrocarbon group having from 1 to 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched or cyclic. The alkylene group can be optionally substituted with one or more substituents. Exemplary alkylene groups include methylene (—CH₂—); ethylene (—CH₂—CH₂—); propylene (—(CH₂)₃—); cyclohexylene (—C₆H₁₀—); —CH═CH—CH═CH—; —CH═CH—CH₂—; —(CH₂)_{q}—N(R)—(CH₂)ᵣ—, wherein each of q and r is independently an integer from 0 to 20, e.g., 0, 1, 2, 3, 4, 5, 6, 7, 8, 9, 10, 11, 12, 13, 14, 15, 16, 17, 18, 19, or 20, and R is hydrogen or lower alkyl. In certain embodiments, an alkylene group has from 1 to 6 carbon atoms such as from 1 to 3 carbon atoms.

The term “alkenylene” denotes a straight or branched bivalent aliphatic hydrocarbon group having from 2 to 20 carbon atoms with at least one carbon-carbon double bond. The alkenylene group can be optionally substituted with one or more substituents. Representative alkenylene groups include, but are not limited to, ethenylene, propenylene, 1- or 2-butenylene, 1-, or 2-pentylene, and the like.

As used herein, the term “acyl” refers to an group represented by R—C(═O)—, wherein R is, for example, an alkyl or an aryl group as defined herein. Specific examples of acyl groups include acetyl and benzoyl.

“Alkoxyl” refers to an alkyl-O— group wherein alkyl is as previously described. The term “alkoxyl” as used herein can refer to C₁₋₂₀ inclusive, linear, branched, or cyclic, saturated or unsaturated oxo-hydrocarbon chains, including, for example, methoxyl, ethoxyl, propoxyl, isopropoxyl, butoxyl, t-butoxyl, and pentoxyl.

“Aryloxyl” refers to an aryl-O— group wherein the aryl group is as previously described, including a substituted aryl. The term “aryloxyl” as used herein can refer to optionally substituted phenyloxyl.

“Aralkyl” refers to an aryl-alkyl- group wherein aryl and alkyl are as previously described, and included substituted aryl and substituted alkyl. Exemplary aralkyl groups include benzyl, phenylethyl, and naphthylmethyl.

“Aralkyloxyl” refers to an aralkyl-O— group wherein the aralkyl group is as previously described. An exemplary aralkyloxyl group is benzyloxyl.

“Dialkylamino” refers to an —NRR′ group wherein each of R and R′ is independently selected from optionally substituted alkyl groups as previously described. Exemplary alkylamino groups include ethylmethylamino, dimethylamino, and diethylamino.

“Alkoxycarbonyl” refers to an alkoxyl-C(O)— group. Exemplary alkoxycarbonyl groups include methoxycarbonyl, ethoxycarbonyl, butyloxycarbonyl, and t-butyloxycarbonyl.

“Aryloxycarbonyl” refers to an aryloxyl-CO— group. Exemplary aryloxycarbonyl groups include phenoxycarbonyl and naphthoxycarbonyl.

“Aralkoxycarbonyl” refers to an aralkyl-O—CO— group. An exemplary aralkoxycarbonyl group is benzyloxycarbonyl.

“Carbamoyl” refers to an H₂N—CO— group.

“Alkylcarbamoyl” refers to a R′RN—CO— group wherein one of R and R′ is hydrogen and the other of R and R′ is optionally substituted alkyl as previously described.

“Dialkylcarbamoyl” refers to a R′RN—CO— group wherein each of R and R′ is independently optionally substituted alkyl as previously described.

“Acyloxyl” refers to an acyl-O— group wherein acyl is as previously described.

“Acylamino” refers to an acyl-NH— group wherein acyl is as previously described.

The term “amino” refers to the —NH₂ group and amino may be optionally substituted.

The term “carbonyl” refers to the —C(O)— group.

The term “carboxyl” refers to the —COOH group.

The term “cyano” refers to the —CN group.

The terms “halo,” “halide,” or “halogen” as used herein refer to fluoro, chloro, bromo, and iodo groups.

The term “hydroxyl” refers to the —OH group.

The term “hydroxyalkyl” refers to an alkyl group substituted with one or more —OH substituents.

The term “haloalkyl” refers to an alkyl group with one or more independently selected halogen substituents.

The term “mercapto” refers to the —SH group.

The term “oxo” refers to =O.

The term “nitro” refers to the —NO₂ group.

The term “thio” refers to a compound described previously herein wherein a carbon or oxygen atom is replaced by a sulfur atom.

The term “sulfate” refers to the —SO₄ group.

The term “treatment” as used herein covers any treatment of a disease and/or condition in an animal or mammal, particularly a human, and includes: (i) preventing a disease, disorder and/or condition and/or symptoms from occurring in a person which can be predisposed to the disease, disorder and/or condition, or at risk for being exposed to an agent that can cause the disease, disorder, and/or condition and/or symptoms; but, has not yet been diagnosed as having it; (ii) inhibiting the disease, disorder and/or condition, and/or symptoms i.e., arresting its development; (iii) slowing progress of the disease, disorder and/or condition, and/or symptoms; and (iv) relieving the disease, disorder and/or condition, and/or symptoms i.e., causing regression of the disease, disorder and/or condition; (v) augmenting a mechanism, such as modulating p75NTR signaling, that can lead to reduced symptoms and improved function.

The terms “modulate” and “modulation” as used herein is used in the common manner of the field as to regulate or adjust to a certain degree.

The term “derivative” as used herein refers to a compound chemically modified so as to differentiate it from a parent compound. Such chemical modifications can include, for example, replacement of hydrogen by an alkyl, acyl, or amino group. A derivative compound can be modified by, for example, glycosylation, pegylation, or any similar process that retains at least one biological or immunological function of the compound from which it was derived.

The term “hydrophilicity” is used in the common manner of the field as having an affinity for water; readily absorbing and/or dissolving in water.

The term “lipophilicity” is used in the common manner of the field as having an affinity for, tending to combine with, or capable of dissolving in lipids.

The term “amphipathicity,” as used herein, describes a structure having discrete hydrophobic and hydrophilic regions. Thus, one portion of the structure interacts favorably with aqueous and other polar media, while another portion of the structure interacts favorably with non-polar media.

The term “solubility” as used herein, describes the maximum amount of solute that will dissolve in a given amount of solvent at a specified temperature.

The term “bioavailability” as used herein refers to the systemic availability, blood/plasma levels, of a given amount of compound administered to a subject. The term further encompasses the rate and extent of absorption of compound that reaches the site of action.

As used herein, “solvate” means a complex formed by the combination of solvent molecules with molecules or ions of the compound or salt of the disclosure. Examples of hydrate include, but are not limited to, hemihydrate, monohydrate, dihydrate, trihydrate, hexahydrate, etc. Solvates, including hydrates, may be found in stoichiometric ratios, for example, with two, three, four salt molecules of the disclosure per solvate or per hydrate molecule. Solvents used for crystallization, such as alcohols, especially methanol and ethanol; aldehydes; ketones, especially acetone; esters, e.g. ethyl acetate; may be embedded in the crystal grating.

The term “prodrug” as used herein refers to any compound that when administered to a biological system generates the drug substance (a biologically active compound) in steps involving, for example, spontaneous chemical reaction(s), enzyme catalyzed chemical reaction(s), or both.

Where the compounds of the present invention have at least one asymmetric center, they may accordingly exist as enantiomers. Where the compounds possess two or more asymmetric centers, they may additionally exist as diastereoisomers. It is to be understood that all such stereoisomers and mixtures thereof in any proportion are encompassed within the scope of the present invention. Where the compounds possess geometrical isomers, all such isomers and mixtures thereof in any proportion are encompassed within the scope of the present invention. Where so indicated in the claims herein, if a single enantiomer of the potentially optically active heterocyclic compounds disclosed is desired, for either health or efficacy reasons, preferably it is present in an enantiomeric excess of at least about 80%, or at least about 90%, or at least about 95%, or at least about 98%, or at least about 99%, or at least about 99.5%.

Polymorphism can be characterized as the ability of a compound to crystallize into different crystal forms, while maintaining the same chemical formula. A crystalline polymorph of a given drug substance is chemically identical to any other crystalline polymorph of that drug substance in containing the same atoms bonded to one another in the same way, but differs in its crystal forms, which can affect one or more physical properties, such as stability, solubility, melting point, bulk density, flow properties, bioavailability, etc.

The term “composition” denotes one or more substance in a physical form, such as solid, liquid, gas, or a mixture thereof. One example of composition is a pharmaceutical composition, i.e., a composition related to, prepared for, or used in medical treatment.

The term “carboxylic acid” refers to an organic acid characterized by one or more carboxyl groups, such as acetic acid and oxalic acid. “Sulfonic acid” refers to an organic acid with the general formula of R—(S(O)₂—OH)ₙ, wherein R is an organic moiety and n is an integer above zero, such as 1, 2, and 3. The term “polyhydroxy acid” refers to a carboxylic acid containing two or more hydroxyl groups. Examples of polyhydroxy acid include, but are not limited to, lactobionic acid, gluconic acid, and galactose.

“Neurotrophin mimetic compound” denotes an organic compound that resembles the biological function or activity of neurotrophin.

As used herein, “pharmaceutically acceptable” means suitable for use in contact with the tissues of humans and animals without undue toxicity, irritation, allergic response, and the like, commensurate with a reasonable benefit/risk ratio, and effective for their intended use within the scope of sound medical judgment.

“Salts” include derivatives of an active agent, wherein the active agent is modified by making acid or base addition salts thereof. Preferably, the salts are pharmaceutically acceptable salts. Such salts include, but are not limited to, pharmaceutically acceptable acid addition salts, pharmaceutically acceptable base addition salts, pharmaceutically acceptable metal salts, ammonium and alkylated ammonium salts. Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids, sulphates, nitrates, phosphates, perchlorates, borates, acetates, benzoates, hydroxynaphthoates, glycerophosphates, ketoglutarates and the like. Base addition salts include but are not limited to, ethylenediamine, N-methyl-glucamine, lysine, arginine, omithine, choline, N,N′-dibenzylethylenediamine, chloroprocaine, diethanolamine, procaine, N-benzylphenethylamine, diethylamine, piperazine, tris-(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e.g., lysine and arginine dicyclohexylamine and the like. Examples of metal salts include lithium, sodium, potassium, magnesium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like. Examples of organic bases include lysine, arginine, guanidine, diethanolamine, choline and the like. Standard methods for the preparation of pharmaceutically acceptable salts and their formulations are well known in the art, and are disclosed in various references, including for example, Remington: The Science and Practice of Pharmacy, A. Gennaro, ed., 20th edition, Lippincott, Williams & Wilkins, Philadelphia, PA.

As used herein, “solvate” means a complex formed by solvation (the combination of solvent molecules with molecules or ions of the active agent of the present invention), or an aggregate that consists of a solute ion or molecule (the active agent of the present invention) with one or more solvent molecules. In the present invention, the preferred solvate is hydrate. Examples of hydrate include, but are not limited to, hemihydrate, monohydrate, dihydrate, trihydrate, hexahydrate, etc. It should be understood by one of ordinary skill in the art that the pharmaceutically acceptable salt of the present compound may also exist in a solvate form. The solvate is typically formed via hydration which is either part of the preparation of the present compound or through natural absorption of moisture by the anhydrous compound of the present invention. Solvates including hydrates may be consisting in stoichiometric ratios, for example, with two, three, four salt molecules per solvate or per hydrate molecule. Another possibility, for example, that two salt molecules are stoichiometric related to three, five, seven solvent or hydrate molecules. Solvents used for crystallization, such as alcohols, especially methanol and ethanol; aldehydes; ketones, especially acetone; esters, e.g. ethyl acetate; may be embedded in the crystal grating. Preferred are pharmaceutically acceptable solvents.

The term “substantially similar” as used herein means an analytical spectrum, such as XRD pattern, Raman spectroscopy, and etc., which resembles the reference spectrum to a great degree in both the peak locations and their intensity.

The terms “excipient,” “carrier,” and “vehicle” are used interexchangeably throughout this application and denote a substance with which a compound of the present invention is administered.

As described herein, the dataset indicated as “a combined drug” are derived from corresponding analyses that combine both datasets indicated as “200 mg” and “400 mg” for the purpose of statistical tests.

As described herein, unless specified otherwise, the term “baseline” or “corresponding reference,” when used in describing the effect(s) of a therapeutic agent may refer to a corresponding measurement determined prior to the treatment, a placebo control where a biologically inert substance is used, or a corresponding cohort or subject determined to have identical or meaningfully similar conditions.

The term “neurodegeneration biomarkers or indicators” as used herein can refer to “biomarkers of degeneration, neuroinflammation or gliosis.”

The term “tau (t)” as used herein can broadly include various modified or aggregates forms, for example, including pathological tau and phosphorylated tau.

### Compounds of Formula (I)

In one aspect, the disclosure provides compounds and compositions and methods of use thereof. In one aspect, a compound of the disclosure is represented by Formula III:

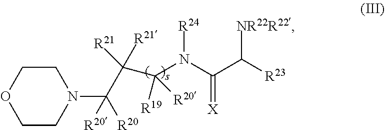

or a salt thereof, wherein:
X is CH₂, NH, O or S;
s is 0, 1, 2, 3 or 4;
each of R¹⁹, R^{19′}, R²⁰, R^{20′}, R²¹, R^{21′}, R²², R^{22′} and R²⁴ is independently selected at each occurrence from hydrogen and optionally substituted alkyl; or
R²⁰ and R^{20′} taken together form ═O, ═S, or =CH₂; or
R²⁰ and R²¹ taken together with the atoms to which they are attached form an optionally substituted cycloalkyl; or
R²⁰ and R²¹ taken together with the atoms to which they are attached form an optionally substituted aryl; or
R¹⁹ and R²⁰ taken together with the atoms to which they are attached form an optionally substituted cycloalkyl; or
R¹⁹ and R²⁰ taken together with the atoms to which they are attached form an optionally substituted aryl; and
   R²³ is hydrogen, optionally substituted alkyl, optionally substituted cycloalkyl or optionally substituted aryl.

In certain embodiments, for a compound or salt of Formula III, s is 0, 1 or 2. In an exemplary embodiment, s is 0.

In certain embodiments, for a compound or salt of Formula III, X is NH, 0 or S. In an exemplary embodiment, X is O.

For a compound or salt of Formula III, when R¹⁹, R^{19′}, R²⁰, R^{20′}, R²¹, R^{21′}, R²², R^{22′} or R²⁴ is optionally substituted, the optional substituents may be independently selected at each occurrence from halogen, —NO₂, —CN, —OR¹⁰⁰, —SR¹⁰⁰—N(R¹⁰⁰)₂, —S(═O)R¹⁰⁰, —S(═O)₂R¹⁰⁰, —S(═O)₂N(R¹⁰⁰)₂, —NR¹⁰⁰S(═O)₂R¹⁰⁰, —C(O)R¹⁰⁰, —C(O)OR¹⁰⁰, —OC(O)R¹⁰⁰, —OC(O)OR¹⁰⁰, —OC(O)N(R¹⁰⁰)₂, —NR¹⁰⁰C(O)R¹⁰⁰, —C(O)N(R¹⁰⁰)₂, ═O, ═S, ═N(R¹⁰⁰), —P(O)(OR¹⁰⁰)₂, —OP(O)(OR¹⁰⁰)₂; C₁₋₁₀ alkyl, C₂₋₁₀ alkenyl, and C₂₋₁₀ alkynyl, each of which is independently optionally substituted at each occurrence with one or more substituents selected from halogen, —NO₂, —CN, —OR¹⁰⁰, —SR¹⁰⁰—N(R¹⁰⁰)₂, —S(═O)R¹⁰⁰, —S(═O)₂R¹⁰⁰, —S(═O)₂N(R¹⁰⁰)₂, —NR¹⁰⁰S(═O)₂R¹⁰⁰, —C(O)R¹⁰⁰, —C(O)OR¹⁰⁰, —OC(O)R¹⁰⁰, —OC(O)OR¹⁰⁰, —OC(O)N(R¹⁰⁰)₂, —NR¹⁰⁰C(O)R¹⁰⁰, —C(O)N(R¹⁰⁰)₂, ═O, ═S, ═N(R¹⁰⁰), —P(O)(OR¹⁰⁰)₂, —OP(O)(OR¹⁰⁰)₂, C₃₋₁₂ carbocycle and 3- to 12-membered heterocycle; and C₃₋₁₂ carbocycle and 3- to 12-membered heterocycle, wherein each C₃₋₁₂ carbocycle and 3- to 12-membered heterocycle is independently optionally substituted with one or more substituents selected from halogen, —NO₂, —CN, —OR¹⁰⁰, —SR¹⁰⁰, —N(R¹⁰⁰)₂, —S(═O)R¹⁰⁰, —S(═O)₂R¹⁰⁰, —S(═O)₂N(R¹⁰⁰)₂, —NR¹⁰⁰S(═O)₂R¹⁰⁰, —C(O)R¹⁰⁰, —C(O)OR¹⁰⁰, —OC(O)R¹⁰⁰, —OC(O)OR¹⁰⁰, —OC(O)N(R¹⁰⁰)₂, —NR¹⁰C(O)R¹⁰⁰, —C(O)N(R¹⁰⁰)₂, ═O, ═S, ═N(R¹⁰⁰), —P(O)(OR¹⁰⁰)₂, —OP(O)(OR¹⁰⁰)₂, C₁₋₆ alkyl, C₁₋₆ haloalkyl, C₂₋₆ alkenyl, and C₂₋₆ alkynyl, wherein R¹⁰⁰ at each occurrence is independently selected from hydrogen; and C₁₋₂₀ alkyl, C₂₋₂₀ alkenyl, C₂₋₂₀ alkynyl, C₃₋₁₂ carbocycle and 3- to 12-membered heterocycle, each of which may be optionally substituted by halogen, —CN, —NO₂, —OH and —OCH₃.

For a compound or salt of Formula III, when R¹⁹, R^{19′}, R²⁰, R^{20′}, R²¹, R^{21′}, R²², R^{22′} or R²⁴ is optionally substituted, the optional substituents may be independently selected at each occurrence from halogen, —NO₂, —CN, —OR¹⁰⁰, —SR¹⁰⁰, —N(R¹⁰⁰)₂, —S(═O)R¹⁰⁰, —S(═O)₂R¹⁰⁰, —S(═O)₂N(R¹⁰⁰)₂, —NR¹⁰⁰S(═O)₂R¹⁰⁰, —C(O)R¹⁰⁰, —C(O)OR¹⁰⁰, —OC(O)R¹⁰⁰, —OC(O)OR¹⁰⁰, —OC(O)N(R¹⁰⁰)₂, —NR¹⁰⁰C(O)R¹⁰⁰, —C(O)N(R¹⁰⁰)₂, ═O, ═S, ═N(R¹⁰⁰), —P(O)(OR¹⁰⁰)₂, —OP(O)(OR¹⁰⁰)₂; C₁₋₁₀ alkyl optionally substituted at each occurrence with one or more substituents selected from halogen, —NO₂, —CN, —OR¹⁰⁰, —SR¹⁰⁰ and —N(R¹⁰⁰)₂; and C₃₋₁₂ carbocycle and 3- to 12-membered heterocycle, wherein each C₃₋₁₂ carbocycle and 3- to 12-membered heterocycle is independently optionally substituted with one or more substituents selected from halogen, —NO₂, —CN, —OR¹⁰⁰, —SR¹⁰⁰—N(R¹⁰⁰)₂, C₁₋₆ alkyl, and C₁₋₆ haloalkyl, wherein R¹⁰⁰ at each occurrence is independently selected from hydrogen; and C₁₋₂₀ alkyl, C₂₋₂₀ alkenyl, C₂₋₂₀ alkynyl, C₃₋₁₂ carbocycle and 3- to 12-membered heterocycle, each of which may be optionally substituted by halogen, —CN, —NO₂, —OH and —OCH₃.

In some embodiments, for a compound or salt of Formula III, s is selected from 1, 2, 3 or 4 and R¹⁹ and R^{19′} are independently selected at each occurrence from hydrogen and optionally substituted C₁-C₆ alkyl. In certain embodiments, s is selected from 1 or 2 and R¹⁹ and R^{19′} are independently selected at each occurrence from hydrogen and optionally substituted C₁-C₃ alkyl.

In certain embodiments, for a compound or salt of Formula III, R²⁰ and R^{20′} are independently selected from hydrogen and optionally substituted C₁-C₆ alkyl. In certain embodiments, R²⁰ and R^{20′} are independently selected from hydrogen and optionally substituted C₁-C₃ alkyl. In certain embodiments, R²⁰ and R^{20′} are each hydrogen.

In certain embodiments, for a compound or salt of Formula III, R²¹ and R^{21′} are independently selected from hydrogen and optionally substituted C₁-C₆ alkyl. In certain embodiments, R²¹ and R^{21′} are independently selected from hydrogen and optionally substituted C₁-C₃ alkyl. In certain embodiments, R²¹ and R^{21′} are each hydrogen.

In certain embodiments, for a compound or salt of Formula III, R²² and R^{22′} are independently selected from hydrogen and optionally substituted C₁-C₆ alkyl. In certain embodiments, R²² and R^{22′} are independently selected from hydrogen and optionally substituted C₁-C₃ alkyl. In certain embodiments, R²² and R^{22′} are each hydrogen.

In certain embodiments, for a compound or salt of Formula III, R²³ is selected from hydrogen, optionally substituted C₁-C₆ alkyl, optionally substituted cycloalkyl and optionally substituted aryl. In certain embodiments, R²³ is selected from optionally substituted C₁-C₆ alkyl, such as optionally substituted C₂-C₅ alkyl, such as optionally substituted C₃-C₅ alkyl, such as optionally substituted C₄ alkyl. In certain embodiments, R²³ is C₃-C₅ alkyl, such as C₄ alkyl. R²³ may be represented by the following structure:

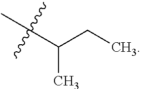 [SMILES: *C(C)CC]

In certain embodiments, for a compound or salt of Formula III, R²⁴ is hydrogen or optionally substituted C₁-C₆ alkyl alkyl. In certain embodiments, R²⁴ is selected from hydrogen and optionally substituted C₁-C₃ alkyl. In certain embodiments, R²⁴ is hydrogen.

In certain embodiments, for a compound or salt of Formula III, s is 0, X is O or S, R²⁰ and R^{20′} are independently selected from hydrogen and optionally substituted C₁-C₃ alkyl, R²¹ and R^{21′} are independently selected from hydrogen and optionally substituted C₁-C₃ alkyl, R²² and R^{22′} are independently selected from hydrogen and optionally substituted C₁-C₃ alkyl, R²³ is optionally substituted C₂-C₅ alkyl, and R²⁴ is selected from hydrogen and optionally substituted C₁-C₃ alkyl.

In certain embodiments, for a compound or salt of Formula III, s is 0, X is O or S, R²⁰ and R^{20′} are each hydrogen, R²¹ and R^{21′} are each hydrogen, R²² and R^{22′} are independently selected from hydrogen and optionally substituted C₁-C₃ alkyl, R²³ is optionally substituted C₂-C₅ alkyl, and R²⁴ is selected from hydrogen and optionally substituted C₁-C₃ alkyl.

In certain embodiments, for a compound or salt of Formula III, s is 0, X is 0, R²⁰ and R^{20′} are independently selected from hydrogen and optionally substituted C₁-C₃ alkyl, R²¹ and R^{21′} are independently selected from hydrogen and optionally substituted C₁-C₃ alkyl, R²² and R^{22′} are each hydrogen, R²³ is optionally substituted C₄ alkyl, and R²⁴ is hydrogen.

In certain embodiments, a compound of the disclosure is represented by Formula I:

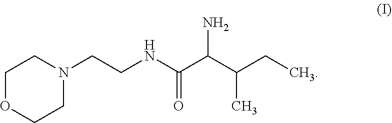 [SMILES: CCC(C)C(N)C(=O)NCCN1CCOCC1]

The compound of Formula I may be represented by Formula Ia:

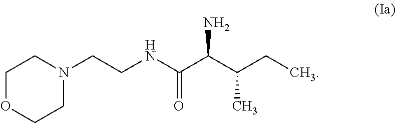 [SMILES: CC[C@H](C)[C@H](N)C(=O)NCCN1CCOCC1]

Included in the present disclosure are salts, particularly pharmaceutically acceptable salts, of the compounds of Formula (I) and (Ia). The compounds of the present invention can react with any of a number and inorganic and organic acids to form salts. In preferred embodiments, the compound of Formula (I) or (Ia) is an acid addition salt.

Acids commonly employed to form acid addition salts are inorganic acids such as hydrochloric acid, hydrobromic acid, hydroiodic acid, sulfuric acid, phosphoric acid, and the like, and organic acids such as p-toluenesulfonic acid, methanesulfonic acid, oxalic acid, p-bromophenyl-sulfonic acid, carbonic acid, succinic acid, citric acid, benzoic acid, acetic acid, and the like. Examples of such salts include the sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, sulfonate, xylenesulfonate, phenylacetate, phenylpropionate, phenylbutyrate, citrate, lactate, gamma-hydroxybutyrate, glycolate, tartrate, methanesulfonate, propanesulfonate, naphthalene-1-sulfonate, naphthalene-2-sulfonate, mandelate, and the like.

In certain embodiments, the salt of the compound of Formula (I) or (1a) is a sulfate salt, a bisulfate salt or a combination thereof.

In certain embodiments the salt of the compound of Formula (I) is represented by Formula (II):

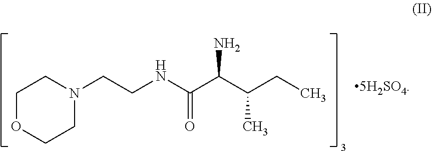

### Pharmaceutical Compositions

Pharmaceutical compositions may be formulated in a conventional manner using one or more physiologically acceptable carriers including excipients and auxiliaries which facilitate processing of the active compounds into preparations which can be used pharmaceutically. Proper formulation is dependent upon the route of administration chosen. Additional details about suitable excipients for pharmaceutical compositions described herein may be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure

Pharmaceutical compositions including a compound described herein may be manufactured in a conventional manner, such as, by way of example only, by means of conventional mixing, dissolving, granulating, dragee-making, levigating, emulsifying, encapsulating, entrapping or compression processes.

The pharmaceutical compositions will include at least one compound of Formulas (III), (I), (Ia), or (II) described herein, as an active ingredient in free-base form, or in a pharmaceutically acceptable salt form. In addition, the methods and pharmaceutical compositions described herein include the use of crystalline forms (also known as polymorphs), as well as active metabolites of these compounds having the same type of activity. In some situations, compounds may exist as tautomers. All tautomers are included within the scope of the compounds presented herein. Additionally, the compounds described herein can exist in unsolvated as well as solvated forms with pharmaceutically acceptable solvents such as water, ethanol, and the like. The solvated forms of the compounds presented herein are also included herein.

In certain embodiments, compositions provided herein may also include one or more preservatives to inhibit microbial activity. Suitable preservatives include quaternary ammonium compounds such as benzalkonium chloride, cetyltrimethylammonium bromide and cetylpyridinium chloride.

Pharmaceutical preparations for oral use can be obtained by mixing one or more solid excipients with one or more of the compounds described herein, e.g., compounds of Formulas (III), (I), (Ia), or (II), optionally grinding the resulting mixture, and processing the mixture of granules, after adding suitable auxiliaries, if desired, to obtain tablets, pills, or capsules. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents may be added, such as the cross-linked croscarmellose sodium, polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations that can be used orally include, for example, push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

In some embodiments, the solid dosage forms described herein may be in the form of a tablet, (including a suspension tablet, a fast-melt tablet, a bite-disintegration tablet, a rapid-disintegration tablet, an effervescent tablet, or a caplet), a pill, a powder (including a sterile packaged powder, a dispensable powder, or an effervescent powder), a capsule (including both soft or hard capsules, e.g., capsules made from animal-derived gelatin or plant-derived HPMC, or “sprinkle capsules”), solid dispersion, solid solution, bioerodible dosage form, controlled release formulations, pulsatile release dosage forms, multiparticulate dosage forms, pellets, granules, or an aerosol. In other embodiments, the pharmaceutical formulation is in the form of a powder. Additionally, pharmaceutical formulations of the compounds described herein may be administered as a single capsule or in multiple capsule dosage form. In some embodiments, the pharmaceutical composition is administered in two, or three, or four, capsules or tablets.

In some embodiments, solid dosage forms, e.g., tablets, effervescent tablets, and capsules, are prepared by mixing particles of a compound of Formulas (III), (I), (Ia), or (II) described herein, with one or more pharmaceutical excipients to form a bulk blend composition. When referring to these bulk blend compositions as homogeneous, it is meant that the particles of the compound of Formulas (III), (I), (Ia), or (II) described herein, are dispersed evenly throughout the composition so that the composition may be subdivided into equally effective unit dosage forms, such as tablets, pills, and capsules. The individual unit dosages may also include film coatings, which disintegrate upon oral ingestion or upon contact with diluent. These formulations can be manufactured by conventional pharmacological techniques.

In some embodiments the pharmaceutical composition may contain less than 10 wt % of excipients. In some embodiments the pharmaceutical composition may contain less than 5 wt % of excipients, such as less than 4 wt % of excipients, such as less than 3 wt % of excipients, such as less than 2 wt % of excipients. In certain embodiments, the pharmaceutical composition contains less than 2 wt % of excipients. In some embodiments, the pharmaceutical composition consists essentially of a compound of Formulas (III), (I), (Ia), or (II).

The disclosure provides for a pharmaceutical composition, wherein the composition comprises from about 10 milligram (mg) (free base weight) to about 1,000 mg (free base weight) of a salt of Formulas (III), (I), (Ia), or (II). The disclosure further provides for a pharmaceutical composition, wherein the composition comprises from about 10 mg (free base weight) to about 800 mg (free base weight) of a salt of Formulas (III), (I), (Ia), or (II). The disclosure further provides for a pharmaceutical composition, wherein the composition comprises from about 10 mg (free base weight) to about 600 mg (free base weight) of a salt of Formulas (III), (I), (Ia), or (II). The disclosure further provides for a pharmaceutical composition, wherein the composition comprises from about 10 mg (free base weight) to about 500 mg (free base weight) of a salt of Formulas (III), (I), (Ia), or (II).

As referred to herein, the “free base weight” refers to a calculated mass of the free base, based upon the mass of the salt in the composition. For example, to obtain a pharmaceutical composition with 200 mg (free base weight) of the salt of Formula (II), 334 mg of the salt of Formula (II) is added to the composition. The following equation may be used to calculate the weight of the free base from the weight of the salt of Formula (II):

$$( weight ( g ) of salt of Formula ( II ) ) \div ( MW ( g / mol ) of salt of formula ( II ) ) \times 3 \times ( MW of free base ) = weight of the free base$$

MW of salt of Formula (II):1220.43 g/mol
MW of free base (compound of Formula I): 243.35

### Methods of Treating or Managing Alzheimer's Disease

In certain embodiments the compounds and salts described herein are used for preventing, treating, ameliorating, managing, delaying onset, or slowing progression of (e.g., mild to moderate) Alzheimer's disease in a subject in need thereof. The compounds and salts described herein can be used to modulate p75 neurotrophin receptor activity, or for the treatment of diseases or conditions that would benefit, at least in part, from modulation of p75 neurotrophin receptor activity. In addition, a method for treating any of the diseases or conditions described herein in a subject in need of such treatment, may involve (e.g., oral) administration of pharmaceutical compositions containing at least one compound described herein, or a pharmaceutically acceptable salt, pharmaceutically acceptable prodrug, or pharmaceutically acceptable solvate thereof, in therapeutically effective amount(s) to the subject. The subject may exhibit or be determined to exhibit mild to moderate Alzheimer's disease according to McKhann (2011) criteria, e.g., within six months prior to the administration. In other cases, the subject may exhibit or be determined to be in preclinical or earlier clinical stages of the disease. The compositions containing the compound or salts described herein (e.g., a compound of Formulas (III), (I), (Ia), or (II) described herein) can be administered for prophylactic and/or therapeutic treatments. In therapeutic applications, the compositions are administered to a patient already suffering from a disease or condition, in an amount effective to treat or at least partially arrest the symptoms or underlying degeneration and progression of the disease or condition. Amounts effective for this use will depend on the severity and course of the disease or condition, previous therapy, the patient's health status, weight, and response to the drugs, and the judgment of the treating physician.

In prophylactic applications, including those intended to delay onset of clinical symptoms such as loss of cognitive function, compositions containing the compounds or salts described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like. When used in a patient, effective amounts for this use will depend on the severity and course of the disease, disorder or condition, previous therapy, the patient's health status and response to the drugs, and the judgment of the treating physician.

The amount of a given agent that will correspond to such an amount will vary depending upon factors such as the particular compound, disease or condition and its severity, the identity (e.g., weight) of the subject or host in need of treatment, but can nevertheless be determined in a manner recognized in the field according to the particular circumstances surrounding the case, including, e.g., the specific agent being administered, the route of administration, the condition being treated, and the subject or host being treated. In certain aspects, the pharmaceutical composition is administered in the morning and the evening. The pharmaceutical composition may be administered to said subject in a fed or fasted state. The pharmaceutical composition may be administered daily, e.g., once or more than once daily, such as twice or more times daily. The pharmaceutical composition may be administered for a period of two or more weeks, three or more weeks, four or more weeks, five or more weeks, six or more weeks, seven or more weeks, two or more months, three or more months, four or more months, five or more months, or six or more months, or one or more years, or one or more decades. In mouse PK-PD studies, twice per day dosing tends to give greater efficacy than once per day dosing.

Suitable unit dosage forms for oral administration include from about 1 to about 1,000 mg active ingredient (free base weight). In one embodiment, the unit dosage is about 1 to about 800 mg, about 1 to about 600 mg, or about 1 to about 500 mg, (all weights of free base). In one embodiment, the unit dosage is about 10 to about 1,000 mg, about 10 to about 800 mg, about 10 to about 600 mg, or about 10 to about 500 mg, (all weights of free base). The foregoing ranges are merely suggestive, as the number of variables in regard to an individual treatment regime is large, and considerable excursions from these recommended values are not uncommon. Such dosages may be altered depending on a number of variables, not limited to the activity of the compound used, the disease or condition to be treated, the response of imaging or cerebrospinal based biomarkers, the mode of administration, the requirements of the individual subject, the severity of the disease or condition being treated, and the judgment of the practitioner.

In some embodiments, the pharmaceutical composition comprises from about 10 milligram (mg) (free base weight) to about 1000 mg (free base weight) of the salt of the compound of Formula (I). In some embodiments, the pharmaceutical composition comprises from about 10 milligram (mg) (free base weight) to about 900 mg (free base weight) of the salt of the compound of Formula (I). In some embodiments, the pharmaceutical composition comprises from about 10 milligram (mg) (free base weight) to about 800 mg (free base weight) of the salt of the compound of Formula (I). In some embodiments, the pharmaceutical composition comprises from about 10 milligram (mg) (free base weight) to about 600 mg (free base weight) of the salt of the compound of Formula (I). In some embodiments, the pharmaceutical composition comprises from about 10 milligram (mg) (free base weight) to about 500 mg (free base weight) of the salt of the compound of Formula (I). In some embodiments, the pharmaceutical composition comprises from about 10 milligram (mg) (free base weight) to about 400 mg (free base weight) of the salt of the compound of Formula (I). In some embodiments, the pharmaceutical composition comprises from about 10 milligram (mg) (free base weight) to about 300 mg (free base weight) of the salt of the compound of Formula (I). In some embodiments, the pharmaceutical composition comprises from about 10 milligram (mg) (free base weight) to about 200 mg (free base weight) of the salt of the compound of Formula (I). In some embodiments, the pharmaceutical composition comprises from about 10 milligram (mg) (free base weight) to about 100 mg (free base weight) of the salt of the compound of Formula (I).

In some embodiments, the p75NTR receptor modulator or the pharmaceutically acceptable salt thereof, or the pharmaceutical composition, is formulated for administration at a dose of 100 mg b.i.d. (bis in die, twice a day). In some embodiments, the p75NTR receptor modulator or the pharmaceutically acceptable salt thereof, or the pharmaceutical composition, is formulated for administration at a dose of 200 mg b.i.d. In some embodiments, the p75NTR receptor modulator or the pharmaceutically acceptable salt thereof, or the pharmaceutical composition, is formulated for administration at a dose of 300 mg b.i.d. In some embodiments, the p75NTR receptor modulator or the pharmaceutically acceptable salt thereof, or the pharmaceutical composition, is formulated for administration at a dose of 400 mg b.i.d. In some embodiments, the p75NTR receptor modulator or the pharmaceutically acceptable salt thereof, or the pharmaceutical composition, is formulated for administration at a dose of 100 mg b.i.d. to 800 mg b.i.d. In some embodiments, the p75NTR receptor modulator or the pharmaceutically acceptable salt thereof, or the pharmaceutical composition, is formulated for administration at a dose of 100 mg b.i.d. to 700 mg b.i.d. In some embodiments, the p75NTR receptor modulator or the pharmaceutically acceptable salt thereof, or the pharmaceutical composition, is formulated for administration at a dose of 100 mg b.i.d. to 600 mg b.i.d. In some embodiments, the p75NTR receptor modulator or the pharmaceutically acceptable salt thereof, or the pharmaceutical composition, is formulated for administration at a dose of 100 mg b.i.d. to 500 mg b.i.d. In some embodiments, the p75NTR receptor modulator or the pharmaceutically acceptable salt thereof, or the pharmaceutical composition, is formulated for administration at a dose of 100 mg b.i.d. to 400 mg b.i.d. In some embodiments, the p75NTR receptor modulator or the pharmaceutically acceptable salt thereof, or the pharmaceutical composition, is formulated for administration at a dose of 100 mg b.i.d. to 300 mg b.i.d.

In some embodiments of any one of the relevant methods described herein, the method described may be used to treat or prevent Alzheimer's disease in subjects with particular characteristics. For example, methods of the disclosure may be used to treat subjects who exhibit mild to moderate Alzheimer's disease according to McKhann (2011) criteria within six months prior to said administration. Other subjects might be in earlier stages such as preclinical or prodromal stages. Methods of the disclosure may be used to treat, delay onset or prevent Alzheimer's disease in subjects from 50 to 100 years old, such as from 50 to 90 years old, such as from 65 to 90 years old. Methods of the disclosure may be used to treat, delay onset, manage, or prevent Alzheimer's disease in subjects exhibiting or determined to exhibit an 84 allele of apolipoprotein E (ApoE) gene (ApoE4). Methods of the disclosure may be used to treat, manage, or prevent Alzheimer's disease in subjects not exhibiting or determined to not exhibit an 84 allele of apolipoprotein E (ApoE) gene (ApoE4).

In some embodiments of any one of the relevant methods described herein, the method described may provide or result in a slowing of deterioration of, in an improvement of, a value of one or more Alzheimer's disease metrics relative to a baseline value measured at or within six months (e.g., within three months) preceding said administration. The Alzheimer's disease metrics may be selected from anatomical or statistical regional or voxel-based brain glucose metabolism (¹⁸F-FDG-PET) rate, magnetic resonance imaging (MRI) structural or voxel-based or volumetric imaging, cerebrospinal Alzheimer's disease-relevant biomarker levels, blood or plasma biomarkers and performance on the cognitive testing methods such as ADAS-cog, MMSE and other cognitive testing approaches.

In some embodiments of any one of the relevant methods described herein, methods of the disclosure reduce or prevent loss of certain brain region volumes in said subject. In some embodiments, the brain region volume comprises one or more of a hippocampus volume, basal forebrain volume, lingual gyrus volume, parahippocampal gyrus, and orbitofrontal cortex, parietal cortex and cingulate cortex in said subject. In some embodiments volume of lateral ventricles in measured as an indicator of general brain volume; increased size of lateral ventricular volume is an indicator of diffuse loss of brain parenchymal volume. In some embodiments, the brain region volume comprises other brain region volumes in said subject. Such volumes could be derived from whole brain or targeted region voxel wise or voxel-based analyses.

In some embodiments of any one of the relevant methods described herein, methods of the disclosure reduce or prevent loss of hippocampus volume or loss or changes in other brain region or voxel-based volumes in said subject. In certain embodiments, methods of the disclosure reduce or prevent or mitigate an increase in certain species of amyloid beta (Aβ) level such as Aβ42 (e.g., in a bodily fluid, such as a cerebrospinal spinal fluid or blood). In certain embodiments, methods of the disclosure lead to a reduction in fluid levels of Aβ 42, 40 or other species. In certain embodiments, methods of the disclosure reduce or prevent or mitigate an increase in tau or modified versions of tau level (e.g., in a bodily fluid, such as a cerebrospinal spinal fluid or blood or plasma).

In certain embodiments, methods of the disclosure reduce or prevent or mitigate an increase in biomarkers of neuronal or synaptic degeneration such as neurofilament light chain, SNAP-25, synaptotagmin-1, neurogranin; or in biomarkers of inflammation or gliosis such as sTREM or YKL-40.

In some embodiments, the disclosure of the present application provides a method for modulating one or more neurodegeneration biomarkers or indicators in a subject in need thereof, the method comprising: administering to said subject an effective amount of a p75^{NTR} receptor modulator or a pharmaceutically acceptable salt thereof (such as described herein) to provide one or more of the following in said subject: (i) an amyloid beta (Aβ) level (e.g., in a bodily fluid) that is lower than a corresponding reference; (ii) a change (e.g., increase) or a rate of change (e.g., a rate of increase) in an Aβ level (e.g., in a bodily fluid) over a duration that is lower than a corresponding reference over the same duration; (iii) a change (e.g., increase) or a rate of change (e.g., a rate of increase) in a tau (T) level (e.g., in a bodily fluid) over a duration that is lower than a corresponding reference over the same duration; (iv) a change (e.g., increase) or a rate of change (e.g., a rate of increase) in a presynaptic biomarker level (e.g., in a bodily fluid) over a duration that is lower than a corresponding reference over the same duration; (v) a change (e.g., increase) or a rate of change (e.g., a rate of increase) in a postsynaptic biomarker level (e.g., in a bodily fluid) over a duration that is lower than a corresponding reference over the same duration; (vi) a change (e.g., increase) or a rate of change (e.g., a rate of increase) in a glial marker level (e.g., in a bodily fluid) over a duration that is lower than a corresponding reference over the same duration; (vii) a rate of volume change of a brain region over a duration that is lower than a corresponding reference rate (e.g., in an untreated control) over the same duration as determined by magnetic resonance imaging (MRI) imaging; (viii) a brain glucose metabolism (¹⁸F-FDG-PET) rate that is lower than a corresponding reference rate (e.g., in an untreated control); and (ix) a change in Alzheimer's Disease Assessment Scale-Cognitive (ADAS-Cog) score over a duration that is less than a corresponding change (e.g., in an untreated control) over the same duration.

In some embodiments, the disclosure of the present application provides a method for modulating one or more neurodegeneration biomarkers or indicators in a subject in need thereof, the method comprising: administering to said subject an effective amount of a p75^{NTR} receptor modulator or a pharmaceutically acceptable salt thereof to provide an amyloid beta (Aβ) level (e.g., in a bodily fluid) in said subject that is lower than a corresponding reference. In some embodiments, the Aβ is Aβ40 or Aβ42. In some embodiments, the Aβ is Aβ40. In some embodiments, the Aβ is Aβ42. In some embodiments, the method provides an Aβ level (e.g., in said bodily fluid) in said subject that is lower by at least about 1%, at least about 2%, at least about 3%, at least about 4%, or at least about 5% than said corresponding reference. In some embodiments, the corresponding reference is a corresponding pretreatment level said subject (e.g., in said bodily fluid). In some embodiments, the corresponding reference is determined from an untreated control.

In some embodiments, the disclosure of the present application provides a method for modulating one or more neurodegeneration biomarkers or indicators in a subject in need thereof, the method comprising: administering to said subject an effective amount of a p75^{NTR} receptor modulator or a pharmaceutically acceptable salt thereof to provide a change (e.g., increase) or a rate of change (e.g., a rate of increase) in an Aβ level (e.g., in a bodily fluid) over a duration that is lower than a corresponding reference over the same duration. In some embodiments, the Aβ is Aβ40 or Aβ42. In some embodiments, the Aβ is Aβ40. In some embodiments, the Aβ is Aβ42. In some embodiments, the method provides a change (e.g., increase) or a rate of change (e.g., a rate of increase) in an Aβ level (e.g., in a bodily fluid) over a duration that is lower by at least about 1%, at least about 2%, at least about 3%, at least about 4%, or at least about 5% than a corresponding reference over the same duration.

In some embodiments, the corresponding reference is a corresponding pretreatment level said subject (e.g., in said bodily fluid). In some embodiments, the corresponding reference is determined from an untreated control.

In some embodiments, the disclosure of the present application provides a method for modulating one or more neurodegeneration biomarkers or indicators in a subject in need thereof, the method comprising: administering to said subject an effective amount of a p75^{NTR} receptor modulator or a pharmaceutically acceptable salt thereof to provide a change (e.g., increase) or a rate of change (e.g., a rate of increase) in a tau (i) level (e.g., in a bodily fluid) over a duration that is lower than a corresponding reference over the same duration. In some embodiments, the tau comprises unphosphorylated tau. In some embodiments, the tau comprises phosphorylated tau. In some embodiments, the bodily fluid is a cerebrospinal spinal fluid, blood, serum, or plasma. In some embodiments, the bodily fluid is a cerebrospinal spinal fluid. In some embodiments, the bodily fluid is blood. In some embodiments, the bodily fluid is serum. In some embodiments, the bodily fluid is plasma. In some embodiments, the method provides a tau (i) level (e.g., in said bodily fluid) in said subject that as a rate of increase lower by at least about 1, at least about 2, at least about 3, at least about 4, or at least about 5 annual percent change (APC)) than said corresponding reference. In some embodiments, the corresponding reference is a corresponding pretreatment level (e.g., in said bodily fluid). In some embodiments, the corresponding reference is determined from an untreated control.

In some embodiments, the disclosure of the present application provides a method for modulating one or more neurodegeneration biomarkers or indicators in a subject in need thereof, the method comprising: administering to said subject an effective amount of a p75^{NTR} receptor modulator or a pharmaceutically acceptable salt thereof to provide a change (e.g., increase) or a rate of change (e.g., a rate of increase) in a presynaptic biomarker level (e.g., in a bodily fluid) over a duration that is lower than a corresponding reference over the same duration. In some embodiments, the presynaptic biomarker is selected from Synaptosome Associated Protein 25 (SNAP25), and Synaptotagmin 1 (SYT1). In some embodiments, the presynaptic biomarker is SNAP25. In some embodiments, the presynaptic biomarker is SYT1. In some embodiments, the bodily fluid is a cerebrospinal spinal fluid, blood, serum, or plasma. In some embodiments, the bodily fluid is a cerebrospinal spinal fluid. In some embodiments, the bodily fluid is blood. In some embodiments, the bodily fluid is serum. In some embodiments, the bodily fluid is plasma. In some embodiments, the method provides a presynaptic biomarker level (e.g., in said bodily fluid) in said subject that decreases at a slower rate than in an untreated control by at least about 1, at least about 2, at least about 3, at least about 4, or at least about 5 annual percent change (APC) or rendering a value that is lower than a corresponding reference. In some embodiments, the corresponding reference is a corresponding pretreatment level (e.g., in said bodily fluid). In some embodiments, the corresponding reference is determined from an untreated control.

In some embodiments, the disclosure of the present application provides a method for modulating one or more neurodegeneration biomarkers or indicators in a subject in need thereof, the method comprising: administering to said subject an effective amount of a p75^{NTR} receptor modulator or a pharmaceutically acceptable salt thereof to provide a change (e.g., increase) or a rate of change (e.g., a rate of increase) in a postsynaptic biomarker level (e.g., in a bodily fluid) over a duration that is lower than a corresponding reference over the same duration. In some embodiments, the postsynaptic biomarker is neurogranin (NG), such as neurogranin 36 (NG36). In some embodiments, the bodily fluid is a cerebrospinal spinal fluid, blood, serum, or plasma. In some embodiments, the bodily fluid is a cerebrospinal spinal fluid. In some embodiments, the bodily fluid is blood. In some embodiments, the bodily fluid is serum. In some embodiments, the bodily fluid is plasma. In some embodiments, the method provides a postsynaptic biomarker level (e.g., in said bodily fluid) in said subject that increases at a rate lower by at least about 1, at least about 2, at least about 3, at least about 4, or at least about 5 annual percent change (APC) than the corresponding reference. In some embodiments, the corresponding reference is a corresponding pretreatment level (e.g., in said bodily fluid). In some embodiments, the corresponding reference is determined from an untreated control.

In some embodiments, the disclosure of the present application provides a method for modulating one or more neurodegeneration biomarkers or indicators in a subject in need thereof, the method comprising: administering to said subject an effective amount of a p75^{NTR} receptor modulator or a pharmaceutically acceptable salt thereof to provide a change (e.g., increase) or a rate of change (e.g., a rate of increase) in a glial marker level (e.g., in a bodily fluid) over a duration that is lower than a corresponding reference over the same duration. In some embodiments, the glial marker is soluble Triggering Receptor Expressed on Myeloid cells 2 (sTREM2) or Chitinase 3-like 1 (CHI3L1, also called YKL40). In some embodiments, the glial marker is sTREM2. In some embodiments, the glial marker is YKL40. In some embodiments, the bodily fluid is a cerebrospinal spinal fluid, blood, serum, or plasma. In some embodiments, the bodily fluid is a cerebrospinal spinal fluid. In some embodiments, the bodily fluid is blood. In some embodiments, the bodily fluid is serum. In some embodiments, the bodily fluid is plasma. In some embodiments, the method provides a glial marker level (e.g., in said bodily fluid) in said subject that increases at a lower rate by at least about 1, at least about 2, at least about 3, at least about 4, or at least about 5 annual percent change (APC) than the corresponding reference. In some embodiments, the corresponding reference is a corresponding pretreatment level (e.g., in said bodily fluid). In some embodiments, the corresponding reference is determined from an untreated control.

In some embodiments, the disclosure of the present application provides a method for modulating one or more neurodegeneration biomarkers or indicators in a subject in need thereof, the method comprising: administering to said subject an effective amount of a p75^{NTR} receptor modulator or a pharmaceutically acceptable salt thereof to provide in said subject a rate of volume change of a brain region over a duration that is lower than a corresponding reference rate (e.g., in an untreated control) over the same duration as determined by magnetic resonance imaging (MRI) imaging. In some embodiments, the brain region comprises hippocampus. In some embodiments, the brain region comprises an anatomical brain region. In some embodiments, the brain region comprises the whole brain of the subject. In some embodiments, the brain region is determined by a voxel-based whole brain analysis. In some embodiments, the method provides a rate of volume change of said brain region that is lower by at least 1%, at least 2%, or at least 3% than said corresponding rate. In some embodiments, the corresponding reference rate is a corresponding pretreatment rate of volume change of said brain region. In some embodiments, the corresponding reference rate is determined from an untreated control.

In some embodiments, the disclosure of the present application provides a method for modulating one or more neurodegeneration biomarkers or indicators in a subject in need thereof, the method comprising: administering to said subject an effective amount of a p75^{NTR} receptor modulator or a pharmaceutically acceptable salt thereof to provide in said subject a brain glucose metabolism (¹⁸F-FDG-PET) rate (e.g., of decline) that is lower than a corresponding reference rate (e.g., of decline). In some embodiments, the ¹⁸F-FDG-PET rate is determined over a duration; and wherein said corresponding reference rate is determined over the same duration. In some embodiments, the corresponding reference is a corresponding pretreatment rate. In some embodiments, the corresponding reference is determined from an untreated control.

In some embodiments, the disclosure of the present application provides a method for modulating one or more neurodegeneration biomarkers or indicators in a subject in need thereof, the method comprising: administering to said subject an effective amount of a p75^{NTR} receptor modulator or a pharmaceutically acceptable salt thereof to provide in said subject a change in a neurological testing score over a duration that is less than a corresponding change over the same duration. In some embodiments, the neurological testing is selected from the Alzheimer's Disease Assessment Scale-Cognitive (ADAS-Cog), the Mini Mental Status Exam (MMSE), the Amunet spatial navigation testing, the Clinical Global Impression (CGI) scale, the Geriatric Depression Scale (GDS), the neurological testing battery (NTB), and combinations thereof. In some embodiments, the NTB is selected from the digit span test, the category fluency test, the controlled oral word association test (COWAT), the digit symbol substitution test (DSST), and combinations thereof. In some embodiments, the neurological testing is the Alzheimer's Disease Assessment Scale-Cognitive (ADAS-Cog). In some embodiments, the ADAS-Cog score is ADAS11 or ADAS13. In some embodiments, the ADAS-Cog score is ADAS11. In some embodiments, the ADAS-Cog score is ADAS13. In some embodiments, the corresponding reference is determined from an untreated control. In some embodiments, the corresponding reference change is a corresponding pretreatment change within a corresponding time period in said subject. In some embodiments, the time period is about three months. In some embodiments, the time period is about six months.

In certain embodiments, the disclosure provides for a method for modulating one or more neurodegeneration biomarkers or indicators in a subject in need thereof, the method comprising: administering to said subject an effective amount of a p75^{NTR} receptor modulator or a pharmaceutically acceptable salt thereof (e.g., for a time period (e.g., of at least six months)) to provide one or more of the following in said subject: (i) an amyloid beta (Aβ) level in a bodily fluid (e.g., a cerebrospinal fluid) that is lower than a corresponding reference (e.g., a corresponding pretreatment level in said bodily fluid); (ii) a tau (T) level in a bodily fluid (e.g., a cerebrospinal fluid) that is lower than a corresponding reference (e.g., a corresponding pretreatment level in said bodily fluid) or that increases at rate less than that expected in an untreated control; (iii) a presynaptic biomarker level in a bodily fluid (e.g., a cerebrospinal fluid) that is lower than a corresponding reference (e.g., a corresponding pretreatment level in said bodily fluid) or that increases at rate less than that expected in an untreated control; (iv) a postsynaptic biomarker level in a bodily fluid (e.g., a cerebrospinal fluid) that is lower than a corresponding reference (e.g., a corresponding pretreatment level in said bodily fluid) or that increases at rate less than that expected in an untreated control; (v) a glial marker level in a bodily fluid (e.g., a cerebrospinal fluid) that is lower than a corresponding reference (e.g., a corresponding pretreatment level in said bodily fluid) or that increases at rate less than that expected in an untreated control; (vi) a rate of volume change of a brain region (e.g., hippocampus) that is lower than a corresponding rate (e.g., a corresponding pretreatment rate of volume change of said brain region) or rate that would be expected to occur without treatment as determined by magnetic resonance imaging (MRI) imaging; (vii) a brain glucose metabolism (¹⁸F-FDG-PET) rate of decline that is lower than a corresponding reference (e.g., a corresponding pretreatment rate); and (viii) a change in Alzheimer's Disease Assessment Scale-Cognitive (ADAS-Cog) score within a time period (e.g., of about six months) that is less than a corresponding change (e.g., a corresponding pretreatment change within a corresponding time period in said subject). As described herein, a p75^{NTR} receptor modulator exhibits a binding specificity for p75^{NTR} receptor. In some embodiments, the pre-treatment level may be determined one or more days prior to the initial administration described herein. In some embodiments, the pre-treatment level may be determined less than 24 hours prior to the initial administration described herein. In some embodiments, any one or more members of items (i)-(viii) may be determined at one or more time point(s) before or after an initial administering or the first dose. In some embodiments, any one or more members of items (i)-(viii) may be determined at one or more time point(s) after a subsequent administering or a subsequent dose. In some embodiments, any one or more members of items (i)-(viii) may be determined at one or more time point(s) after a set of administering or a chronic dosing after a time period (e.g., of at least about 3, at least about 4, at least about 5, or at least about 6 months). In some embodiments, the method described herein maintains, prevents a significant decrease in, or effects an increase in, the Alzheimer's Disease Assessment Scale-Cognitive (ADAS-Cog) score of the subject, e.g., as compared with a pre-treatment level. In many cases, for a given subject, rates of change of fluid biomarker or imaging measures are not specifically determined for that individual. Instead, treatment is started empirically as the assumption that the treatment will slow rates of progression of degeneration and its various measures based on previous trial data and based on the profile of the subject matching that of trial participants.

In some embodiments of any one of the relevant methods described herein, the cerebrospinal (e.g., fluid) Alzheimer's disease-related biomarker levels include the levels (or certain ratios thereof) of one or more of tau, p-tau, Aβ40, Aβ42, AchE, neurofilaments light chain (an indicator of neuronal degeneration), SNAP-25 (an indicator of neuronal degeneration, especially pre-synaptic degeneration), neurogranin (an indicator of neuronal degeneration, especially post-synaptic degeneration), synaptotagmin-1 (an indicator of neuronal degeneration, especially pre-synaptic degeneration), sTREM (an indicator of neuroinflammation or gliosis) and YKL-40 (an indicator of neuroinflammation or gliosis; or any ratios thereof such as Ab 42/40.). In some embodiments, the cerebrospinal Alzheimer's disease-related biomarker levels include the levels of one or more of tau, p-tau, Aβ40, Aβ42, AchE activity. In some embodiments, the cerebrospinal Alzheimer's disease-related biomarker levels include the levels of one or more of indicators of neuronal degeneration such as neurofilaments light chain, SNAP-25, neurogranin, and synaptotagmin-1. In some embodiments, the cerebrospinal Alzheimer's disease-related biomarker levels include the levels of one or more of indicators of neuroinflammation or gliosis such as sTREM and YKL-40. In some embodiments, the cerebrospinal Alzheimer's disease-related biomarker is selected from any one or any subset of those set forth in Table A. Many of these biomarkers can also be assessed in blood or plasma samples

TABLE A
List of cerebrospinal Alzheimer's Disease-related biomarkers
tau
p-tau
Aβ40
Aβ42
AchE
neurofilaments light chain
SNAP-25
Neurogranin
synaptotagmin-1
sTREM
YKL-40

In some embodiments of any one of the relevant methods described herein, the cognitive testing methods include one or more of the Alzheimer's disease assessment scale (ADAS-cog), mini mental status exam (MMSE), Clinical Global Impression (CGI) scale, neurological testing battery (NTB), Spatial navigation Testing with Amunet, the Geriatric Depression Scale (GDS), and other tests. In some embodiments, the cognitive testing methods include one or more of ADAS-cog and MMSE. In some embodiments, the cognitive testing methods include one or more of CGI, Spatial navigation Testing with Amunet, CGI scale, and NTB. The NTB may include one or more of the digit span test, the category fluency test, the controlled oral word association test (COWAT), and the digit symbol substitution test (DSST). In some embodiments, the cognitive testing method is selected from any one or any subset of those set forth in Table B.

TABLE B
List of cognitive testing methods

```
Alzheimer's disease assessment scal e(ADAS-cog)
Mini Mental Status Exam (MMSE)
Clinical Global Impression (CGI) scale
Neurological Testing Battery (NTB)
  Digit span test
  Category fluency test
  Controlled oral word association test (COWAT)
  Digit symbol substitution test (DSST)
```

In some embodiments of any one of the relevant methods described herein, the bodily fluid is a cerebrospinal spinal fluid, blood, serum, or plasma. In some embodiments, the bodily fluid is a cerebrospinal spinal fluid. In some embodiments, the bodily fluid is blood. In some embodiments, the bodily fluid is serum. In some embodiments, the bodily fluid is plasma.

In some embodiments of any one of the relevant methods described herein, the corresponding reference (e.g., level, value, rate, etc.) is a corresponding pretreatment level, value, rate (etc.) of the subject (e.g., in said bodily fluid). In some embodiments, the corresponding reference (e.g., level, value, rate, etc.) is determined from an untreated control (e.g., a corresponding subject or a placebo cohort).

In some embodiments of any one of the relevant methods described herein, the duration or time period (over which a change or a rate of change, for example, an increase or a rate of increase, in the one or more neurodegeneration biomarkers or indicators is determined) is at least about 5 days, at least about 10 days, at least about 15 days, at least about 20 days, at least about 25 days, at least about 30 days, at least about 1 month, at least about 2 months, at least about 3 months, at least about 4 months, at least about 5 months, or at least about 6 months. In some embodiments, the duration or time period is at least about 5 days. In some embodiments, the duration or time period is at least about 10 days. In some embodiments, the duration or time period is at least about 15 days. In some embodiments, the duration or time period is at least about 20 days. In some embodiments, the duration or time period is at least about 25 days. In some embodiments, the duration or time period is at least about 30 days. In some embodiments, the duration or time period is at least about 1 month. In some embodiments, the duration or time period is at least about 2 months. In some embodiments, the duration or time period is at least about 3 months. In some embodiments, the duration or time period is at least about 4 months. In some embodiments, the duration or time period is at least about 5 months. In some embodiments, the duration or time period is at least about 6 months.

In some embodiments of any one of the relevant methods described herein, the duration or time period (over which a change or a rate of change, for example, an increase or a rate of increase, in the one or more neurodegeneration biomarkers or indicators is determined) is from about 5 days to about 6 months, from about 10 days to about 6 months, from about 15 days to about 6 months, from about 20 days to about 6 months, from about 25 days to about 6 months, from about 30 days to about 6 months, from about 1 month to about 6 months, from about 2 months to about 6 months, from about 3 months to about 6 months, from about 4 months to about 6 months, or from about 5 months to about 6 months. In some embodiments, the duration or time period is from about 5 days to about 6 months. In some embodiments, the duration or time period is from about 10 days to about 6 months. In some embodiments, the duration or time period is from about 15 days to about 6 months. In some embodiments, the duration or time period is from about 20 days to about 6 months. In some embodiments, the duration or time period is from about 25 days to about 6 months. In some embodiments, the duration or time period is from about 30 days to about 6 months. In some embodiments, the duration or time period is from about 1 month to about 6 months. In some embodiments, the duration or time period is from about 2 months to about 6 months. In some embodiments, the duration or time period is from about 3 months to about 6 months. In some embodiments, the duration or time period is from about 4 months to about 6 months. In some embodiments, the duration or time period is from about 5 months to about 6 months.

In some embodiments of any one of the relevant methods described herein, said administering is performed more than once daily. In some embodiments, said administering is performed twice daily. In some embodiments, said administering is performed three times daily. In some embodiments, said administering is orally. In some embodiments, said administering comprises administering to said subject said effective amount of said p75_{NTR} receptor modulator or said pharmaceutically acceptable salt thereof for a duration of at least three months. In some embodiments, said administering comprises administering to said subject said effective amount of said p75^{NTR} receptor modulator or said pharmaceutically acceptable salt thereof for a duration of at least six months.

In some embodiments of any one of the relevant methods described herein, the subject exhibits or is determined to exhibit mild to moderate Alzheimer's disease according to McKhann (2011) criteria within six months prior to said administration. In some embodiments, the subject exhibits or is determined to exhibit mild to moderate Alzheimer's disease according to McKhann (2011) criteria within three months prior to said administration. In some embodiments, the subject exhibits or is determined to be at high risk or in a pre-clinical or prodromal state of Alzheimer's disease as determined by genetic risk and/or biomarkers within six months prior to said administration. In some embodiments, the subject exhibits or is determined to be at high risk or in a pre-clinical or prodromal state of Alzheimer's disease as determined by genetic risk and/or biomarkers within three months prior to said administration. In some embodiments, the subject exhibits or is determined to exhibit an ε4 allele of apolipoprotein E (ApoE) gene. In some embodiments, the subject exhibits or is determined to exhibit no ε4 allele of apolipoprotein E (ApoE) gene. In some embodiments, the subject is between the ages of 50 and 90. In some embodiments, the subject is younger than about 72 years of age. In some embodiments, the subject is of about 72 years of age or older.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### LIST OF EMBODIMENTS

The following list of embodiments of the invention are to be considered as disclosing various features of the invention, which features can be considered to be specific to the particular embodiment under which they are discussed, or which are combinable with the various other features as listed in other embodiments. Thus, simply because a feature is discussed under one particular embodiment does not necessarily limit the use of that feature to that embodiment.

Embodiment 1. A method for modulating one or more neurodegeneration biomarkers or indicators in a subject in need thereof, the method comprising: administering to said subject an effective amount of a p75^{NTR} receptor modulator or a pharmaceutically acceptable salt thereof to provide one or more of the following in said subject: (i) an amyloid beta (Aβ) level (e.g., in a bodily fluid) that is lower than a corresponding reference; (ii) a change (e.g., increase) or a rate of change (e.g., a rate of increase) in an Aβ level (e.g., in a bodily fluid) over a duration that is lower than a corresponding reference over the same duration; (iii) a change (e.g., increase) or a rate of change (e.g., a rate of increase) in a tau (T) level (e.g., in a bodily fluid) over a duration that is lower than a corresponding reference over the same duration; (iv) a change (e.g., increase) or a rate of change (e.g., a rate of increase) in a presynaptic biomarker level (e.g., in a bodily fluid) over a duration that is lower than a corresponding reference over the same duration; (v) a change (e.g., increase) or a rate of change (e.g., a rate of increase) in a postsynaptic biomarker level (e.g., in a bodily fluid) over a duration that is lower than a corresponding reference over the same duration; (vi) a change (e.g., increase) or a rate of change (e.g., a rate of increase) in a glial marker level (e.g., in a bodily fluid) over a duration that is lower than a corresponding reference over the same duration; (vii) a rate of volume change of a brain region over a duration that is lower than a corresponding reference rate (e.g., in an untreated control) over the same duration as determined by magnetic resonance imaging (MRI) imaging; (viii) a brain glucose metabolism (¹⁸F-FDG-PET) rate that is lower than a corresponding reference rate (e.g., in an untreated control); and (ix) a change in Alzheimer's Disease Assessment Scale-Cognitive (ADAS-Cog) score over a duration that is less than a corresponding change (e.g., in an untreated control) over the same duration.

Embodiment 2. A method for modulating one or more neurodegeneration biomarkers or indicators in a subject in need thereof, the method comprising: administering to said subject an effective amount of a p75^{NTR} receptor modulator or a pharmaceutically acceptable salt thereof to provide an amyloid beta (Aβ) level (e.g., in a bodily fluid) in said subject that is lower than a corresponding reference.

Embodiment 3. A method for modulating one or more neurodegeneration biomarkers or indicators in a subject in need thereof, the method comprising: administering to said subject an effective amount of a p75^{NTR} receptor modulator or a pharmaceutically acceptable salt thereof to provide a change (e.g., increase) or a rate of change (e.g., a rate of increase) in an Aβ level (e.g., in a bodily fluid) over a duration that is lower than a corresponding reference over the same duration.

Embodiment 4. The method of embodiment 1 or 3, wherein the duration is at least about 5 days, at least about 10 days, at least about 15 days, at least about 20 days, at least about 25 days, at least about 30 days, at least about 1 month, at least about 2 months, at least about 3 months, at least about 4 months, at least about 5 months, or at least about 6 months.

Embodiment 5. The method of any one of embodiments 1-4, wherein said Aβ is Aβ40 or Aβ42.

Embodiment 6. The method of embodiment 5, wherein said Aβ is Aβ40.

Embodiment 7. The method of embodiment 5, wherein said Aβ is Aβ42.

Embodiment 8. The method of any one of embodiments 1-7, wherein said bodily fluid is a cerebrospinal spinal fluid, blood or plasma.

Embodiment 9. The method of any one of embodiments 1-8, wherein the method provides an Aβ level in said bodily fluid in said subject that is lower by at least about 1%, at least about 2%, at least about 3%, at least about 4%, or at least about 5% than said corresponding reference.

Embodiment 10. The method of any one of embodiments 1-9, wherein said corresponding reference is a corresponding pretreatment level in said bodily fluid.

Embodiment 11. The method of any one of embodiments 1-9, wherein said corresponding reference is determined from an untreated control.

Embodiment 12. A method for modulating one or more neurodegeneration biomarkers or indicators in a subject in need thereof, the method comprising: administering to said subject an effective amount of a p75^{NTR} receptor modulator or a pharmaceutically acceptable salt thereof to provide a change (e.g., increase) or a rate of change (e.g., a rate of increase) in a tau (T) level (e.g., in a bodily fluid) over a duration that is lower than a corresponding reference over the same duration.

Embodiment 13. The method of embodiment 1 or 12, wherein the duration is at least about 5 days, at least about 10 days, at least about 15 days, at least about 20 days, at least about 25 days, at least about 30 days, at least about 1 month, at least about 2 months, at least about 3 months, at least about 4 months, at least about 5 months, or at least about 6 months.

Embodiment 14. The method of any one of embodiments 1 and 12-13, wherein said tau comprises phosphorylated tau.

Embodiment 15. The method of any one of embodiments 1 and 12-14, wherein said bodily fluid is a cerebrospinal spinal fluid, blood or plasma.

Embodiment 16. The method of any one of embodiments 1 and 12-15, wherein said method provides a tau (i) level in said bodily fluid in said subject that as a rate of increase lower by at least about 1, at least about 2, at least about 3, at least about 4, or at least about 5 annual percent change (APC)) than said corresponding reference.

Embodiment 17. The method of any one of embodiments 1 and 12-16, wherein said corresponding reference is a corresponding pretreatment level in said bodily fluid.

Embodiment 18. The method of any one of embodiments 1 and 12-16, wherein said corresponding reference is determined from an untreated control.

Embodiment 19. A method for modulating one or more neurodegeneration biomarkers or indicators in a subject in need thereof, the method comprising: administering to said subject an effective amount of a p75^{NTR} receptor modulator or a pharmaceutically acceptable salt thereof to provide a change (e.g., increase) or a rate of change (e.g., a rate of increase) in a presynaptic biomarker level (e.g., in a bodily fluid) over a duration that is lower than a corresponding reference over the same duration.

Embodiment 20. The method of embodiment 1 or 19, wherein the duration is at least about 5 days, at least about 10 days, at least about 15 days, at least about 20 days, at least about 25 days, at least about 30 days, at least about 1 month, at least about 2 months, at least about 3 months, at least about 4 months, at least about 5 months, or at least about 6 months.

Embodiment 21. The method of any one of embodiments 1 and 19-20, wherein said presynaptic biomarker is selected from Synaptosome Associated Protein 25 (SNAP25), and Synaptotagmin 1 (SYT1).

Embodiment 22. The method of embodiment 21, wherein said presynaptic biomarker is SNAP25.

Embodiment 23. The method of embodiment 21, wherein said presynaptic biomarker is SYT1.

Embodiment 24. The method of any one of embodiments 1 and 19-23, wherein said bodily fluid is a cerebrospinal spinal fluid, blood or plasma.

Embodiment 25. The method of any one of embodiments 1 and 19-24, wherein the method provides a presynaptic biomarker level in said bodily fluid in said subject that decreases at a slower rate than in an untreated control by at least about 1, at least about 2, at least about 3, at least about 4, or at least about 5 annual percent change (APC) or rendering a value that is lower than a corresponding reference.

Embodiment 26. The method of any one of embodiments 1 and 19-25, wherein said corresponding reference is a corresponding pretreatment level in said bodily fluid.

Embodiment 27. The method of any one of embodiments 1 and 19-25, wherein said corresponding reference is determined from an untreated control.

Embodiment 28. A method for modulating one or more neurodegeneration biomarkers or indicators in a subject in need thereof, the method comprising: administering to said subject an effective amount of a p75^{NTR} receptor modulator or a pharmaceutically acceptable salt thereof to provide a change (e.g., increase) or a rate of change (e.g., a rate of increase) in a postsynaptic biomarker level (e.g., in a bodily fluid) over a duration that is lower than a corresponding reference over the same duration.

Embodiment 29. The method of embodiment 1 or 28, wherein the duration is at least about 5 days, at least about 10 days, at least about 15 days, at least about 20 days, at least about 25 days, at least about 30 days, at least about 1 month, at least about 2 months, at least about 3 months, at least about 4 months, at least about 5 months, or at least about 6 months.

Embodiment 30. The method of any one of embodiments 1 and 28-29, wherein said postsynaptic biomarker is neurogranin (NG).

Embodiment 31. The method of any one of embodiments 1 and 28-30, wherein said bodily fluid is a cerebrospinal spinal fluid, blood or plasma.

Embodiment 32. The method of any one of embodiments 1 and 28-31, wherein the method provides a postsynaptic biomarker level in said bodily fluid in said subject that increases at a rate lower by at least about 1, at least about 2, at least about 3, at least about 4, or at least about 5 annual percent change (APC) than the corresponding reference.

Embodiment 33. The method of any one of embodiments 1 and 28-32, wherein said corresponding reference is a corresponding pretreatment level in said bodily fluid.

Embodiment 34. The method of any one of embodiments 1 and 28-32, wherein said corresponding reference is determined from an untreated control.

Embodiment 35. A method for modulating one or more neurodegeneration biomarkers or indicators in a subject in need thereof, the method comprising: administering to said subject an effective amount of a p75^{NTR} receptor modulator or a pharmaceutically acceptable salt thereof to provide a change (e.g., increase) or a rate of change (e.g., a rate of increase) in a glial marker level (e.g., in a bodily fluid) over a duration that is lower than a corresponding reference over the same duration.

Embodiment 36. The method of embodiment 1 or 35, wherein the duration is at least about 5 days, at least about 10 days, at least about 15 days, at least about 20 days, at least about 25 days, at least about 30 days, at least about 1 month, at least about 2 months, at least about 3 months, at least about 4 months, at least about 5 months, or at least about 6 months.

Embodiment 37. The method of any one of embodiments 1 and 35-36, wherein said glial marker is soluble Triggering Receptor Expressed on Myeloid cells 2 (sTREM2) or Chitinase 3-like 1 (CHI3L1, also called YKL40).

Embodiment 38. The method of embodiment 37, wherein said glial marker is sTREM2.

Embodiment 39. The method of embodiment 37, wherein said glial marker is YKL40.

Embodiment 40. The method of any one of embodiments 1 and 35-39, wherein said bodily fluid is a cerebrospinal spinal fluid, blood or plasma.

Embodiment 41. The method of any one of embodiments 1 and 35-40, wherein the method provides a glial marker level in said bodily fluid in said subject that increases at a lower rate by at least about 1, at least about 2, at least about 3, at least about 4, or at least about 5 annual percent change (APC) than the corresponding reference.

Embodiment 42. The method of any one of embodiments 1 and 35-41, wherein said corresponding reference is a corresponding pretreatment level in said bodily fluid.

Embodiment 43. The method of any one of embodiments 1 and 35-41, wherein said corresponding reference is determined from an untreated control.

Embodiment 44. A method for modulating one or more neurodegeneration biomarkers or indicators in a subject in need thereof, the method comprising: administering to said subject an effective amount of a p75^{NTR} receptor modulator or a pharmaceutically acceptable salt thereof to provide in said subject a rate of volume change of a brain region over a duration that is lower than a corresponding reference rate (e.g., in an untreated control) over the same duration as determined by magnetic resonance imaging (MRI) imaging.

Embodiment 45. The method of embodiment 1 or 44, wherein the duration is at least about 5 days, at least about 10 days, at least about 15 days, at least about 20 days, at least about 25 days, at least about 30 days, at least about 1 month, at least about 2 months, at least about 3 months, at least about 4 months, at least about 5 months, or at least about 6 months.

Embodiment 46. The method of any one of embodiments 1 and 44-45, wherein said brain region comprises hippocampus.

Embodiment 47. The method of any one of embodiments 1 and 44-45, wherein said brain region comprises an anatomical brain region.

Embodiment 48. The method of any one of embodiments 1 and 44-45, wherein said brain region comprises the whole brain of the subject.

Embodiment 49. The method of embodiment 48, wherein said brain region is determined by a voxel-based whole brain analysis.

Embodiment 50. The method of any one of embodiments 1 and 44-49, wherein the method provides a rate of volume change of said brain region that is lower by at least 1%, at least 2%, or at least 3% than said corresponding rate.

Embodiment 51. The method of any one of embodiments 1 and 44-50, wherein said corresponding reference rate is a corresponding pretreatment rate of volume change of said brain region.

Embodiment 52. The method of any one of embodiments 1 and 44-50, wherein said corresponding reference rate is determined from an untreated control.

Embodiment 53. A method for modulating one or more neurodegeneration biomarkers or indicators in a subject in need thereof, the method comprising: administering to said subject an effective amount of a p75^{NTR} receptor modulator or a pharmaceutically acceptable salt thereof to provide in said subject a brain glucose metabolism (¹⁸F-FDG-PET) rate (e.g., of decline) that is lower than a corresponding reference rate (e.g., of decline).

Embodiment 54. The method of embodiment 1 or 53, wherein said ¹⁸F-FDG-PET rate is determined over a duration; and wherein said corresponding reference rate is determined over the same duration.

Embodiment 55. The method of embodiment 1 or 54, wherein the duration is at least about 5 days, at least about 10 days, at least about 15 days, at least about 20 days, at least about 25 days, at least about 30 days, at least about 1 month, at least about 2 months, at least about 3 months, at least about 4 months, at least about 5 months, or at least about 6 months.

Embodiment 56. The method of 1 and 53-55, wherein said corresponding reference is a corresponding pretreatment rate.

Embodiment 57. The method of 1 and 53-55, wherein said corresponding reference is determined from an untreated control.

Embodiment 58. A method for modulating one or more neurodegeneration biomarkers or indicators in a subject in need thereof, the method comprising: administering to said subject an effective amount of a p75^{NTR} receptor modulator or a pharmaceutically acceptable salt thereof to provide in said subject a change in a neurological testing score over a duration that is less than a corresponding change over the same duration.

Embodiment 59. The method of embodiment 58, wherein the neurological testing is selected from the Alzheimer's Disease Assessment Scale-Cognitive (ADAS-Cog), the Mini Mental Status Exam (MMSE), the Amunet spatial navigation testing, the Clinical Global Impression (CGI) scale, the Geriatric Depression Scale (GDS), the neurological testing battery (NTB), and combinations thereof.

Embodiment 60. The method of embodiment 59, wherein the NTB is selected from the digit span test, the category fluency test, the controlled oral word association test (COWAT), the digit symbol substitution test (DSST), and combinations thereof.

Embodiment 61. The method of embodiment 58, wherein the neurological testing is the Alzheimer's Disease Assessment Scale-Cognitive (ADAS-Cog).

Embodiment 62. The method of any one of embodiments 1, 58 and 61, wherein the duration is at least about 5 days, at least about 10 days, at least about 15 days, at least about 20 days, at least about 25 days, at least about 30 days, at least about 1 month, at least about 2 months, at least about 3 months, at least about 4 months, at least about 5 months, or at least about 6 months.

Embodiment 63. The method of any one of embodiments 1, 58 and 61-62, wherein said ADAS-Cog score is ADAS11 or ADAS13.

Embodiment 64. The method of embodiment 63, wherein said ADAS-Cog score is ADAS11.

Embodiment 65. The method of embodiment 63, wherein said ADAS-Cog score is ADAS13.

Embodiment 66. The method of any one of embodiments 1 and 58-65, wherein said corresponding reference is determined from an untreated control.

Embodiment 67. The method of any one of embodiments 1 and 58-65, wherein said corresponding change is a corresponding pretreatment change within a corresponding time period in said subject.

Embodiment 68. The method of embodiment 66, wherein said time period is about three months.

Embodiment 69. The method of embodiment 66, wherein said time period is about six months.

Embodiment 70. The method of any one of embodiments 1 and 58-69, wherein said corresponding reference is a baseline value or rate of decline measured within six months preceding said administration.

Embodiment 71. The method of any one of embodiments 1 and 58-69, wherein said corresponding reference is a baseline value or rate of decline measured within three months preceding said administration.

Embodiment 72. The method of any one of embodiments 1-71, wherein said p75^{NTR} receptor modulator or said pharmaceutically acceptable salt thereof is a compound represented by the formula,

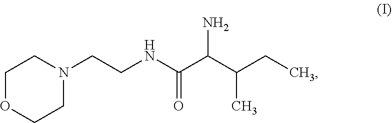 [SMILES: CCC(C)C(N)C(=O)NCCN1CCOCC1]

or a pharmaceutically acceptable salt thereof.

Embodiment 73. The method of embodiment 72, wherein the salt of the compound of Formula (I) is represented by Formula (II):

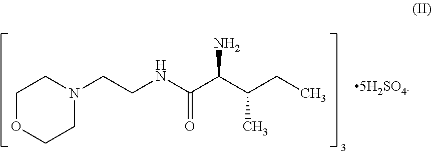

Embodiment 74. The method of any one of embodiments 1-73, wherein said effective amount of said p75^{NTR} receptor modulator or said pharmaceutically acceptable salt thereof is from about 10 milligram (mg) (free base weight) to about 1000 mg (free base weight).

Embodiment 75. The method of any one of embodiments 1-73, wherein said effective amount of said p75^{NTR} receptor modulator or said pharmaceutically acceptable salt thereof is from about 10 milligram (mg) (free base weight) to about 800 mg (free base weight).

Embodiment 76. The method of any one of embodiments 1-73, wherein said effective amount of said p75^{NTR} receptor modulator or said pharmaceutically acceptable salt thereof is from about 10 milligram (mg) (free base weight) to about 600 mg (free base weight).

Embodiment 77. The method of any one of embodiments 1-73, wherein said effective amount of said p75^{NTR} receptor modulator or said pharmaceutically acceptable salt thereof is from about 10 milligram (mg) (free base weight) to about 500 mg (free base weight).

Embodiment 78. The method of any one of embodiments 1-73, wherein said effective amount of said p75^{NTR} receptor modulator or said pharmaceutically acceptable salt thereof is from about 10 milligram (mg) (free base weight) to about 400 mg (free base weight).

Embodiment 79. The method of any one of embodiments 1-73, wherein said effective amount of said p75^{NTR} receptor modulator or said pharmaceutically acceptable salt thereof is from about 10 milligram (mg) (free base weight) to about 300 mg (free base weight).

Embodiment 80. The method of any one of embodiments 1-73, wherein said effective amount of said p75^{NTR} receptor modulator or said pharmaceutically acceptable salt thereof is from about 10 milligram (mg) (free base weight) to about 200 mg (free base weight).

Embodiment 81. The method of any one of embodiments 1-73, wherein said effective amount of said p75^{NTR} receptor modulator or said pharmaceutically acceptable salt thereof is from about 10 milligram (mg) (free base weight) to about 100 mg (free base weight).

Embodiment 82. The method of any one of embodiments 1-81, wherein said administering comprises administering a pharmaceutical composition that comprises: said effective amount of said p75^{NTR} receptor modulator, or said pharmaceutically acceptable salt thereof; and a pharmaceutically acceptable excipient.

Embodiment 83. The method of any one of embodiments 1-82, wherein said effective amount of said p75^{NTR} receptor modulator or said pharmaceutically acceptable salt thereof is formulated in the form of a tablet.

Embodiment 84. The method of any one of embodiments 1-82, wherein said effective amount of said p75^{NTR} receptor modulator or said pharmaceutically acceptable salt thereof is formulated in a capsule.

Embodiment 85. The method of any one of embodiments 1-84, wherein said administering is performed more than once daily.

Embodiment 86. The method of any one of embodiments 1-84, wherein said administering is performed twice daily.

Embodiment 87. The method of any one of embodiments 1-84, wherein said administering is performed three times daily.

Embodiment 88. The method of any one of embodiments 1-87, wherein said administering is orally.

Embodiment 89. The method of any one of embodiments 1-88, wherein said administering comprises administering to said subject said effective amount of said p75^{NTR} receptor modulator or said pharmaceutically acceptable salt thereof for a duration of at least three months.

Embodiment 90. The method of any one of embodiments 1-88, wherein said administering comprises administering to said subject said effective amount of said p75^{NTR} receptor modulator or said pharmaceutically acceptable salt thereof for a duration of at least six months.

Embodiment 91. The method of any one of embodiments 1-90, wherein the subject exhibits or is determined to exhibit mild to moderate Alzheimer's disease according to McKhann (2011) criteria within six months prior to said administration.

Embodiment 92. The method of any one of embodiments 1-90, wherein the subject exhibits or is determined to exhibit mild to moderate Alzheimer's disease according to McKhann (2011) criteria within three months prior to said administration.

Embodiment 93. The method of any one of embodiments 1-92, wherein the subject exhibits or is determined to be at high risk or in a pre-clinical or prodromal state of Alzheimer's disease as determined by genetic risk and/or biomarkers within six months prior to said administration.

Embodiment 94. The method of any one of embodiments 1-92, wherein the subject exhibits or is determined to be at high risk or in a pre-clinical or prodromal state of Alzheimer's disease as determined by genetic risk and/or biomarkers within three months prior to said administration.

Embodiment 95. The method of any one of embodiments 1-94, wherein the subject exhibits or is determined to exhibit an ε4 allele of apolipoprotein E (ApoE) gene.

Embodiment 96. The method of any one of embodiments 1-94, wherein the subject exhibits or is determined to exhibit no ε4 allele of apolipoprotein E (ApoE) gene.

Embodiment 97. The method of any one of embodiments 1-96, wherein the subject is between the ages of 50 and 90.

Embodiment 98. The method of any one of embodiments 1-96, wherein the subject is younger than about 72 years of age.

Embodiment 99. The method of any one of embodiments 1-96, wherein the subject is of about 72 years of age or older.

### EXAMPLES

### Example 1. Drug Manufacture and Stability

The salt of Formula (II), also known as Tris[(2S,3S)-2-amino-3-methyl-N-(2-morpholinoethyl)pentanamide] tetra(monohydrogensulfate), monosulfate, a molecular formula of C₃₆H₈₅N₉S₅O₂₀, and a molecular weight of 1220.43 gm/mole. This compound is a white solid and has aqueous solubility of greater than 30 mg/mL and very good stability under accelerated temperature and humidity conditions. GMP lots have no impurities above the LOD following storage at ICH stress conditions, indicating sufficient stability of the drug substance. Preparation of the clinical dosage form consists of a dry fill of the drug substance into gelatin capsules with no excipients. For the Phase II study, capsules with the salt of Formula (II)(200 mg or 400 mg, free base weight) are machine-filled and packaged in blister strips with aluminum foil backing, which are stored in cardboard boxes. Matching placebo capsules are also available in the same packaging.

Data show no significant changes in the drug substance through 18 months at −20° C., 5° C., 25° C./60% RH or 40° C./75% RH other than moisture content, which decreases under all conditions monitored, particularly at the refrigerated (5° C.) and frozen (−20° C.) conditions.

Similarly, data for a drug substance lot for the Phase I trial showed no significant changes related to assay or impurities over a six-month time frame under accelerated conditions, or at 48 months when stored at 25° C./60% RH (the recommended storage condition).

A stability study of capsules filled with 200 mg of the salt of Formula (II) (free base weight) packed in PVC-PE-PVdC/Alu at ambient conditions (15-25° C./20-60% RH) shows good drug stability. The provisional shelf-life is set at 18 months, stored in PVC-PE-PVdC/Alu blister strips at ambient conditions.

### Example 2. Regional Brain Glucose Metabolism (18F-FDG-PET)

The specific ¹⁸F FDG-PET scan for early detection of AD is conducted at the first and final visits. Quantitative and qualitative estimates of the cerebral glucose rate may be done by using FDG-PET as evident metabolic reduction is already present in patients at early stages of Alzheimer's disease. ¹⁸F FDG-PET scanning is an imaging biomarker for synaptic function. Analyses include anatomical, statistical or voxel-based regions of interest. Patients receiving daily treatment with a pharmaceutical composition comprising the salt of Formula (II) show a reduction in the extent of ongoing decrease in cerebral glucose rate following administration of the composition over a course of time compared to groups of subjects receiving placebo or no treatment. At baseline and final Visit the specific 18F FDG-PET scan for early detection of AD (Mosconi et al., 2010) was conducted. Quantitative and qualitative estimates of the cerebral glucose rate may be done by using FDG-PET as evident metabolic reduction was already present in patients at early stages of Alzheimer's disease (Berti et al., 2010; Minoshima et al., 1997).

### Example 3. Brain MRI Regions of Interest Volumes (ROI) and Voxel Based Whole Brain Analyses

Volumetric MRI is conduction at the first and final visits. Volumetric analyses of the whole brain, hippocampus and other regions of interest are conducted. Measures of regional volumes as determined by MRI are regarded as standard measures of brain degeneration that are known to occur in AD. Volumes of the lateral ventricles are also assessed. Increasing ventricular volume is a marker for a diffuse increase in brain atrophy. Patients receiving daily treatment with a pharmaceutical composition comprising the salt of Formula (II) show a reduction in the extent of ongoing decrease in one or more regional volumes during the treatment period compared to groups of subjects receiving placebo or no treatment. They also demonstrate a reduction in the extent of ongoing increase in ventricular volume during the treatment. Longitudinal AD studies demonstrate atrophy of selected brain regions over 6-month intervals as well as increased volume of lateral ventricles, each indicative of neuronal degeneration. The following Regions of interest were investigated in the ITT population: MRI Volume of the lateral ventricles in mm3—longitudinal stream change, MRI Average volume of the hippocampus in mm3—longitudinal stream change, MRI Average Volume of the entorhinal cortex in mm3—longitudinal stream change, MRI Total brain volume in mm3 change. Baseline and final Visit MRI scan were used to measure interval changes in volumes. Various cortical regions such as the Cingulate cortex were also assessed. Volumetric MRI was also conducted using a whole brain voxel-based approach in which brain regions are identified that demonstrated volume loss during the treatment period with the extent of volume loss statistically greater in the placebo group compared to the drug treated group. The identified brain regions are similar in distribution to those having been previously identified to be particularly vulnerable to degeneration in AD. Thus the drug is demonstrated to slow volume loss in brain regions known to be particularly vulnerable to degeneration in AD.

### Example 4. CSF Biomarkers

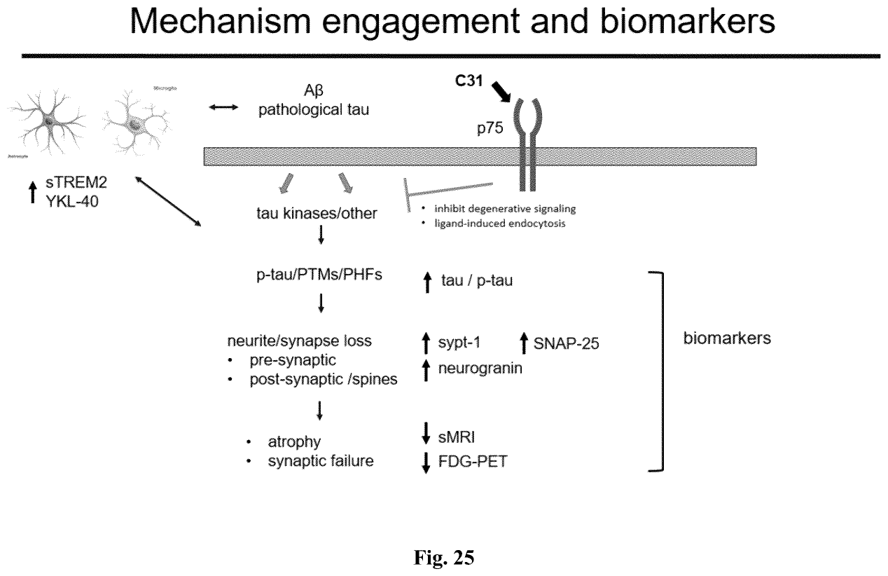

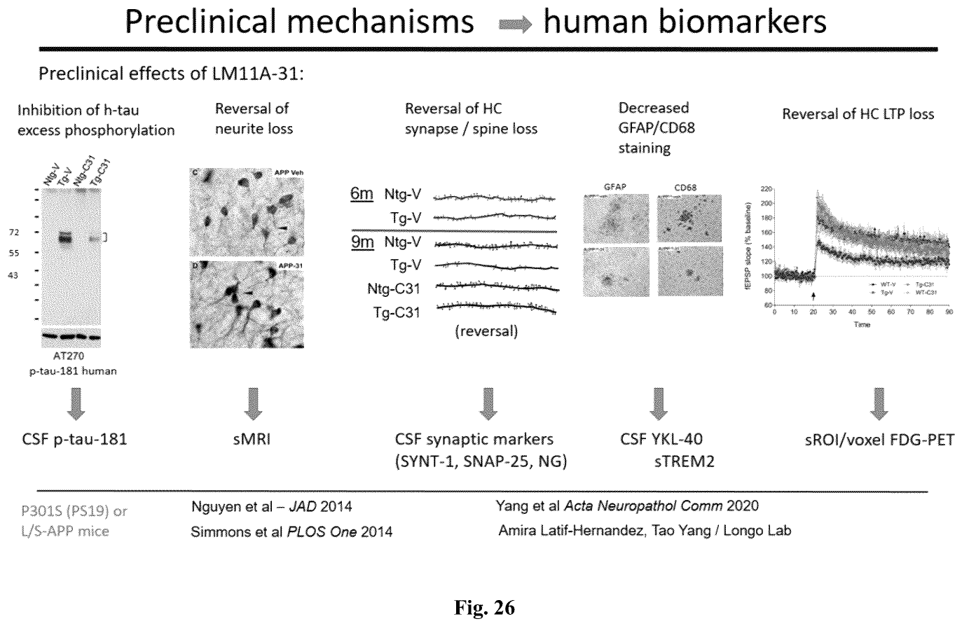

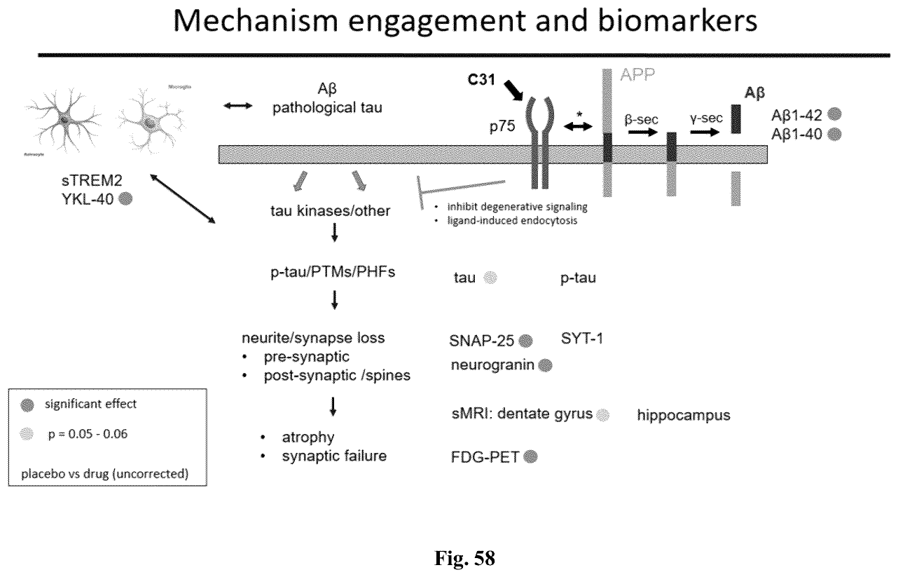

CSF samples were obtained at screening Visit and final Visit via lumbar puncture to determine levels of tau, p-tau, Aβ40, Aβ42 and AChE activity as an initial set of CSF biomarkers. Aliquots of CSF were made available for emerging CSF biomarker methods such as measurement of additional biomarkers including neurofilaments light chain, neurogranin, SNAP-25, synaptotagmin, YKL-40, TREM2, tau oligomers and for measurement of drug level in selected patients based on the time period between last dose of drug and CSF sampling (FIG. 25 and FIG. 58).

Baseline CSF samples are obtained prior to administration via lumbar puncture. These samples are used to determine levels of tau, p-tau, Aβ40, Aβ42, AChE, neurofilament light chain, SNAP-25, synaptotagmin-1, neurogranin, sTREM, and YKL-40. Aliquots of CSF are also made available for emerging CSF biomarker methods such as measurement of tau oligomers and for measurement of drug level in selected patients based on time period between last dose of the salt of Formula (II) and CSF sampling. Daily treatment with a salt of Formula (II) results in a reduction in the degree of expected increase in levels of biomarkers indicating ongoing degeneration, inflammation or gliosis such as tau, p-tau, Aβ40, Aβ42, neurofilament light chain, SNAP-25, synaptotagmin-1, neurogranin, sTREM, and YKL-40 in the CSF compared to the changes in levels found in placebo or untreated subjects. Many of these biomarkers can also be measured in blood or plasma. The change in values over the time period from pre-treatment to post-treatment are of particular importance. The drug demonstrates the ability to slow progression of increase of biomarkers indicating neuronal degeneration, gliosis and neuro-inflammation. It also demonstrated the ability to lower levels of Aβ42 and 40 consistent with the known ability of the p75 receptor to regulate Aβ production. Absolute levels at single time points of neurofilament light chain, SNAP-25, synaptotagmin-1, neurogranin, sTREM, and YKL-40 in CSF or blood or plasma are of less value given differences in assay techniques and wide variation in absolute levels within normal subjects and within and across AD subjects. In contrast, absolute levels of Aβ42 and 40 at baseline are useful biomarkers indicating the presence of amyloid pathology as low levels of Aβ42 and 40 at a given time point are consistent with amyloid pathology.

### Example 5. Cognitive and Clinical Function Endpoints

Exploratory outcome evaluations involved the assessment of cognitive and clinical function (Table 1). The ADAS-cog and MMSE testing demonstrated significant decline of cognitive function in the placebo group. Directionality of slowing progression of worsening in the ADAS-cog and MMSE indicated a favorable effect of drug treatment. The NTB failed to detect worsening in the placebo group. Items from the Neurological Testing Battery (NTB), together with the ADAS-Cog 13, Spatial Navigation Test (AMUNET) were performed at Baseline, Week 12 and Week 26 (Visits 2, 4 and 5). While the MMSE was used primarily as a screening and baseline disease staging tool, its repeated administration at Week 26 provided an additional exploratory cognitive outcome measure. The Geriatric Depression Scale (GDS) was assessed at each Visit (Screening, Baseline, Week 4, 12, 26: Visits 1-5) and the Clinical Global Impression test (CGI) with Severity was assessed at Screening (Visit 1) and then the Improvement was subsequently assessed at Weeks 12 and 26 (Visits 4 and 5). For each test, whether categorical or numeric, all raw scores were considered interval in nature. The change from Screening (Week −8) or Baseline (Week 0) was therefore be derived for each patient. Actual values at each Visit and Changes from Screening/Baseline were summarized.

TABLE 1
Cognitive and Clinical Function Endpoints

| | | |
|---|---|---|
| Outcome | Score Range (units) | Scale Direction |

Neurological Test Battery (NTB)

| | | |
|---|---|---|
| DST | 0-24 | High score (24 = Best) |
| | (Count correct | |
| | sequences) | |
| CFT | 0-Unlimited | High score |
| | (Count acceptable | |
| | words) | |
| COWAT | Letter C = Accept* | High score |
| | Letter F = Accept* | |
| | Letter L = Accept* | |
| | Total = C + F + L | |
| | (Count correct words) | |
| DSST | 0-117 | High score (117 = Best) |
| | (Count correct | |
| | substitutions) | |

Other Cognitive Function Tests

| | | |
|---|---|---|
| AMUNET | Navigation | AMUNET analysis report |
| | AlloEgo/Ego/Allo/ | |
| | Delayed | |
| ADAS-cog | 0-85 | Low score best |
| (13 items) | (Count incorrect items) | (0 = All correct) |

Clinical Function Tests

| | | |
|---|---|---|
| GDS | 0-15 | Low score best |
| | (Score) | (0-4 = Normal/ |
| | count/classification) | 12-15 Severe depression) |
| CGI - Severity | 1-7 | Low score best |
| (At Baseline) | (Classification) | (1 = Normal/7 = Extremely Ill) |
| CGI - | 1-7 | Low score best |
| Improvement | (Classification) | (1-Very much improved/ |
| | | 7 = Very much worse) |

Disease Staging

| | | |
|---|---|---|
| MMSE | 0-30 | High score best |
| | (Count correct items) | (0 = Severe impairment/ |
| | | 30 = No impairment) |

*Footnote 1: Accept = Number of acceptable words beginning with ‘C, F and L’ and recorded within 60 seconds

### Example 6. Alzheimer's Disease Assessment Scale—13 Items (ADAS-Cog 13 Items)

Alzheimer's Disease Assessment Scale—13 items (total score=85) is used as a parameter for exploratory outcome analysis. ADAS-cog is a psychometric instrument designed to evaluate the severity of cognitive and non-cognitive behavioral dysfunctions characteristic of people with AD. The cognitive portion assesses memory, language and praxis functions. Most items are rated on a scale of 0 to 5, where 0 indicates no impairment and 1 to 5 indicates very mild (1), mild (2), moderate (3), moderately severe (4) or severe impairment (5) respectively. Other items are rated on the presence or absence of a characteristic number of errors or severity of errors. The total scores from the ADAS-cog sub-scales range from 0 (no impairment) to 85 (errors in all sub-tests).

A positive (i.e. increasing) change of the score indicates cognitive worsening. In a meta-analysis model of 52 Alzheimer's Disease trials involving 19,972 mild-moderate disease subjects, a spontaneous decline in performance corresponding to an increase in ADAS-cog score of 5.5 points over one year, pointing to an expected average increase in score of 2.75 points over a 6-month period.

ADAS-Cog was assessed at Visits 2, 4 and 5, as well as at the early discontinuation Visit, if applicable.

Rater: Neurologist, psychologist or other personal (listed as either Principal Investigator or Sub-Investigator) with documented training in psychometric rating.

While the MMSE was used primarily as a screening and baseline disease-staging tool, its administration at Week 26 provided an additional exploratory cognitive outcome measure. Longitudinal evaluations of MMSE performance in Alzheimer's disease subjects demonstrate annual rates of decrease ranging from 2.8 to 3.4 points, pointing to an expected decline of approximately 1.6 points over a 6-month period. Daily treatment with a salt of Compound (II) results in a reduction in the extent of expected decline in MMSE scores compared to the decline in scores occurring in subjects not treated or treated with placebo.

Daily treatment with a salt of Compound (II) results in a reduction in the extent of expected decline in ADAS-Cog scores relative to previous visits or a baseline value prior to administration. For example, over the course of treatment of 6 months, subjects receiving daily treatment with a salt of Compound (II) may show a reduction in the extent of declining performance in ADAS-Cog scores, or may show a reduction in the extent of declining performance in ADAS-Cog scores by 1, 2, 3, 4, 5, 6, 7, 8, 9, 10, 15, 20, 25, 30, 35, 40, 50, 60, 70 or 85 points. Subjects may show an ADAS-Cog increase of score (decrease in performance) of less than an increase in score observed in a six-month span prior to administration indicating a slowing in the rate of loss of performance. For example, a subject may have an increase in ADAS-Cog of 6 points (decline in performance) in a six month span prior to administration and an increase in ADAS-Cog of less than 6 points, such as an increase in 4, 3, 2 or 1 points in a six month span (indicating a slowing of cognitive decline) that includes treatment with a salt of Formula (II). Daily treatment with a salt of Compound (II) results in a reduction in the extent of expected decline in ADAS-cog performance (increased score) compared to the decline in performance occurring in subjects not treated or treated with placebo.

ADAS-cog. In the placebo group, ADAS-cog-13 score increased by 2.44 points. Using a linear model, this change of over the 26-week period would project to 4.88 at one year and 7.32 at 18 months, a range of cognitive decline as measured by ADAS-cog-13 in mild-moderate AD expected from prior reports. In the drug group (intention to treat data, ITT), the progression compared to placebo in the LS mean ADAS-cog-13 score increased by 7.1% in the 200 mg group and decreased by 35.3% in the 400 mg group over the 26-week treatment period. For ADAS-cog-11, the progression compared to placebo of the LS mean score decreased by 9.8% in the 200 mg group and decreased by 42.7% in the 400 mg group. For ADAS-cog-6 score, progression increased by 7.1% in the 200 mg group and decreased by 31.4% in the 400 mg group. Consistent with the low clinical assessment power of the study with the relatively small treatment groups in the context of clinical measures of approximately 80 subject each, and assessment of a 6-month rather than 18-month time point, measures of slowing of progression demonstrated notable trends in a favorable direction but were not statistically significant.

### Example 7. Mini Mental Status Exam (MMSE)

Mini Mental Status Exam (MMSE). The MMSE can be used by a physician to evaluate a patient's condition over the course of administration. A baseline value prior to administration of a salt of Formula (II) can serve as a basis for baseline disease stage and also for the assessment of the effect of treatment on slowing loss of cognitive function. The expected decline in MMSE over a six month period generally ranges 1-3 points out of the total of 30 points possible in the MMSE test.

Subjects may show a decrease in the extent of expected decline in MMSE over a six month period following or in association with treatment with a salt of Compound (II).

In the placebo group, the MMSE score (LS mean, ITT) decreased by 1.65 points. Using a linear model, this progression over the 26-week period would project to 3.3 points at one year and 4.95 at 18 months, a range of cognitive decline in mild-moderate AD expected from prior reports. In the drug group, degree of progression relative to placebo was decreased by 15% in the 200 mg group and by 30.2% in the 400 mg group. Consistent with the low clinical assessment power of the study with the relatively small treatment groups in the context of clinical measures of approximately 80 subject each, and assessment of a 6-month rather than 18-month time point, measures of slowing of progression demonstrated notable trends in a favorable direction but were not statistically significant.

### Example 8. Clinical Global Impression Scale

The Clinical Global Impressions Scale—Severity (CGI-S) and—Improvement (CGI-I) can be used by a physician to evaluate a patient's condition over the course of administration. A baseline value prior to administration of a salt of Formula (II) can serve as a basis for the assessment. The subjective categorical values of the CGI-I are as follows: 1=very much improved since the initiation of treatment; 2=much improved; 3=minimally improved; 4=no change from baseline; 5=minimally worse; 6=much worse; and 7=very much worse since the initiation of treatment. The CGI-S was assessed at Visit 1, and CGI-I for evaluation of improvement of subject's condition at Visit 4, 5 and early discontinuation Visit, if applicable. Rater: Neurologist, psychologist or other personal (listed as either Principal Investigator or Sub-Investigator) with documented training in psychometric rating.

Daily treatment with a salt of Formula (II) results in a mitigation of worsening of scores (lower scores) in Clinical Global Impression scores occurring during the treatment period compared to a baseline value.

### Example 9. Neurological Testing Battery (NTB)

Daily treatment with a salt of Formula (II) results in a decreased in the degree of decline of at least one element of the NTB compared to a pretreatment baseline measurement. The NTB comprises tests of digit span, category fluency, controlled oral word association and digit symbol substitution. These tests are performed approximately three months prior to treatment, after 10-14 weeks of treatment and after completion of treatment.

### Digit Span

Study participants were read sequences of numbers and required to repeat them as heard in the first phase of the test (Digits Forward). In the second phase of the test, Digits Backwards, study participants were required to repeat the sequence in the reverse order. Two trials were administered for each sequence length and 1-point was awarded for each sequence correctly repeated. Testing was performed at Visits 2, 4 and 5, as well as at the early discontinuation Visit, if applicable. The rater was a trained psychologist or physician

### Category Fluency Test

In this test, study participants were required to generate words from a specific category (usually animals) in one minute. Performance across the minute was scored according to acceptable rules to yield the total number of correct responses. This test measures working memory and other aspects of executive function, including planning, strategy and aspects of language and especially fluency. Testing was performed at Visits 2, 4 and 5, as well as at the early discontinuation Visit, if applicable. The rater was a trained psychologist or physician.

### Controlled Oral Word Association Test (COWAT)

The COWAT measures a person's ability to make verbal associations to specified letters (i.e., C, F, and L), evaluates the spontaneous production of words beginning with a given letter and is able to detect changes in word association fluency often found with various disorders. COWAT testing was performed at Visits 2, 4 and 5, as well as at the early discontinuation Visit, if applicable. The rater was a trained psychologist or physician.

### Digit Symbol Substitution Test

In the Digit Symbol Substitution Test (DSST), the patient was required to match symbols with their corresponding digit. The test consists of 9 digit symbols, which had to be matched with their corresponding numerical digit. The patients had limited amount of time to enter the correct symbol for each digit. DSST was performed at Visits 2, 4 and 5, as well as at the early discontinuation Visit, if applicable. The rater was a trained psychologist or physician.

### Example 10. Spatial Navigation with AMUNET

Impaired orientation in space is a frequently reported symptom in AD patients. Spatial navigation impairment occurs early in the development of AD and can be used for monitoring of the disease progression or for evaluation of presymptomatic AD.

The two modes of spatial navigation include egocentric and allocentric navigation. Egocentric navigation uses information about distances and angles from the subject positions processing proprioreceptive information, whilst allocentric navigation is hippocampus dependent and uses a flexible representation of a distal landmarks ensemble independent of actual subject positions. The parietal cortex including precuneus, and especially the hippocampus, is involved in spatial navigation performance. Impairments are particularly found in patients suffering from memory deficits related to the hippocampal area—correlating to prodromal AD pathologic findings, presumably a signal for preclinical AD.

Memory paradigms used with human study participants suffering from AD typically feature tests of episodic verbal memory, paired associative learning or visual recognition memory. These tasks are very different to the memory paradigms in rodents, where the Morris water maze (MWM) is employed in preclinical studies for the development of new medicinal products for AD.

AMUNET, the computer-based simulation of MWM, tests the two basic types of navigation: world-centered—allocentric (hippocampus-dependent) and body-centered—egocentric (parietal cortex-dependent). Both of these paradigms are controlled by the structures involved in early Alzheimer's disease pathology. Allocentric navigation is independent of an individual's position and distal cues are used for navigation, while egocentric navigation depends on an individual's position and the start position is used for navigation. AMUNET computer simulation is a map view of the arena projected on computer screen where the participant uses a touch screen to identify the target position. The arena in the computerized version of the MWM was shown as a large white circle with the start position (medium-sized red circle) and 2 orientation cues (yellow and green lines) on its perimeter. A small red circle inside the arena represents the goal.

AMUNET was conducted at Visits 2, 4 and 5, as well as at the early discontinuation Visit, if applicable. The rater was a trained psychologist or physician. The Results from the AMUNET testing after week 12 and week 26 were compared to the baseline tests. Four different result-subsets were compared for ITT and PP population as well as two subsets of subjects within the groups showing mild or moderate symptoms of AD. The four result subsets tested consisted of:
AlloEgoNavigation: The capability of spatial navigation both with the aid of visible orientation cues that are not placed near the goal (allocentricnavigation) and with the aid of information about the direction and the distance from the start that is needed to find the goal(egocentricnavigation).
EgoNavigation: Focused on egocentric navigation.
AlloNavigation: Focused on allocentric navigation
DelayedNavigation: Focused on delayed allocentric navigation.

Daily treatment with a composition comprising a salt of Formula (II) results in a decrease in the decline in performance in testing of either allocentric or egocentric spatial navigation with Amunet as compared to a baseline assessment prior to administration of the composition. Amunet is conducted approximately three months prior to administration of the composition, after 10-14 weeks of treatment and after completion of treatment.

### Example 11: Geriatric Depression Scale (GDS)

The Geriatric Depression Scale (GDS) is a useful validated screening tool to facilitate assessment of depression in older adults especially when baseline measurements are compared to subsequent scores. The GDS Short Form consists of 15 Items and takes about 5 to 7 minutes to be completed. Of the 15 items 10 indicate the presence of depression when answered positively, while the rest indicate depression when answered negatively. A score of 0 to 4 is normal, depending on age, education, and complaints; a score of 5 to 8 indicates a mild depression, 9 to 11 reflects a moderate depression, 12-15 a severe depression. Evaluation of the GDS was performed at screening and only patients with GDS score <5 were enrolled in the study.

The GDS was assessed at Visits 1, 2, 3, 4, 5 and early discontinuation Visit, if applicable. The rater was a trained psychologist or physician.

### Example 12. Phase I, Randomized, Double-Blind, Placebo Controlled Single- and Multiple Ascending Dose Study to Evaluate the Safety, Tolerability, and Pharmacokinetics of the Salt of Formula (II) in Healthy Elderly Subjects

The randomized, double-blind, placebo-controlled study was designed to test safety and tolerability of a 600 mg twice a day of LM11A-31-BHS (the salt of Formula (II)) administered orally to healthy elderly volunteers for a period of 10 days. The p75 receptor had never been specifically targeted in a human trial, hence the critical important of determining of targeting this receptor is safe. Given the fundamental and complex roles of the p75 receptor, it was not known if targeting or modulating this receptor would be tolerable.

Trial specific stopping rules were set to protect the trial subjects. Dosing would have been stopped for all subjects and the trial terminated if any of the stopping rules had occurred.

15 healthy adults were screened within three weeks prior to the baseline visit. Following 5 screening failures, 10 healthy adults, 5 aged 18-64 years and 5 aged 65-84 years were enrolled. Participants were split into two reporting groups, an experimental group receiving twice daily oral administration of 600 mg (1200 mg total daily) LM11A-31-BHS and a placebo group receiving twice daily oral administration of a 600 mg placebo. Characterization of each arm of the study is shown in Table 2 and characterization of each reporting group is shown in Table 3.

TABLE 2
Description of each arm in the phase I study
Period 1

| | |
|---|---|
| Period 1 title | Overall trial (overall period) |
| Is this the baseline period? | Yes |
| Allocation method | Randomised - controlled |
| Blinding used | Double blind |
| Roles blinded | Subject, Investigator, Monitor |

Arms

| | |
|---|---|
| Are arms mutually exclusive? | Yes |
| Arm title | LM11A-31-BHS |

Arm description:
600 mg LM11A-31_BHS in the morning and in the evening
(total daily dosage = 1200 mg)

| | |
|---|---|
| Arm type | Experimental |
| Investigational medicinal product | LM11A-31-BHS |
| Investigational medicinal product | LM11A-31-BHS |
| Other name | |
| Pharmaceutical forms | Capsule, hard |
| Routes of administration | Oral use |

Dosage and administration details:
3 capsules with 200 mg LM11A-31-BHS administered b.i.d. with
approximately 240 mL of water

| | |
|---|---|
| Arm title | Placebo |

Arm description:
600 mg placebo in the morning and in the evening
(total daily dosage = 1200 mg)

| | |
|---|---|
| Arm type | Placebo |
| Investigational medicinal product | Placebo |
| Investigational medicinal product | |
| Other name | |
| Pharmaceutical forms | Capsule, hard |
| Routes of administration | Oral use |

Dosage and administration details:
3 capsules with 200 mg placebo administered b.i.d. with approximately
240 mL of water
Period 1

TABLE 3
Group characteristics for subjects in each arm of the phase I study
Reporting groups

| | |
|---|---|
| Reporting group title | LM11A-31-BHS |

Reporting group description:
600 mg LM11A-31_BHS in the morning and in the evening
(total daily dosage = 1200 mg)

| | |
|---|---|
| Reporting group title | Placebo |

Reporting group description:
600 mg placebo in the morning and in the evening
(total daily dosage = 1200 mg)

| | LM11A-31- | | |
|---|---|---|---|
| Reporting group values | [symbol] | Placebo | Total |
| Number of subjects | 8 | 2 | 10 |
| Age categorical | | | |
| Units [symbol] Subjects | | | |
| Adults (18-64 years) | 4 | 1 | 5 |
| From 65-84 years | 4 | 1 | 5 |
| Gender categorical | | | |
| Units [symbol] Subjects | | | |
| Female | 4 | 1 | 5 |
| Male | 4 | 1 | 5 |

[symbol]

indicates data missing or illegible when filed

End point values recorded included any adverse events, systemic pharmacokinetics of LM11A-31-BHS, and cerebrospinal fluid (CSF) levels of LM11A-31-BHS of orally adminstered LM11A-31-BHS of 600 mg twice daily doses. The results of measuring these end points in the study participants are summarized in Tables 4-9.

TABLE 4
Description of end points reporting groups
End points reporting groups

| Reporting group title | LM11A-31-BHS |
|---|---|
| Reporting group description: | |
| 600 mg LM11A-31_BHS in the morning and in the evening | |
| (total daily dosage = 1200 mg) | |
| Reporting group title | Placebo |

Reporting group description:
600 mg placebo in the morning and in the evening
(total daily dosage = 1200 mg)

TABLE 5
Summary of adverse effects observed in participants of phase I trial
Primary: To investigate the safety and tolerability of 600 mg b.i.d.
of LM11A-31-BHS administered to healthy elderly volunteers for
a period of 10 days in comparison to placebo

| | |
|---|---|
| End point title | To investigate the safety and tolerability of 600 mg |
| | b.i.d. of LM11A-31-BHS administered to healthy |
| | elderly volunteers for a period of 10 days in comparison |
| | to placebo |

End point description:

| | |
|---|---|
| End point type | Primary |

End point timeframe:
continuouse until day 11, last dose is given in the
morning of day

### 10 Notes

[1]—No statistical analyses have been specified for this primary end point. It is expected there is at least one statistical analysis for each primary end point.

Justification: Because of the nature of the study, the results were only descriptively summerized.

| | LM11A-31- | |
|---|---|---|
| End point values | [symbol] | Placebo |
| Subject group type | Reporting | Reporting |
| Number of subjects analysed | 8 | 2 |
| Units: drug related adverse events | | |
| mild drug related adverse events | 4 | 0 |
| moderate drug related adverse events | 0 | 0 |
| severe drug related adverse events | 0 | 0 |
| drug related serious adverse events | 0 | 0 |

[symbol]

indicates data missing or illegible when filed

TABLE 6
Summary of systemic pharmacokinetics of LM11A-31-BHS
Secondary: To investigate the systemic pharmacokinetics of 600 mg
b.i.d. doses of LM11A-31-BHS administered orally for a period of
10 days in healthy elderly volunteers

| | |
|---|---|
| End point title | To investigate the systemic pharmacokinetics of 600 mg |
| | b.i.d. doses of LM11A-31-BHS administered orally for |
| | a period of 10 days in healthy elderly volunteers |

End point description:

| | |
|---|---|
| End point type | Secondary |

End point timeframe:
day 1, 2, 5, 8, 10 and 11 with several timepoints on day 1 and 10

| | LM11A-31- | |
|---|---|---|
| End point values | [symbol] | Placebo |
| Subject group type | Reporting | Reporting |
| Number of subjects analysed | 8 | 2 |
| Units: Cmax [ng/ml] | | |
| arithmetic mean (standard deviation) | | |
| day 1 | 1271.61 | 0 (±0) |
| | (±606.76) | |
| day 10 | 885.18 | 0 (±0) |
| | (±522.75) | |

[symbol]

indicates data missing or illegible when filed

TABLE 7
Summary of levels of LM11A-31-BHS in CSF of participants in
phase I trial

| Secondary: To evaluate cerebrospinal fluid (CSF) levels of | |
|---|---|
| LM11A-31-BHS under the conditions of this trial | |
| End point title | To evaluate cerebrospinal fluid (CSF) levels of |
| | LM11A-31-BHS under the conditions of this trial |
| End point description: | |
| End point type | Secondary |

End point timeframe:
several timepoints on day 10

| | LM11A-31- | |
|---|---|---|
| End point values | [symbol] | Placebo |
| Subject group type | Reporting | Reporting |
| Number of subjects analysed | 8 | 2 |
| Units: Cmax [ng/ml] | | |
| arithmetic mean (standard deviation) | 56.00 | 0 (±0) |
| | (±24 [symbol] 54) | |

[symbol]

indicates data missing or illegible when filed

TABLE 8
Breakdown of serious adverse effects observed in phase I trial

| | LM11A-31- | |
|---|---|---|
| Serious adverse events | [symbol] | Placebo |
| Total subiects affected bv serious | | |
| adverse events | | |
| subjects affected/exposed | 1/8 (12.50%) | 0/2 (0.00%) |
| number of deaths (all causes) | 0 | 0 |
| number of deaths resulting | 0 | 0 |
| from adverse events | | |
| Nervous system | | |
| disorders Headache | | |
| subjects affected/exposed | 1/8 (12.50%) | 0/2 (0.00%) |
| occurrences causally related | 0/1 | 0/0 |
| to treatment/all | | |

[symbol]

indicates data missing or illegible when filed

TABLE 9
Breakdown of non-serious adverse effects observed in phase II trial

| | LM11A-31- | |
|---|---|---|
| Non-serious adverse events | BHS | Placebo |
| Total subjects affected by | | |
| non-serious adverse events | | |
| subjects affected/exposed | 7/8 (87.50%) | 2/2 (100.00%) |
| Investigations | | |
| Gamma-glutamyltransferase | | |
| increased | | |
| subjects affected/ | 1/8 (12.50%) | 0/2 (0.00%) |
| exposed occurrences (all) | 1 | 0 |
| Respiratory, thoracic and | | |
| mediastinal disorders | | |
| Oropharyngeal pain | | |
| subjects affected/ | 0/8 (0.00%) | 2/2 (100.00%) |
| exposed occurrences (all) | 0 | 3 |
| Nervous system | | |
| disorders Headache | | |
| subjects affected/ | 5/8 (62.50%) | 0/2 (0.00%) |
| exposed occurrences (all) | 7 | 0 |
| Presyncope | | |
| subjects affected/ | 0/8 (0.00%) | 1/2 (50.00%) |
| exposed occurrences (all) | 0 | 1 |
| Eye | | |
| disorders | | |
| Dry eye | 0/8 (0.00%) | 1/2 (50.00%) |
| subjects affected/ | 0 | 1 |
| exposed occurrences (all) | | |
| Gastrointestinal | | |
| disorders Flatulence | | |
| subjects affected/ | 3/8 (37.50%) | 0/2 (0.00%) |
| exposed occurrences (all) | 3 | 0 |
| Diarrhea | | |
| subjects affected/ | 1/8 (12.50%) | 0/2 (0.00%) |
| exposed occurrences (all) | 1 | 0 |
| Abdominal pain | | |
| subjects affected/ | 1/8 (12.50%) | 0/2 (0.00%) |
| exposed occurrences (all) | 1 | 0 |
| Renal and urinary | | |
| disorders Polyuria | | |
| subjects affected/ | 1/8 (12.50%) | 0/2 (0.00%) |
| exposed occurrences (all) | 1 | 0 |
| Musculoskeletal and connective | | |
| tissue disorders | | |
| Arthralgia | | |
| subjects affected/ | 1/8 (12.50%) | 0/2 (0.00%) |
| exposed occurrences (all) | 3 | 0 |

### Example 13. Phase IIa, Prospective, Multi-Center, Double-Blind, Placebo-Controlled, Randomized, Adaptive-Trial-Design Study to Evaluate Safety, Tolerability, Pharmacokinetics, and Exploratory Endpoints of Either Placebo or Two Different Oral Doses of the Salt of Formula (II) in Patients with Mild to Moderate Probable Alzheimer's Disease

### Overall Study Design and Plan Description

The study was a phase IIa, multi-center, randomized, placebo-controlled, parallel group clinical study with a 26-week double-blind treatment duration and three treatment arms with two consisting of two doses of LM11A-31 (the salt of Formula (II)) (200 mg bid and 400 mg bid of free base—and one comprising placebo bid).

The primary goal of the phase IIa clinical trial was the evaluation of safety and tolerability of two doses of LM11A-31-BHS administered orally twice daily for 26 weeks versus matched placebo. Safety monitoring included the full extent of phase 2 clinical and laboratory testing. The p75 receptor had never been specifically targeted in a human trial other than the above phase 1 10d trial, hence the critical important of determining of targeting this receptor beyond 10 days is safe. Given the fundamental and complex roles of the p75 receptor, it was not known if targeting or modulating this receptor for 26 weeks would be tolerable.

### Treatments Administered

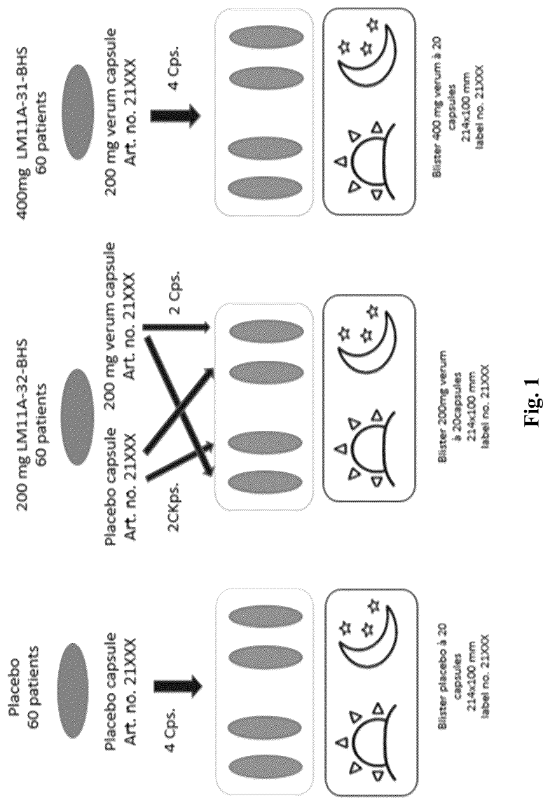

LM11A-31-BHS or the matching placebo were administered twice daily as 2 capsules of identical appearance in a blinded manner (FIG. 1).

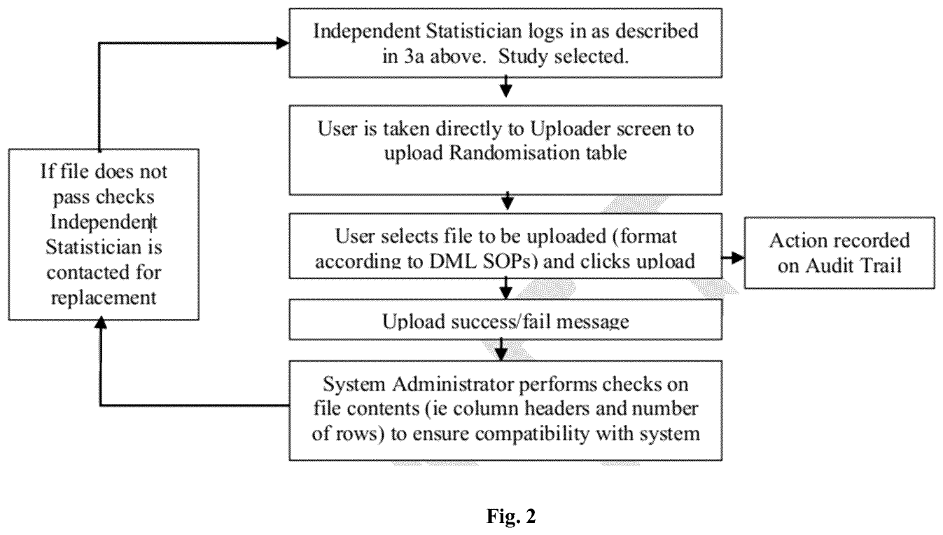

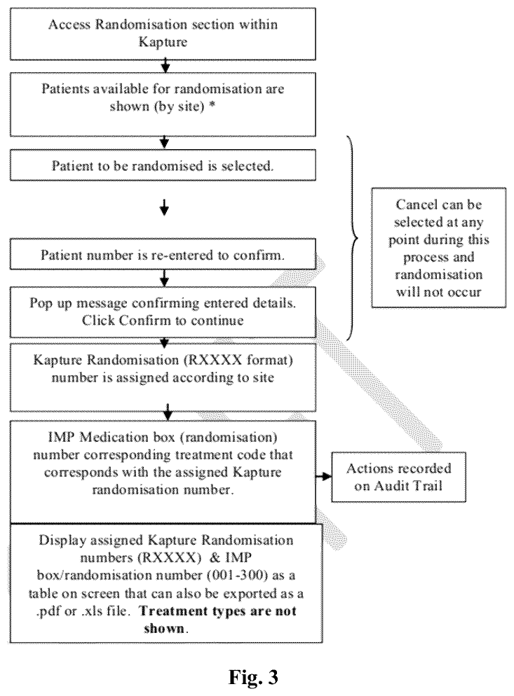

On the first treatment day, the patients were randomized to either placebo (n=80) or 200 mg bid (n=80) or 400 mg bid (n=80) LM11A-31-BHS following the procedure of FIGS. 2 and 3.

### Drug Concentration Measurements

Plasma samples were collected from all subjects who received study medication to investigate the plasma concentration of LM11A-31 and its aminoethyl morpholine metabolite. The time between PK sampling and last intake of study drug was recorded. All available samples including samples from the placebo group were analyzed. Levels of LM11A-31 and its aminoethyl morpholine metabolite were measured in selected CSF samples that were otherwise obtained for studies of CSF biomarkers.

CSF was collected from all subjects who received study medication. However, levels of LM11A-31 and its aminoethyl morpholine metabolite were measured only in CSF samples from subjects who had the last dose of study drug on the day of the CSF collection and who were based on the plasma PK results available at Quintain one of the verum arms. In addition CSF samples of 3 patients from the placebo group were analyzed as QC check.

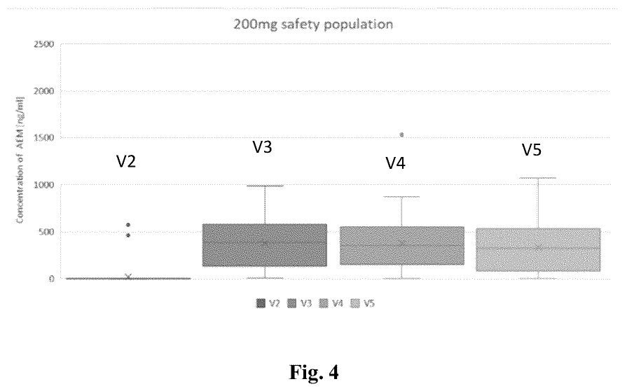

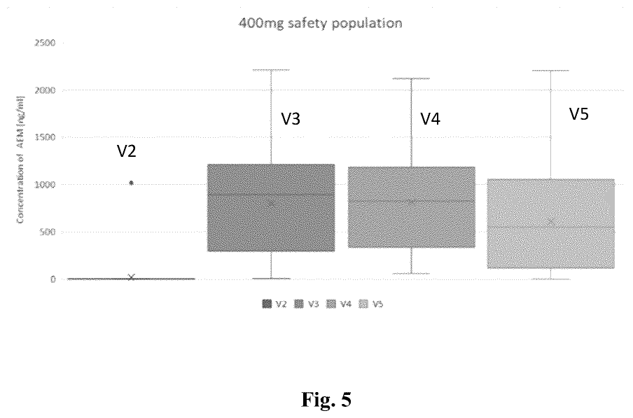

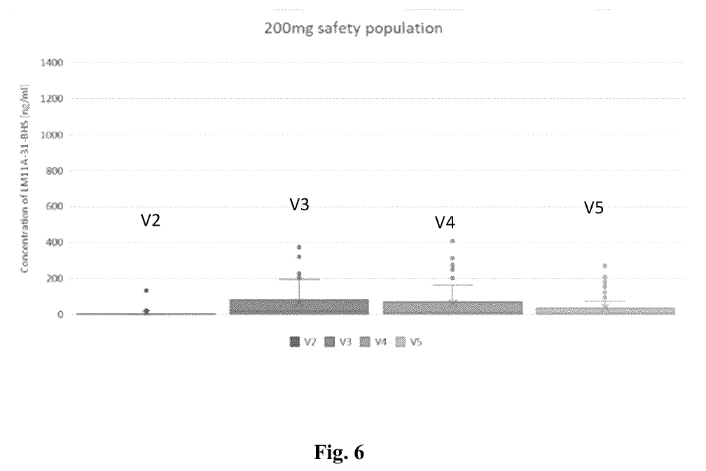

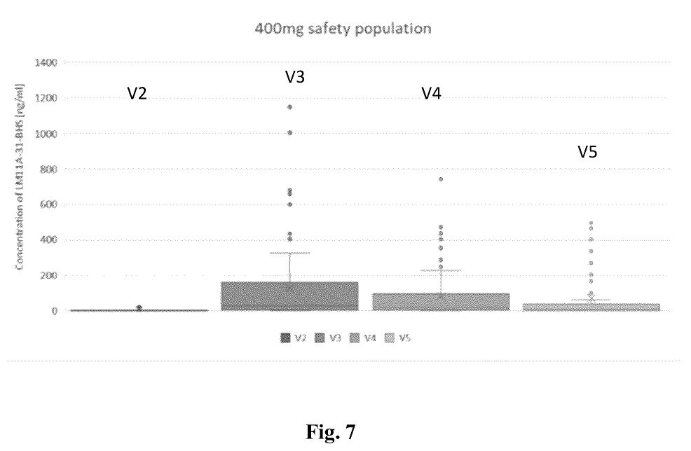

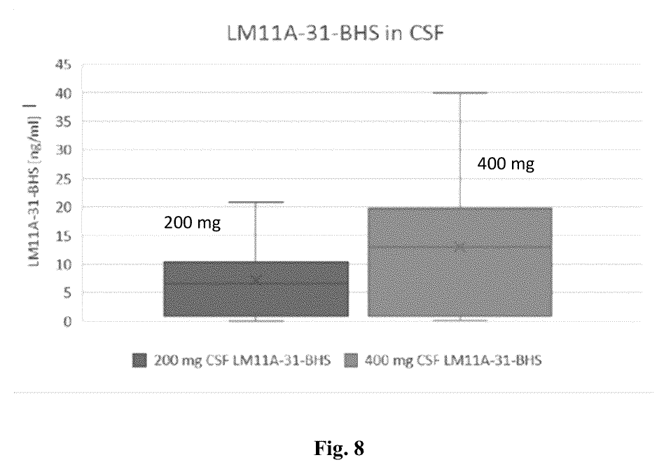

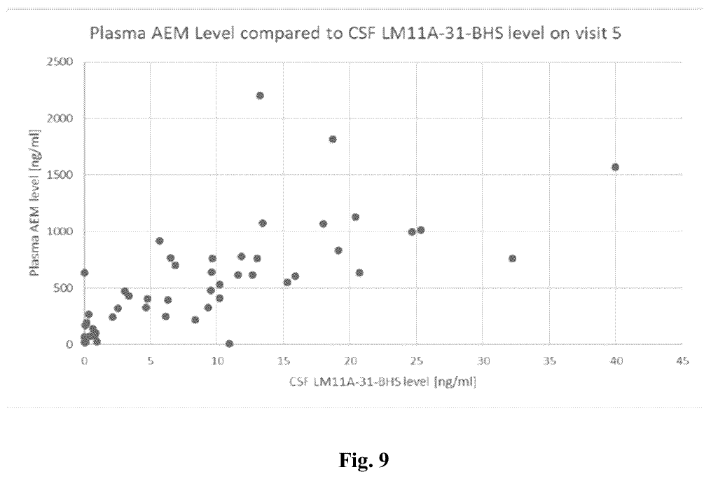

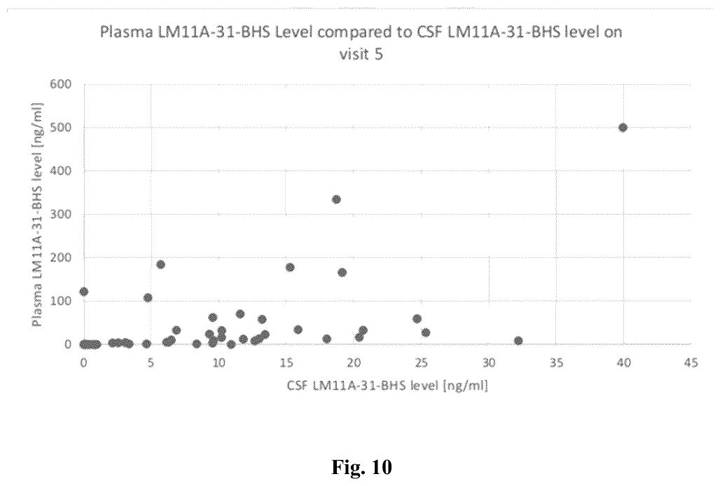

Measurements of the LM11A-31 level and AEM level in plasma (FIGS. 4-7) and, if CSF samples from Visit 5 were available, the LM11A-31 level in CSF (FIG. 8-10).

A 1:1:1 (dose 1: dose 2: control) allocation ratio was used throughout the central randomization process and was structured to allow for a total of at least 240 evaluable patients (80 per group) with center stratification, as only stratification variable, and appropriate block size. During the trial 242 patients were finally randomized, treated and accounted for in the ITT defined population (LM11A31 200 mg bid 78, LM11A31 400 mg bid 83, Placebo control 81 patients). Subsequently, 220 and 211 patients for the Per Protocol and Completers analysis subsets respectively were defined. For all safety evaluations, an overall safety population was defined which included all patients who received at least one dose of medication.

### Patient Disposition

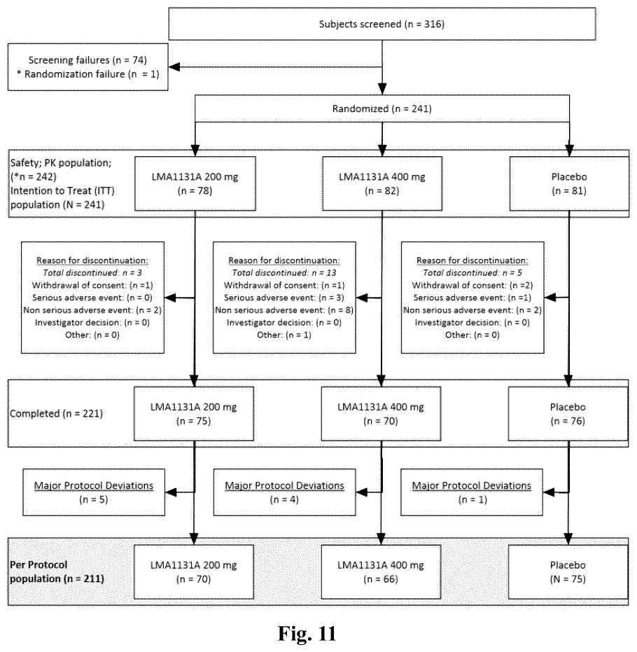

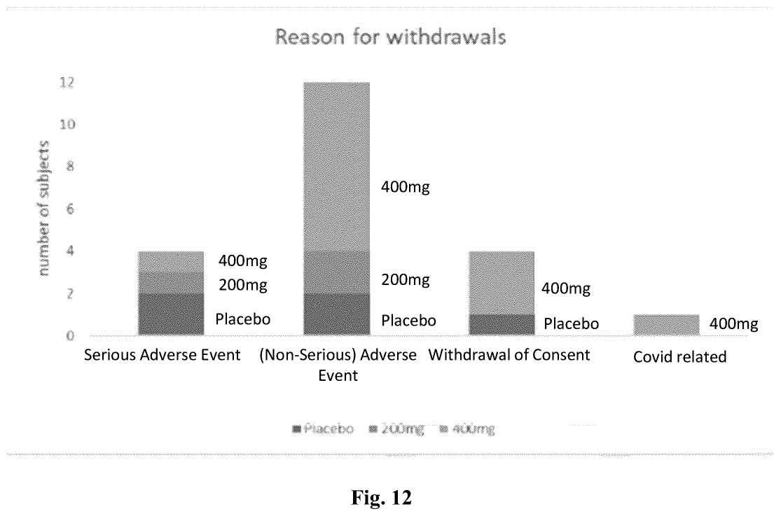

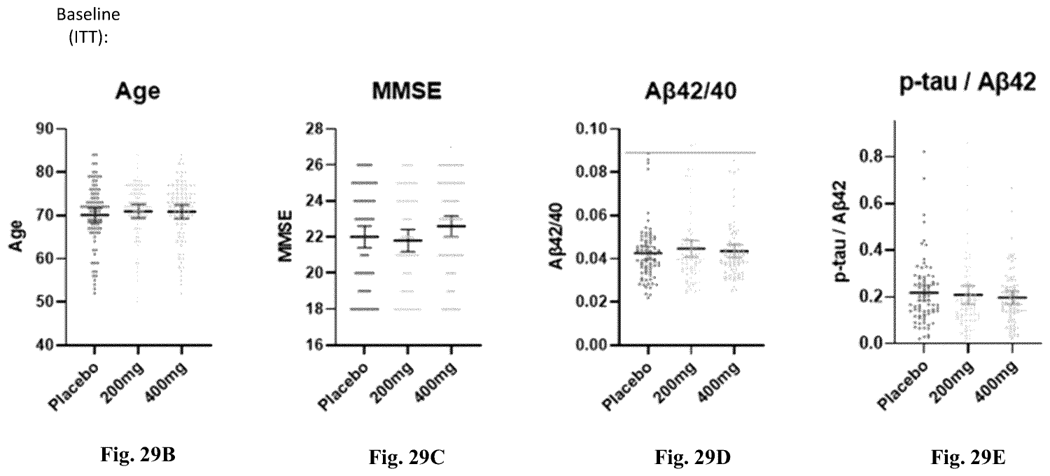

Overall, 316 patients underwent screening and 242 patients were randomized, treated and accounted for in the Safety/PK defined population (LM11A31 200 mg bid 78, LM11A31 400 mg bid 83, Placebo control 81 patients). One subject (LM11A31 400 mg bid) was not properly randomized via the IWRS system and was therefore not included in the ITT population. From the subsets of 221 and 211 patients for the Per Protocol and Completers analysis subsets were subsequently defined. (Table 10, FIG. 11, FIG. 12, and FIG. 29) In FIGS. 29A-29E, the screening process is summarized to reach the 242 subjects that were enrolled into the trial. 221 subjects completed the trial. 20 subjects left the trial for various reasons and 211 subjects completed the trial per protocol. The distribution of enrolled subjects in terms of sex and ApoE genetic status is shown. These distributions across the 3 studies groups are not significantly different, hence the drug effects cannot be ascribed to sex or ApoE differences between the placebo versus drug study groups. Individuals with ApoE4 genetic background have fast rates of decline with Alzheimer's, thus this balance is important. The groups also did not differ with respect to age, MMSE (mini mental status exam—used to define the stage at baseline of the subject), Ab 42/40 ratio and p-tau/Ab42 ratio, another indicator of disease severity at baseline.

TABLE 10
Disposition of patients of the different sites and total

| | | Screening | | |
|---|---|---|---|---|
| Site | Complete | Failure | Withdrawn | TOTAL |
| 101 | 1 | 2 | 1 | 4 |
| 102 | 5 | 2 | 1 | 8 |
| 201 | 33 | 6 | 1 | 40 |
| 202 | 22 | 13 | 4 | 39 |
| 203 | 25 | 6 | 1 | 32 |
| 204 | 18 | 13 | 0 | 31 |
| 301 | 9 | 3 | 1 | 13 |
| 302 | 0 | 0 | 0 | 0 |
| 304 | 15 | 8 | 1 | 24 |
| 305 | 3 | 4 | 0 | 7 |
| 306 | 0 | 0 | 0 | 0 |
| 307 | 0 | 2 | 1 | 3 |
| 308 | 1 | 0 | 0 | 1 |
| 309 | 4 | 2 | 0 | 6 |
| 310 | 12 | 6 | 1 | 19 |
| 401 | 15 | 2 | 4 | 21 |
| 501 | 20 | 1 | 0 | 21 |
| 502 | 3 | 0 | 2 | 5 |
| 503 | 0 | 0 | 0 | 0 |
| 504 | 13 | 1 | 1 | 15 |
| 505 | 22 | 3 | 2 | 27 |
| TOTAL | 221 | 74 | 21 | 316 |

Protocol Deviations

From the disposition details it appears that the study was conducted to a high standard with over 91% (221/242) patients completing and only 21 patients considered as protocol violations. For analysis purposes the participating sites were pooled according to country strata (Spain 62, Czech Republic 104 and Others (Germany, Sweden, Austria) 75 patients) and the demographics across the three treatment groups appeared comparable.

Most of the protocol deviations were related to missing or incomplete tests or missing samples followed by deviations from the visit windows. Due to AD and the high age of the patients the patients were often not able to perform all test required by the protocol. In addition, the COVID-19 pandemic was contributing to the deviations from the visit windows. An overview about major protocol violations is provided in Table 12.

TABLE 11
Protocol deviations listed by country and categorized and normalized per subject

| | Eligibility | Prohibited | Treatment | Compliance | Visit window | | |
|---|---|---|---|---|---|---|---|
| | deviations | medications | deviations | deviations | deviations | Withdrawal | Unblinding |
| Austria | 12.5% | 0.00% | 12.5% | 0.00% | 0.00% | 25.00% | 0.00% |
| Czech | 0.96% | 0.00% | 0.96% | 1.92% | 0.96% | 5.77% | 0.96% |
| Republic | | | | | | | |
| Germany | 4.17% | 6.25% | 0.00% | 4.17% | 0.00% | 8.33% | 0.00% |
| Sweden | 5.26% | 0/00% | 0.00% | 15.79% | 0.00% | 21.05% | 0.00% |
| Spain | 1.59% | 0.00% | 1.59% | 7.94% | 1.59% | 7.94% | 0.00% |

TABLE 12
Major protocol violations (Safety Population)

| | | | | | | | | | | | No of |
|---|---|---|---|---|---|---|---|---|---|---|---|
| | | | | | | Visit | | | Total | Number of | Patients |
| | Site | Eligibility | Prohibited | Treatment | Compliance | Window | | | Number of | Subjects | Enrolled |
| Country | No | Deviation | Deviation | Deviation | Deviation | Deviation | Withdrawals | Unblinding | Events | Involved | per Site |
| Austria | 101 | | | 1 | | | 1 | | 2 | 1 | 2 |
| | 102 | 1 | | | | | 1 | | 2 | 1 | 6 |
| Total | | 1 | 0 | 1 | 0 | 0 | 2 | 0 | 4 | 2 | 8 |
| Czech | 201 | | | 1 | 1 | | 1 | | 3 | 2 | 34 |
| Republic | 202 | | | | | | 4 | | 4 | 4 | 26 |
| | 203 | 1 | | | 1 | 1 | 1 | 3 | 5 | 2 | 26 |
| | 204 | | | | | | | | 0 | 0 | 28 |
| Total | | 1 | 0 | 1 | 2 | 1 | 6 | 3 | 12 | 8 | 104 |
| Germany | 301 | | | | | | 1 | | 1 | 1 | 10 |
| | 304 | 2 | 2 | | 1 | | 1 | | 6 | 2 | 16 |
| | 305 | | | | | | | | 0 | 0 | 3 |
| | 307 | | | | 1 | | 1 | | 2 | 1 | 1 |
| | 308 | | | | | | | | 0 | 0 | 1 |
| | 309 | | | | | | | | 0 | 0 | 4 |
| | 310 | | 1 | | | | 1 | | 2 | 1 | 13 |
| Total | | 2 | 3 | 0 | 2 | 0 | 4 | 0 | 11 | 5 | 48 |
| Sweden | 401 | 1 | | | 3 | | 4 | | 8 | 4 | 19 |
| Total | | 1 | 0 | 0 | 3 | 0 | 4 | 0 | 8 | 4 | 19 |
| Spain | 501 | 1 | | | 2 | | | | 3 | 2 | 20 |
| | 502 | | | | 1 | | 2 | | 3 | 2 | 5 |
| | 504 | | | 1 | | 1 | 1 | | 3 | 2 | 14 |
| | 505 | | | | 2 | | 2 | | 4 | 2 | 24 |
| Total | | 1 | 0 | 1 | 5 | 1 | 5 | 0 | 13 | 8 | 63 |

| | | | | | | | | | | |
|---|---|---|---|---|---|---|---|---|---|---|
| Total | 6 | 3 | 3 | 12 | 2 | 21 | 3 | 48 | 27 | 242 |

### Eligibility Deviations

Overall, 6 eligibility deviations occurred within the complete trial. Single deviations occurred in every country.

### Compliance Deviations

Overall drug compliance was excellent with 93.4% of patients compliant (>=80% prescribed drug taken).

### Withdrawal

Only 21 patients in total within the safety population (n=242) were discontinued. These were, AEs (LM11A31 200 mg bd 2, LM11A31 400 mg bd 8, Placebo control 2 patients), serious AE (LM11A31 200 mg bd 0, LM11A31 400 mg bd 3, Placebo control 1 patients) and Withdrawal of consent (LM11A31 200 mg bd 1, LM11A31 400 mg bd 1, Placebo control 2 patients).

### Rationale and Estimate of Numbers of Subject Required Per Treatment Arm in a Phase 3 Trial of LM11A-31-BHS Conducted for Product Approval and Labeling.

The key factor in determining treatment group size is the measure of clinical effect given that CSF and imaging biomarkers generally exhibit less variation and detect treatment effects at lower n values. There are at least two approaches for estimation of a group size necessary to demonstrate a significant therapeutic effect on the primary clinical outcome measure, in this case ADAS-cog-13.

In the first approach, one can evaluate recent large phase 2 or phase 3 efficacy trials with the major caveat that these studies are largely focused on 18-month endpoints and our phase 2a data is limited to a 26-week endpoint. Another difference is that these recent and current studies generally include prodromal and mild subjects while our trial will include mild-moderate subjects. The Emerge and Trailblazer 2 trials demonstrated slowing of progression by 27% and approximately 35%, respectively, in ADAS-cog-13 scores. In general, within the recent large amyloid antibody trials, differences between placebo and treatment group clinical outcome measures derived at the 18-month time point are 2-3 fold larger than those obtained at the 6-month time points (while the degree of variation at 6- and 18-months is in a similar range). Thus the 30-45% slowing trend noted at 6 months in our phase 2a study points to a potential 18-month effect size similar to, or perhaps greater than, those found in the Emerge and Trailblazer 2 trials. These and some of the other completed or ongoing phase 3 AD trials that include mild stage subjects have treatment group sizes for a given dose in the 500-subject range.

As a second approach, one can perform a power estimation based on the phase 2a study. For ADAS-cog scores, our statistics group calculated (80% power, 5% two-tailed significance), based on the phase 2a ADAS-cog data, that 802 and 616 (ADAS-cog-13 and -11) subjects per treatment arm would be required to demonstrate a significant effect at the 6-month time point. Given the pattern that effect sizes at 18 months tend to be 2-3 time larger than those observed at 18 months, it is estimated that a treatment group size of 500 subjects would be sufficient to detect a significant effect on ADAS-cog-13 with the effect size estimated to be present in the phase 2a trial. It is also anticipated that lowering the maximum age inclusion criteria from 85 that was used in our phase 2a trial to 75 or 80 given that in the mild-moderate population, the higher ages tend to progress at slower rates and hence age range can affect trial design and outcome.

Availability of FDA-approved amyloid antibody treatment will create a need to have additional study subjects. Given that one might estimate that up to a quarter of eligible subjects might be on amyloid antibodies when the phase 3 trial is executed, another approach would be to include 700 subjects per arm with the goal of having approximately 500 subjects per arm that are not on amyloid antibodies and thus ensuring statistical comparisons of drug versus placebo without confounding by antibody treatment. This approach will also provide statistical power to address the secondary question of whether adding LM11A-31-BHS to amyloid antibody treatment might provide additive or synergistic combination therapy effect given the separate mechanisms of action.

### Rationale for Two Drug Doses in a Phase 3 Efficacy Trial Conducted for Product Approval and Labeling.

In the phase 2a trial, in CSF biomarker analyses, for the majority of biomarkers, similar effect sizes were found for both the 200 and 400 mg doses. Analyses for structural MRI remain ongoing. For FDG-PET sROI a greater trend is observed at 400 compared to the 200 mg dose (effect at 400 mg and the difference between 200 and 400 mg doses are not statistically significant). For FDG-PET voxel analysis, 400 mg effect is significant and significantly greater than the 200 mg effect which is significant and significantly greater than the placebo group. In cognitive assessments, trends for slower progression of ADAS-cog and MMSE scores were greater at the 400 compared to 200 mg doses but effects at neither dose reached statistical significance. In the area of adverse events, the rate for several of the adverse events was higher for the 400 mg group and for some events this increase was statistically significant. Of the 16 subjects who discontinued the phase 2a trial due to AEs or SAEs, 11 were from the 400 mg group, 2 from the 200 mg group and 3 from the placebo group. For SAEs, 13 occurred after dosing: 4 placebo, 2 in 200 mg group, 7 in 400 mg group.

For Treatment emergent adverse events (TEAEs): 80 moderate TEAEs occurred with 21, 15 and 61% in the placebo, 200 mg and 400 mg groups. 10 severe TEAEs occurred with 30, 20 and 50% in the placebo, 200 mg and 400 mg groups. For the most common AE of GI side effects (diarrhea in most cases), approximately 35% of subjects in the 400 mg group experienced these side effects while approximately 8% in the 200 mg group.

Overall, it appears that the 400 mg dose has greater efficacy than the 200 mg dose. Given the significantly greater rate of side effects at the high dose it will be advisable to include the 200 mg dose in the event that an excessive number of subjects experience side effects at the high dose. In addition, while the 400 mg dose is also likely to have greater efficacy at the 18-month time point, it will be a high priority to confirm, with the statistical power of a phase 3 trial, their relative efficacies given the safety profiles.

### Data Quality Assurance (Phase 2a Trial)

The study design, conduct, recording, reporting and archiving of all relevant documents/data was done in accordance with GCP.

An electronic CRF (eCRF, Data Magik Limited, Salisbury, UK) was completed for each patient having entered Visit 1 and thus being screened.

The Kapture software was used for capturing and processing the data generated. The system generated an audit trail tracking every action (e.g., data entry, data modification, query raising, allocating, resolving) taken within the system. All individuals using the Kapture software were accountable and responsible for all actions initiated by their electronic signature. Access attempts to the Kapture software were monitored and appropriate actions were taken in the event of any unauthorized access attempts, or user problems with electronic signatures and logging in. User groups (e.g., Investigators, Site Staff, CRA) received appropriate, documented user training provided by the project manager. Personnel authorized for data entry (e.g., Investigator and Site Staff) were responsible for completing the eCRF in English and had to enter all clinical trial data for every patient in the indicated order. The principal investigator signed and dated the patients' eCRF after completion.

The information collected and entered in the eCRFs had to match the source documents. Once the database was frozen, completed eCRFs were saved on a DVD in pdf format and archived together with the ISF from each site.

### Monitoring

Monitoring Visits were performed by a monitor of the CRO. The trial site was monitored by means of on-site Visits and regular inspection of CRF with sufficient frequency to perform source data verification and ensure compliance with protocol procedures. The monitor advised the investigator regarding the practical conduct of the clinical trial and assisted him/her in working according to the protocol, GCP and regulatory requirements. The trial site/investigator guaranteed direct access to the source documents. Verification of data against original source documents and query resolution was conducted for 100% for the first two patients randomized at the site. For 10% of patients subsequently randomized also 100% source data verification (SDV) had to be performed, however, if data quality would have been not acceptable, extended SDV should have been done until an acceptable level of data quality was achieved. For screening failures, only capability of giving IC and correct completion of informed consent forms (ICF) were be reviewed by the CRA. For all patients who discontinued trial medication 100% SDV on study termination Visit should have been done. Standard monitoring reports were produced after each monitoring Visit. Additional internal study checks were performed by the QA personnel of the CRO.

### Audit and Inspection

Regulatory Authority may carry out an inspection during the clinical trial and/or after trial completion. Also trial-related audits by auditors mandated by PharmatrophiX Inc. are possible. Therefore, direct access to source data/documentation will be provided for audits, for review by the Ethical Committee and for any regulatory inspection.

### Confidentiality and Data Protection

The investigator assures that only authorized persons have access to the patient's personal data. Patients can only be identified by the enrolment log in correspondence with the Subject ID on CRFs. All data contained in the patients' medical history are considered confidential.

### Datasets Analyzed

Four main patient populations provide the basis for all statistical analyses, data evaluations and summaries included in the investigation.

The primary patient population of interest is the ‘Intention to Treat’ population (ITT). This includes all patients who received at least one dose of medication and who subsequently provide any post baseline information. All patients were analyzed according to the treatment they were scheduled to receive.

A secondary supporting population is also investigated. This is defined as the ‘Per-Protocol’ completers population (PPc) and this includes all patients who satisfactorily completed the 26-week treatment period and fully comply with requirements of the protocol regarding the exploratory outcome evaluations. Any major protocol violators were excluded. All patients in this group are analyzed according to the treatment they actually receive.

Furthermore, a sensitivity analysis was carried out using the ‘Per Protocol’ population but including patients who have withdrawn early. This is defined as the PP non-completers population set (PPn).

For all safety evaluations, only an overall ‘Safety’ population is described. This includes all patients who received at least one dose of medication and consider all patients according to the treatment they actually receive. Additionally, a PK population will be described for only patients receiving active study IMP.

### Demographic and Other Baseline Characteristics

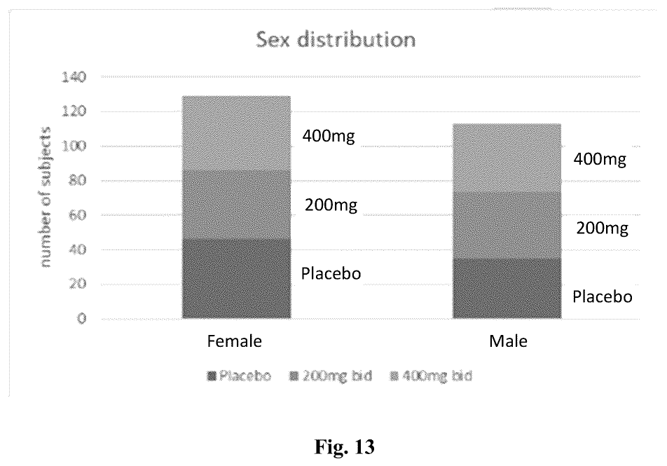

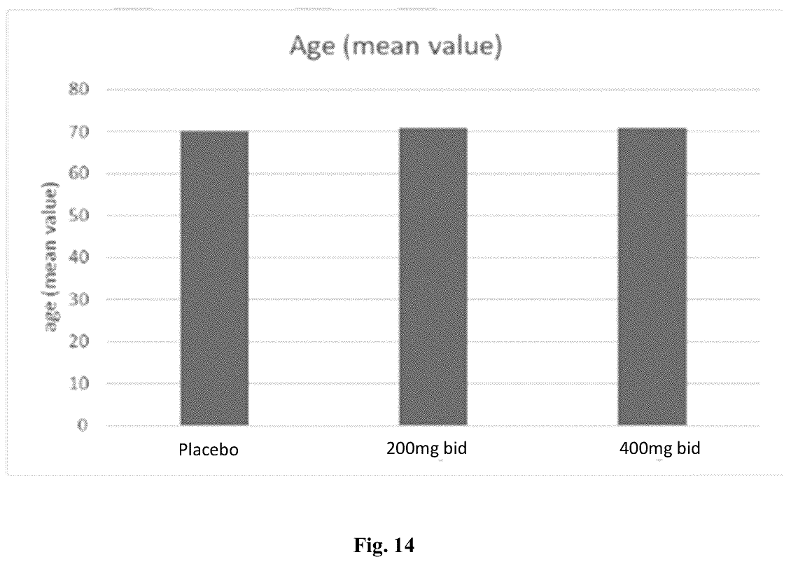

Demographics and baseline characteristics recorded at screening (Visit 1) were summarized using descriptive statistics. The uniform distribution of sex and age within the three treatment groups is visible on FIG. 13 and FIG. 14. Further demographic information is show in Table 13. The race distribution is not shown due to 100% white within this trial.

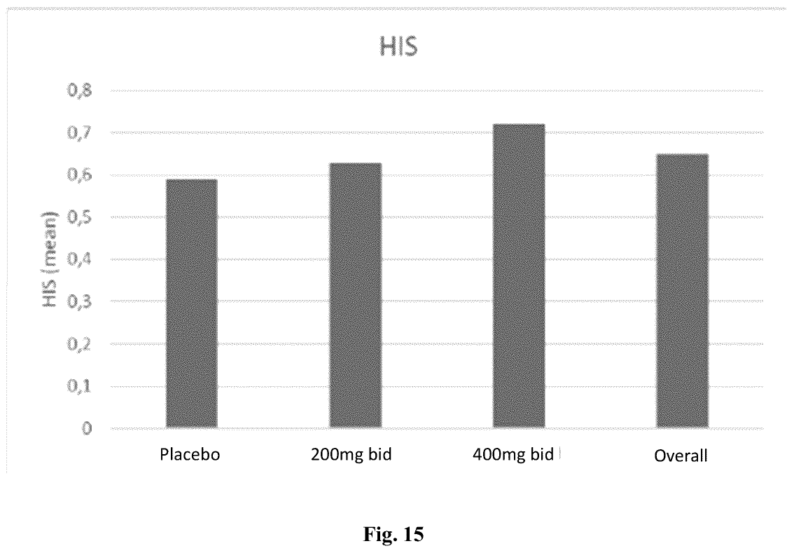

The Hachinski Ischemic Scale (Moroney et al., 1997) (HIS) is increasing within the three groups within the safety population leading to a slight skewness lightly discriminating the dosage groups over the placebo group. (FIG. 15)

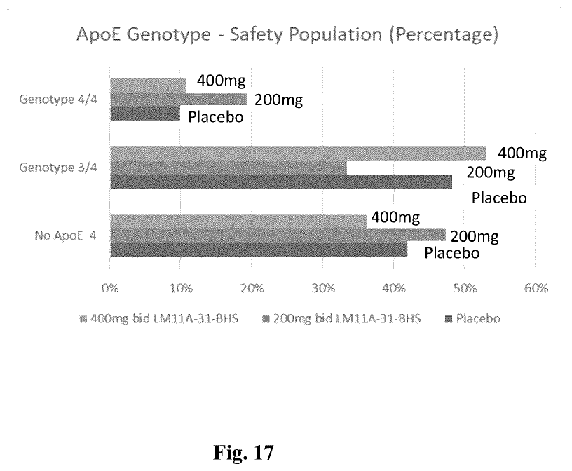

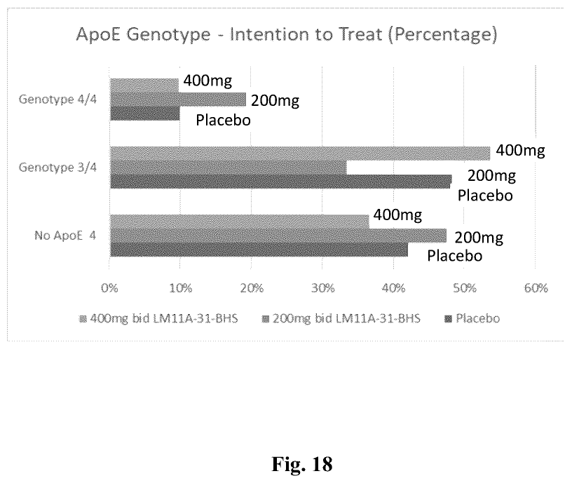

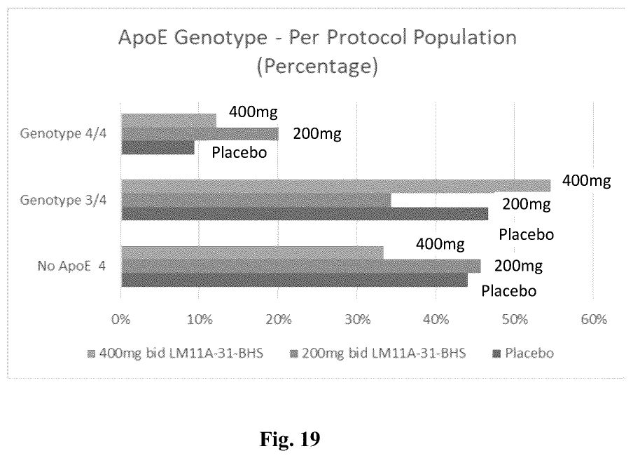

The distribution of the ApoE genotype within the groups reveals a slightly higher percentage of ApoE4 carriers within the highest dosage group and a slight decrease of the ApoE4 carrier percentage within the lower dosage group in the Safety Population. The placebo group has almost the intermediate distribution of the ApoE4 genotype within the ITT Population. Separated to the different genotypes of the ApoE4 carriers the lowest dosage group has the highest number of ApoE 4/4 carriers with almost doubled ratio compared to the residual groups. (See FIG. 17 and Table 14). For the ITT and PP population the percentage of the ApoE 4/4 carriers remains elevated and even increases for the lowest dosage group. (FIG. 18, FIG. 19, Table 14 and Table 15)

TABLE 13
Summary of demographic characteristics of participants in phase IIa trial

| | | | | | LM11A | | LM11A | | | |
|---|---|---|---|---|---|---|---|---|---|---|
| | | | Placebo | | 200 mg bid | | 400 mg bid | | Overall | |
| Observation | | Statistic | (N = 81) | | (N = 78) | | (N = 83) | | (N = 242) | |
| Sex | Female | n (%) | 46 | (56.8) | 40 | (51.3) | 43 | (51.8) | 129 | (53.3) |
| | Male | n (%) | 35 | (43.2) | 38 | (48.7) | 40 | (48.2) | 113 | (46.7) |
| Race | White | n (%) | 81 | (100.0) | 78 | (100.0) | 83 | (100.0) | 242 | (100.0 |
| Domicilliary | Own | n (%) | 81 | (100.0) | 78 | (100.0) | 83 | (100.0) | 242 | (100.0 |
| Status | Home/ | | | | | | | | | |
| | Relative | | | | | | | | | |
| Age at | | N | 81 | | 78 | | 83 | | 242 | |
| Informed | | | | | | | | | | |
| Consent | | | | | | | | | | |

| | | | | |
|---|---|---|---|---|
| MEAN | 70.19 | 70.92 | 70.90 | 70.67 |
| SD | 7.49 | 6.93 | 7.14 | 7.17 |
| MEDIAN | 72.00 | 72.00 | 72.00 | 72.00 |
| LQ | 67.00 | 68.00 | 67.00 | 67.00 |
| UQ | 75.00 | 76.00 | 76.00 | 76.00 |
| MINIMUM | 52.00 | 50.00 | 52.00 | 50.00 |
| MAXIMUM | 84.00 | 84.00 | 84.00 | 84.00 |

TABLE 14
Percentual distribution of baseline characteristics (ITT)

| | | 200 mg bid | 400 mg bid |
|---|---|---|---|
| | | LM11A-31- | LM11A-31- |
| | Placebo | BHS | BHS |
| MMSE severity - mild | 61.73% | 51.28% | 62.20% |
| MMSE severity - moderate | 38.27% | 48.72% | 37.80% |
| ApoE 2/4 | 0.00% | 2.56% | 2.44% |
| ApoE 3/4 | 48.15% | 30.77% | 51.22% |
| ApoE 4/4 | 9.88% | 19.23% | 9.76% |
| No ApoE 4 | 41.98% | 47.44% | 36.59% |
| Sex - Female | 56.79% | 51.28% | 51.22% |
| Sex - Male | 43.21% | 48.72% | 48.78% |
| Age group - 50-59 | 14.67% | 11.43% | 12.12% |
| Age group - 60-69 | 29.33% | 18.57% | 27.27% |
| Age group - 70-79 | 56.00% | 77.14% | 74.24% |
| Age group - 80-89 | 8.00% | 4.29% | 10.61% |

TABLE 15
Percentual distribution of baseline characteristics (PP)

| | | 200 mg bid | 400 mg bid |
|---|---|---|---|
| | | LM11A-31- | LM11A-31- |
| | Placebo | BHS | BHS |
| MMSE severity - mild | 62.67% | 54.29% | 56.06% |
| MMSE severity - moderate | 37.33% | 45.71% | 43.94% |
| ApoE 2/4 | 0.00% | 2.86% | 2.89% |
| ApoE 3/4 | 46.67% | 31.43% | 52.24% |
| ApoE 4/4 | 9.33% | 20.00% | 11.94% |
| No ApoE 4 | 44.00% | 45.71% | 32.84% |
| Sex - Female | 57.33% | 50.00% | 53.03% |
| Sex - Male | 42.67% | 50.00% | 46.97% |
| Age group - 50-59 | 13.33% | 8.57% | 9.09% |
| Age group - 60-69 | 29.33% | 17.14% | 24.24% |
| Age group - 70-79 | 50.67% | 71.43% | 59.09% |
| Age group - 80-89 | 6.67% | 2.86% | 7.58% |

TABLE 16
Different ApoE genotype distribution within groups

| | | No | | |
|---|---|---|---|---|
| | | ApoE 4 | ApoE 3/4 | ApoE 4/4 |
| Safety | Placebo | 41.98% | 48.15% | 9.88% |
| population | 200 mg bid | 47.44% | 33.33% | 19.23% |
| | LM11A-31-BHS | | | |
| | 400 mg bid | 36.14% | 53.01% | 10.84% |
| | LM11A-31-BHS | | | |
| Intention to | Placebo | 41.98% | 48.15% | 9.88% |
| treat | 200 mg bid | 47.44% | 33.33% | 19.23% |
| population | LM11A-31-BHS | | | |
| | 400 mg bid | 36.59% | 53.66% | 9.76% |
| | LM11A-31-BHS | | | |
| Per protocol | Placebo | 44.00% | 46.67% | 9.33% |
| population | 200 mg bid | 45.71% | 34.29% | 20.00% |
| | LM11A-31-BHS | | | |
| | 400 mg bid | 33.33% | 54.55% | 12.12% |
| | LM11A-31-BHS | | | |

The Hachinski Ischemic Scale (HIS) is increasing within the three groups within the safety population leading to a slight skewness lightly discriminating the dosage groups over the placebo group (FIG. 15).

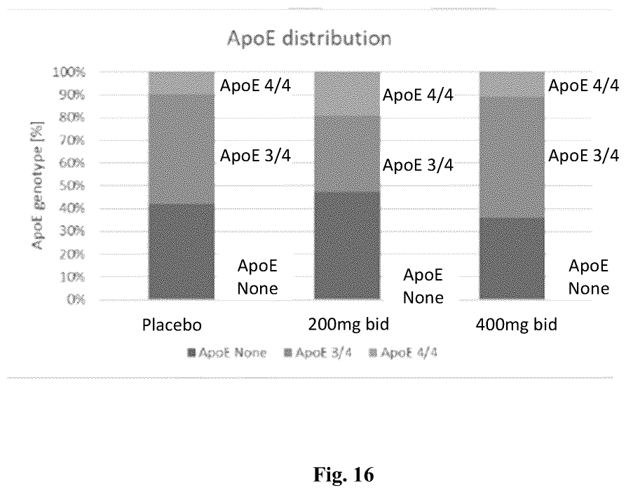

The distribution of the ApoE genotype within the groups revealed a slightly higher percentage of ApoE4 carriers within the highest dosage group and a slight decrease of the ApoE4 carrier percentage within the lower dosage group in the safety population. The placebo group had almost the intermediate distribution of the ApoE4 genotype within the safety population. Separated to the different genotypes of the ApoE4 carriers, the lowest dosage group had the highest number of ApoE 4/4 carriers with almost doubled ratio compared to the residual groups (FIG. 16).

### Measurement of Treatment Compliance

At each assessment Visit the number of capsules dispensed and returned was recorded together with the dates of issue and return. At Visits 3, 4 and 5 (Week 4, 12 and 26 or Early Discontinuation) the actual total number of capsules taken (capsules dispensed—capsules returned) was summed for the entire treatment period. Using the overall start date and end date of treatment the required total number of capsules (end date—start date)*4) was derived and used to express overall treatment compliance as a percentage ((Actual/Required)*100). A ‘cut off’ of >=80% drug taken was used to classify Compliant/Non-Compliant patients. The overall compliance within the different dosage groups was above 90%. The highest number of subjects below the compliance threshold was found in the highest dosage group followed by the low dosage group. Overall, 91.3% (221/242) patients satisfactorily completed the study and only 21 patients were considered protocol violations and excluded from the Per Protocol population. The main reasons for exclusion were <80% compliance or ‘Off’ treatment for more than 3 days (14/242 patients: 5.8%).

### Statistical/Analytical Observations

All data will be described and analyzed according to Endpoint or Assessment Visit (Screening, Week 0, 4, 12, 26), treatment group (LM11A-31 200 mg bid/400 mg bid/placebo) and Overall for each patient population (Safety, ITT, PP).

### Handling of Dropouts or Missing Data

All possible efforts were be made to ensure that patients completed the required assessments at each Visit and in general there was no imputation of any missing data for any of the assessments performed.

To allow for patient attrition or completely missing patient assessments a ‘Last observation performed’ assessment was derived for all exploratory outcome and safety variables. This approach ensured use of the last available none missing post baseline value for all patients and is consistent with the randomization and principal of Intention-To-Treat.

### Analysis of Safety and Exploratory Endpoints

With respect to vital signs, no relevant changes could be identified within the safety population. For heart rate and body temperature no clinically significant changes were measured during the visits.

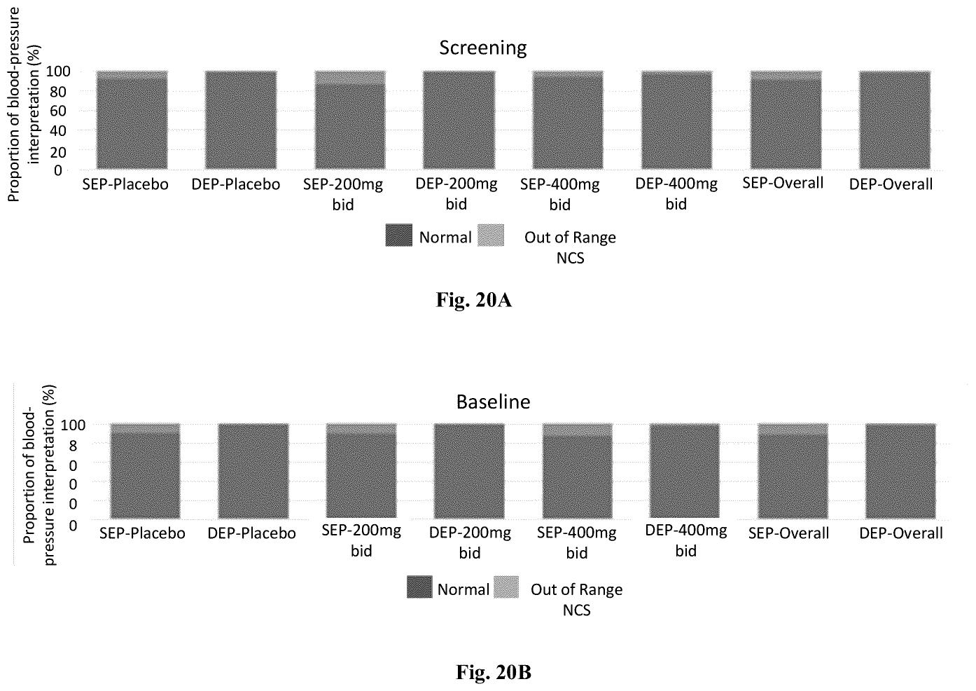

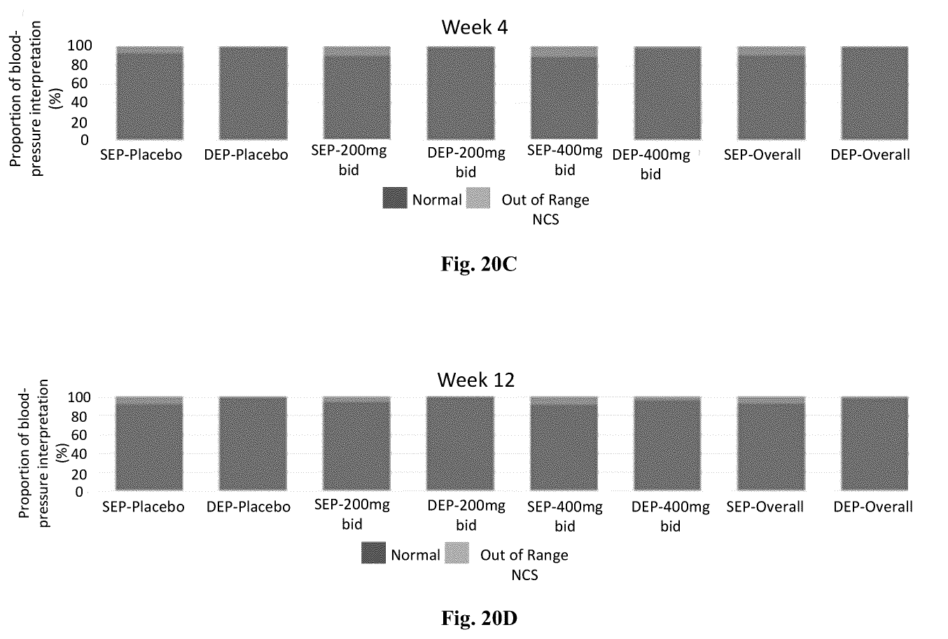

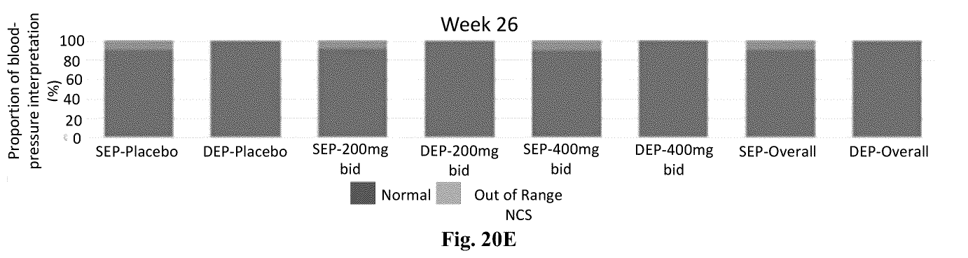

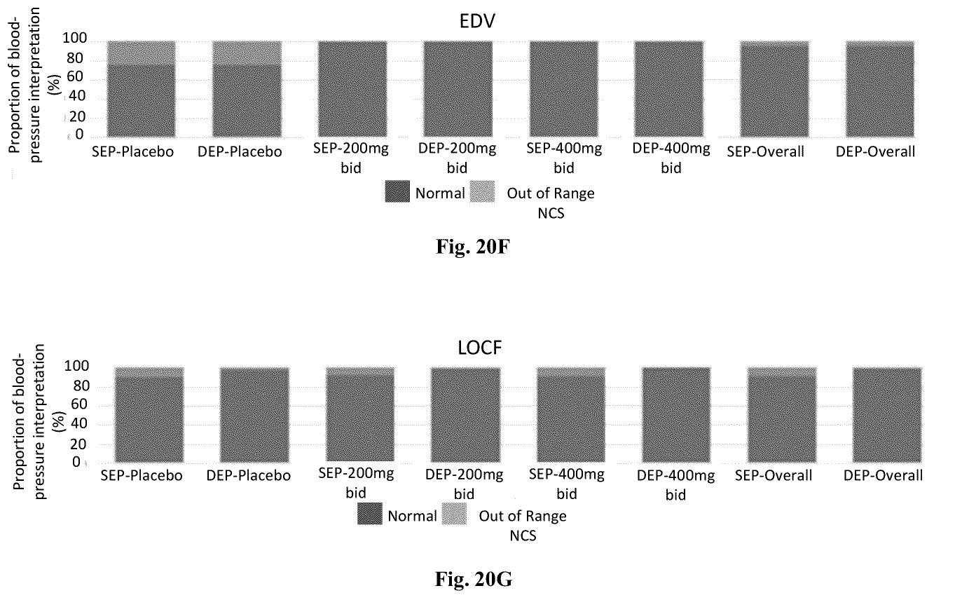

With respect to blood pressure, no relevant changes could be identified within the safety population as seen in FIGS. 20A-20G.

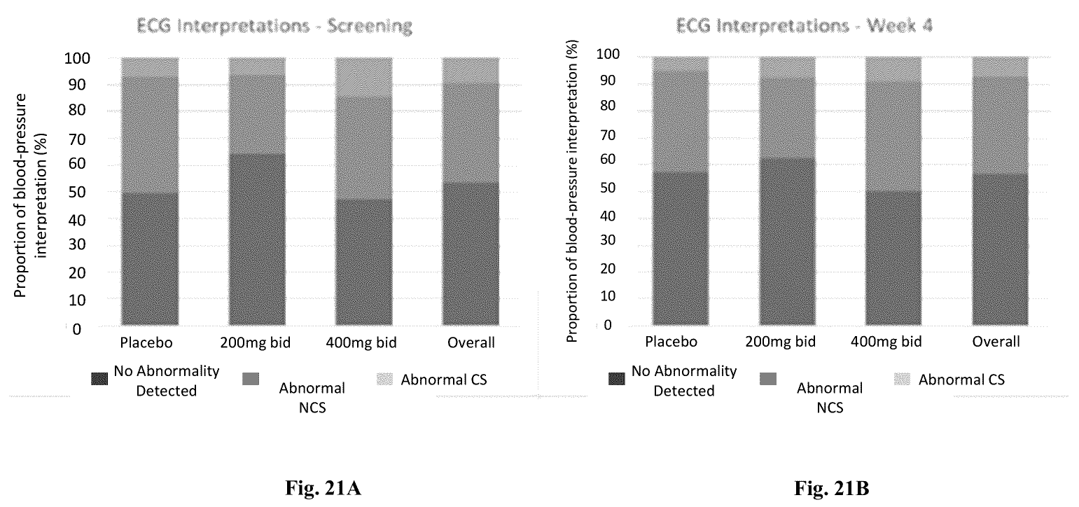

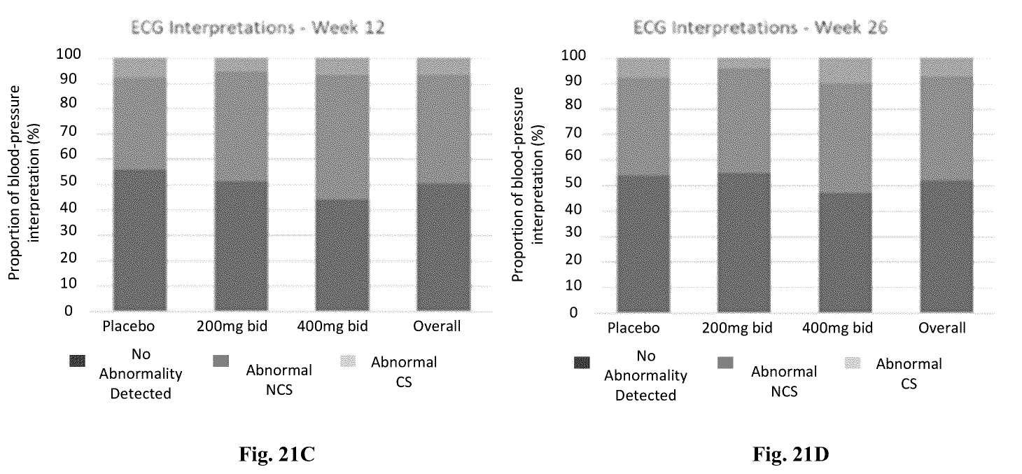

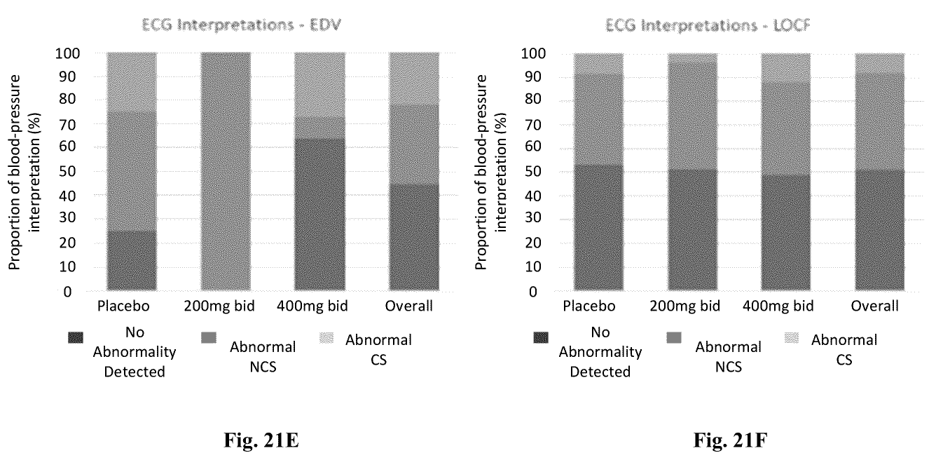

With respect to 12-lead ECG, no relevant changes could be identified within the safety population as seen in FIGS. 21A-21E.

MRI-based safety analysis found no drug related adverse outcomes including hemorrhage or edema.

The C—SSRS scans were performed at the screening and the baseline visit and the results are listed within Table 17.

TABLE 17
C-SSRS scans performed during phase IIa trials

| | | LM11A | LM11A | |
|---|---|---|---|---|
| | | 200 mg | 400 mg | |
| | Placebo | bid | bid | Overall |
| SCREENING | (N = 81) | (N = 78) | (N = 83) | (N = 242) |
| Suicidal Ideation | | 1 (1.3) | 2 (2.4) | 3 (1.2) |
| Wish to be Dead | | 1 (1.3) | 2 (2.4) | 3 (1.2) |
| Non-specific Active Suicidal Thoughts | | 1 (1.3) | 1 (1.2) | 2 (0.8) |
| Active suicidal ideation with Any Methods (Not Plan) | | | | |
| without Intent to Act | | | | |
| Active Suicidal Ideation with Some Intent to Act | | | | |
| without Specific Plan | | | | |
| Active Suicidal Ideation with Specific Plan and Intent | | | | |
| Suicidal Behavior | | | | |
| Preparatory Acts or Behavior | | | | |
| Aborted Attempt | | | | |
| Interrupted Attempt | | | | |
| Non-fatal Suicide Attempt | | | | |
| Self-injurious Behavior without Suicidal Intent | | | | |
| BASELINE (VISIT 2) | | | | |
| Suicidal Ideation | 1 (1.2) | 1 (1.3) | 2 (2.4) | 4 (1.7) |
| Wish to be Dead | 1 (1.2) | 1 (1.3) | 2 (2.4) | 4 (1.7) |
| Non-specific Active Suicidal Thoughts | | 1 (1.3) | | 1 (0.4) |
| Active suicidal ideation with Any Methods (Not Plan) | | | | |
| without Intent to Act | | | | |
| Active Suicidal Ideation with Some Intent to Act | | | | |
| without Specific Plan | | | | |
| Active Suicidal Ideation with Specific Plan and Intent | | | | |
| Suicidal Behavior | | | | |
| Preparatory Acts or Behavior | | | | |
| Aborted Attempt | | | | |
| Interrupted Attempt | | | | |
| Non-fatal Suicide Attempt | | | | |
| Self-injurious Behavior without Suicidal Intent | | | | |

### Adverse Events

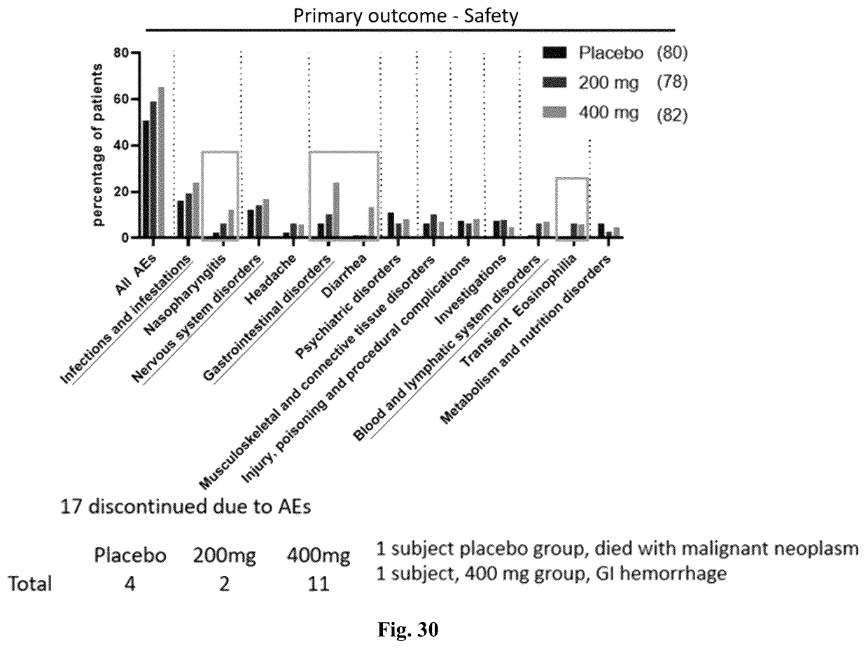

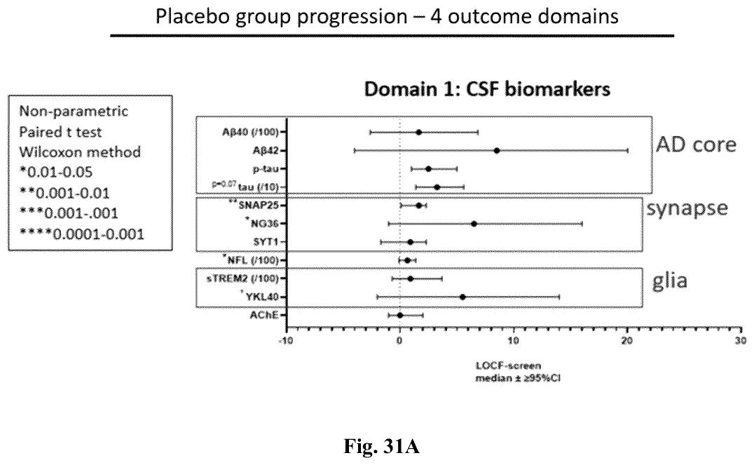

A total of 33 patients (14%) had adverse events considered to be related to LM11A-31. Twelve patients (15%) received LM11A-31 200 mg, 13 patients (15%) received LM11A-31 400 mg and 8 patients (10%) received placebo control. A total of 118 patients (49%) had mild (Grade 1) adverse events, of which 40 patients (51%) received LM11A-31 200 mg, 46 patients (55%) received LM11A-31 400 mg and 32 patients (40%) received placebo control. A total of 46 patients (19%) had moderate (Grade 2) adverse events, of which 11 patients (14%) received LM11A-31 200 mg, 24 patients (290) received LM11A-31 400 mg and 11 patients (14%) received placebo control. A total of 10 patients (48) had severe (Grade 3) adverse events, of which 2 patients (3%) received LM11A-31 200 mg, 5 patients (6%) received LM1 1A-31 400 mg and 3 patients (4%) received placebo control. There were no life-threatening (Grade 4) adverse events reported in any group, and there was one fatal (Grade 5) adverse event reported in the placebo control group. Adverse events are summarized in Table 18 and primary outcome related with AEs is shown in FIG. 30. For the safety results of FIG. 30, 17 subjects left the trial because of adverse events (AEs). AEs were similar between placebo and drug groups except for higher rates in the drug group of nasopharyngitis, GI symptoms including diarrhea and transient, asymptomatic blood eosinophilia.

TABLE 18
Summary of adverse events observed during phase IIa trial

| | | | | | LM11A | | | LM11A | | | | | |
|---|---|---|---|---|---|---|---|---|---|---|---|---|---|
| | | Placebo | | | 200 mg bid | | | 400 mg bid | | | Overall | | |
| Adverse event | | (N = 81) | | | (N = 78) | | | (N = 83) | | | (N = 242) | | |
| category | | n | % | freq | n | % | freq | n | % | freq | n | % | Freq |
| All Adverse Events | | 42 | 51.9 | 100 | 47 | 60.3 | 109 | 55 | 66.3 | 185 | 144 | 59.5 | 394 |
| Pre-Treatment Signs | | 7 | 8.6 | 9 | 8 | 10.3 | 9 | 8 | 9.6 | 12 | 23 | 9.5 | 30 |
| and Symptoms (PTSS) | | | | | | | | | | | | | |
| Treatment Emergent | | 41 | 50.6 | 91 | 46 | 59.0 | 100 | 54 | 65.1 | 173 | 141 | 58.3 | 364 |
| Adverse Events | | | | | | | | | | | | | |
| (TEAEs) | | | | | | | | | | | | | |
| Drug | Related | 8 | 9.9 | 11 | 12 | 15.4 | 15 | 13 | 15.7 | 23 | 33 | 13.6 | 49 |
| Relationship | Not | 36 | 44.4 | 80 | 41 | 52.6 | 85 | 48 | 57.8 | 150 | 125 | 51.7 | 315 |
| | Related | | | | | | | | | | | | |
| Intensity | Mild | 32 | 39.5 | 69 | 40 | 51.3 | 86 | 46 | 55.4 | 119 | 118 | 48.8 | 274 |
| | Moderate | 11 | 13.6 | 19 | 11 | 14.1 | 12 | 24 | 28.9 | 49 | 46 | 19.0 | 80 |
| | Severe | 3 | 3.7 | 3 | 2 | 2.6 | 2 | 5 | 6.0 | 5 | 10 | 4.1 | 10 |

| AEs Leading To | 4 | 4.9 | 9 | 5 | 6.4 | 5 | 15 | 18.1 | 20 | 24 | 9.9 | 34 |
|---|---|---|---|---|---|---|---|---|---|---|---|---|
| Discontinuation of | | | | | | | | | | | | |
| Study IMP | | | | | | | | | | | | |
| Serious AEs | 4 | 4.9 | 4 | 2 | 2.6 | 2 | 7 | 8.4 | 7 | 13 | 5.4 | 13 |
| AEs Leading To Death | 1 | 1.2 | 1 | | | | | | | 1 | 0.4 | 1 |

The TEAEs reported by frequency show the most frequently reported were Nasopharyngitis (17 patients), Diarrhea (13 patients), Headache (12 patients) and Eosinophilia (10 patients).

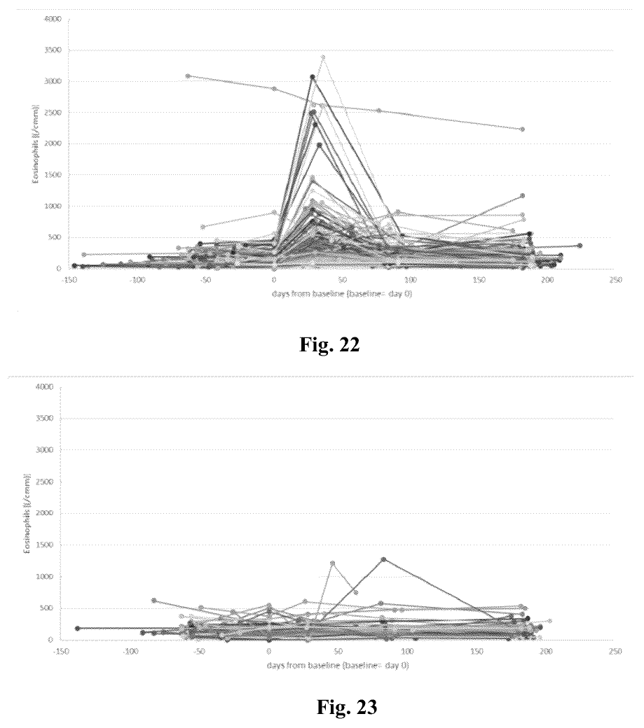

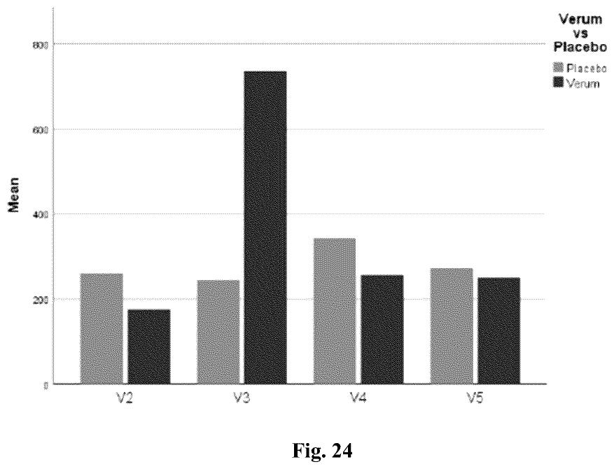

Within the trial, no increase of eosinophils of the treatment group was classified as a serious adverse event, the eosinophil level never reached a value over 5000 per μl that could have been classified as a severe increase, and no combined symptoms gave any indication of a drug induced hypersensitivity syndrome. In total, 10 subjects receiving treatment had eosinophilia reported as an adverse event. In none of these cases was the increase in eosinophil levels thought to be associated with related clinical symptoms. In cases in which treatment was continued, the increase in eosinophil level was found to be transient. Overall, 73 subjects within the verum group had eosinophil counts transiently above 500 per μl. In comparison to the subjects receiving a treatment with the salt of Formula (II), subjects treated with placebo showed no changes of eosinophil values (FIGS. 12-24).

This example illustrates the safety and tolerability of the salt of Formula (II). Overall, no serious adverse events occurred that raised concerns about the tolerability of the drug.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### Example 14. Observations of Data

No significant changes within the different groups and analyzed populations could be identified for the vital signs, the blood pressure, the 12 lead ECG.

The analyzed MRI data shows no events that raised any concern about the drug safety. The safety laboratory and the urine analysis did not show any patterns of concern. The only statistically significant difference for the treatment groups was an increased eosinophil value that reached values that were considered as clinically significant (11 subjects at week 4) but never reached any severe state and was not associated with any assignable symptoms. Furthermore, all increased eosinophil values either considered as clinically significant or non-clinically significant declined to normal levels again in the following Visits independently from stopping the dosage of the IMP or not. Nevertheless, future trials need to pay special attention to the eosinophils values to uphold and guarantee the safety of the subjects. Overall, no results raised any concerns about the safety of LM11A-31-BHS.

Preclinical studies in mice point to degenerative mechanisms that occur in Alzheimer's disease (AD) and in mouse models of AD and the human biomarkers that can be used to assess these mechanisms as shown in FIG. 26. In published mouse studies involving amyloid based mice (L/S-APP) or tau based mice (PS19), LM11A-31 inhibits levels of pathological tau, loss of neurites, loss of synapses and spines, activation of astrocytes (GFAP staining is less) and activation of microglia (CD68 staining is less) and corrects synaptic function and hence the loss of LTP signal and measured by EPSP slope in hippocampal slices. Human CSF levels of p-tau indicate degree of pathological tau. Human structural MRI (sMRI) indicates degree of gray matter brain atrophy which occurs in the setting of loss of neurites and synapses. Human CSF levels of synaptotagmin-1 (SYNT-1) and SNAP-25 indicate degree of degeneration of presynaptic elements of synapses; and CSF levels of neurogranin (NG) indicate degree of degeneration of post-synaptic elements, especially dendritic spines. Elevations in CSF levels of YKL-40 and sTREM2 indicate increased activation of astrocytes and microglia, respectively. The term “glia” refers to both astrocytes and microglia. sROI and voxel based FDG-PET is used to measure synaptic function in humans.

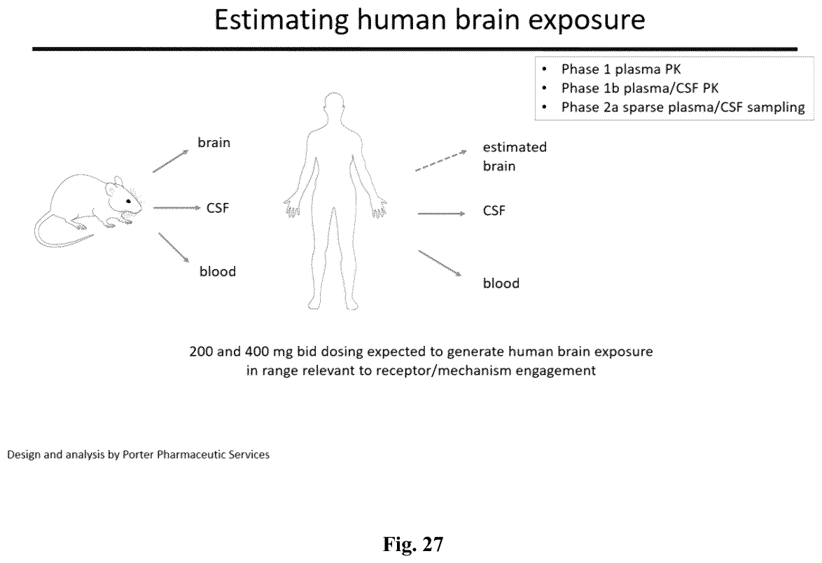

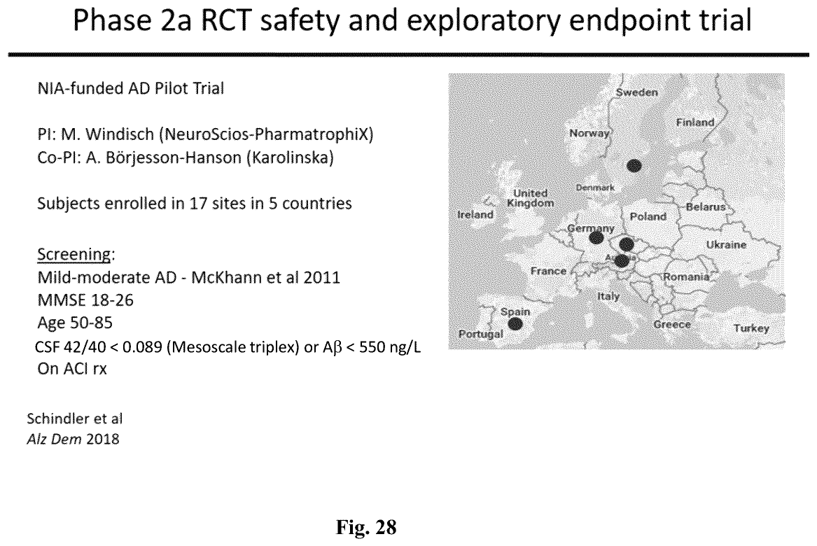

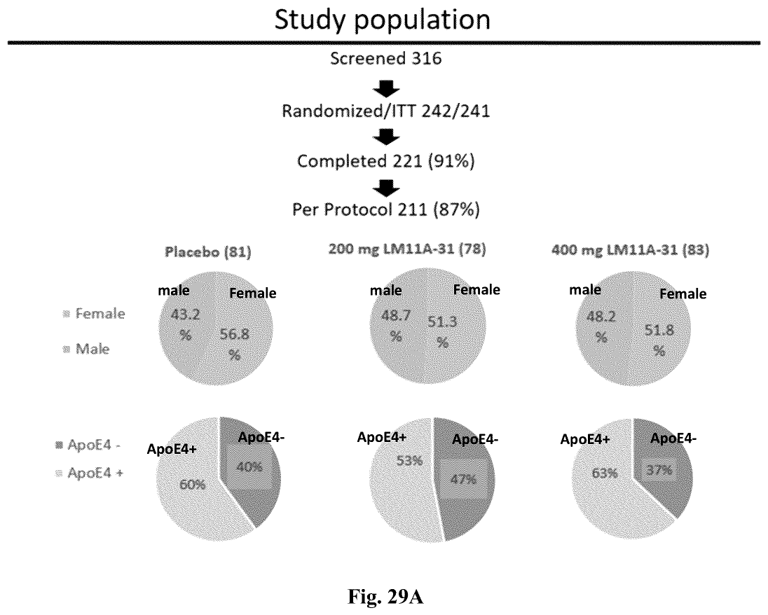

FIG. 27 shows pharmacokinetics studies in mice including measurement of drug levels in blood, CSF and brain tissue and studies done in the 3 listed human studies in which drug levels were measured in plasma in phase 1 and in plasma and CSF in the 1b and 2a study allow the first establishment that oral dosing at 200 mg bid and 400 mg bid lead to estimated human brain levels of drug that are in a range found to engage target mechanisms and derive therapeutic benefit in mouse models. Key inclusion criteria for the 2a trial include a diagnosis of mild-moderate AD by McKhann criteria and a CSF measure of Ab 42/40 ratio under 0.089 OR a Ab 42 level <550 ng/L (FIG. 28). These criteria are discussed in Schindler et al. 2018.

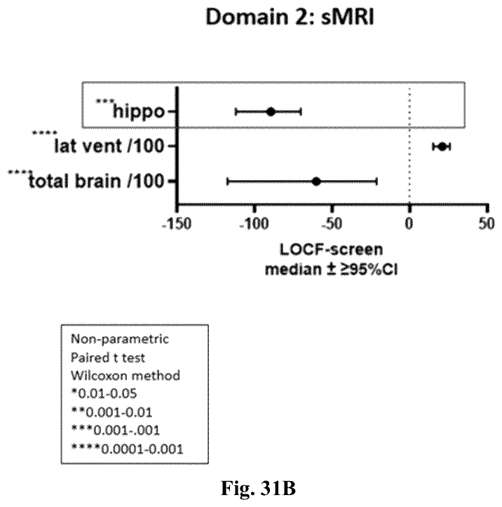

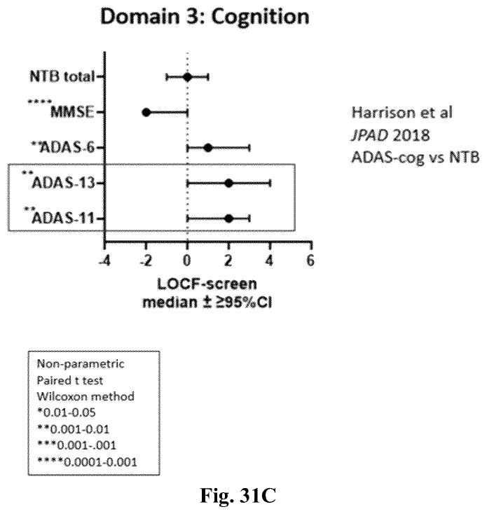

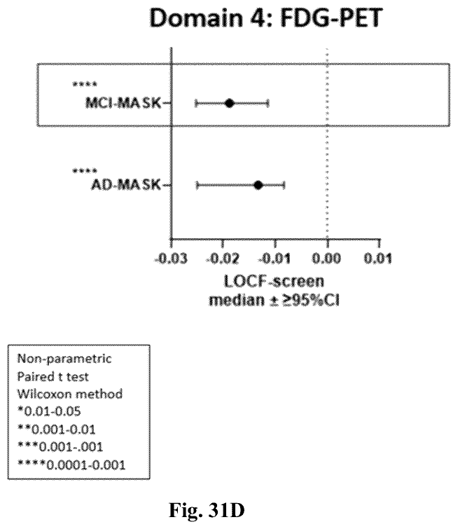

FIGS. 31A-31D demonstrate the progression of the placebo group in the population of mild-moderate AD over the 26-week study period. Many of the outcome measures showed statistically significant progression. Longitudinal progression of CSF biomarkers in the patient population over a 6-month period has not been studied well. This information is not obvious but is critical in terms of knowing what to evaluate in the drug groups. Bars moving to the right indicated an increase and for the measures other than Ab, indicating increased degeneration. For MRI, it is shown that the expected decreased volume (i.e. atrophy) of the hippocampus and total brain, for lateral ventricles and increased volume which goes along with brain atrophy. Similarly, for the FDG-PET measures, progression over a 6-month period is not well studied; the shift to the left is the expected loss of metabolic function which indicates loss of synaptic function. Progression in terms of cognition and sMRI is better studied but results are not consistent across studies. Therefore, assessment within this specific group of subjects is critical and would not be able to be predicted. For ADAS cognition, it is shown that the shift to the right of an increased score which is a worsening. For MMSE, the shift to the left is also a worsening. NTB did not change and in the population was not useful.

Exploratory Outcomes for Domain 1 Related with CSF AD Core Biomarkers

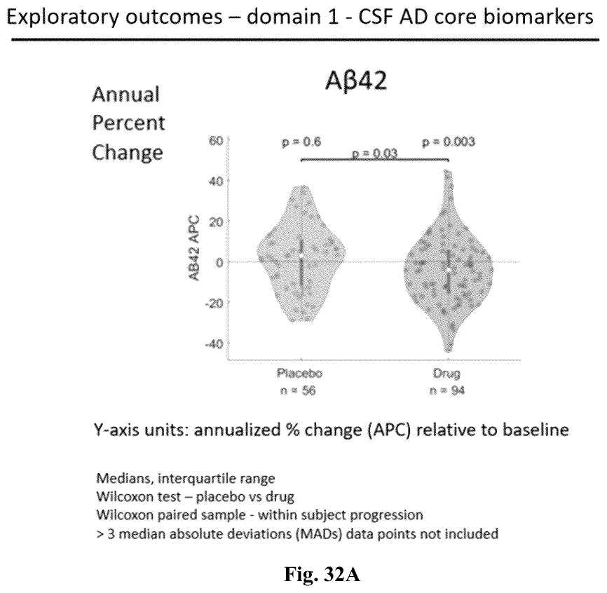

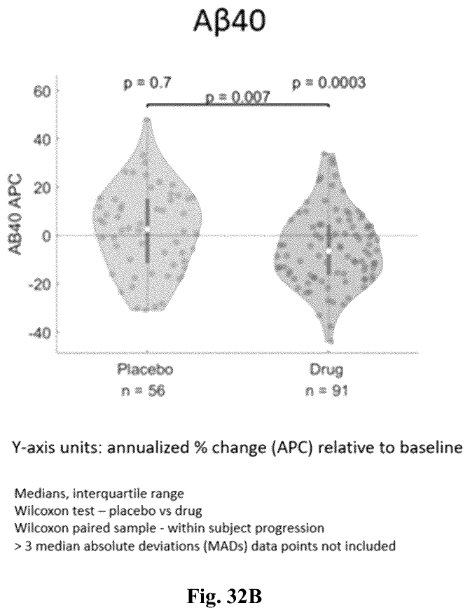

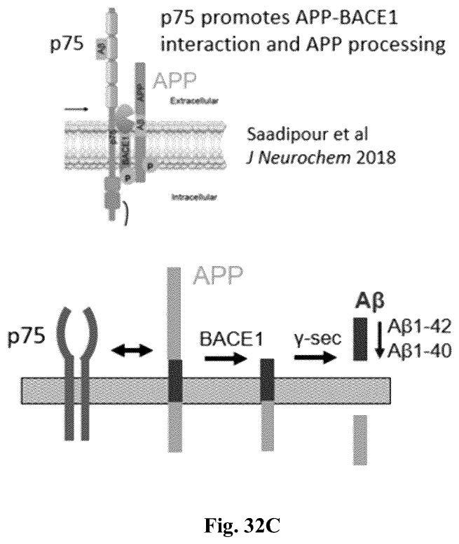

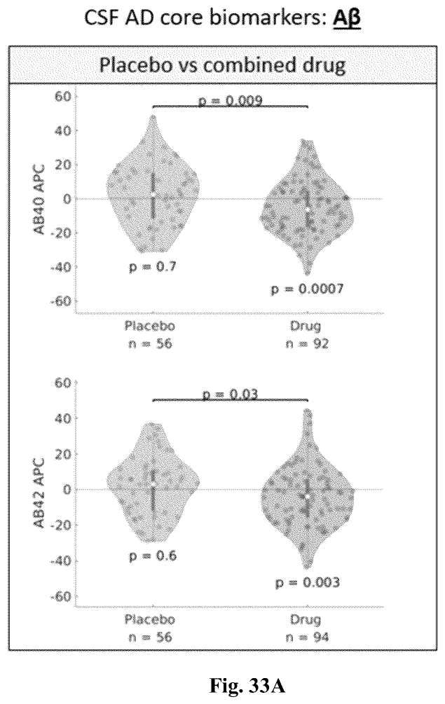

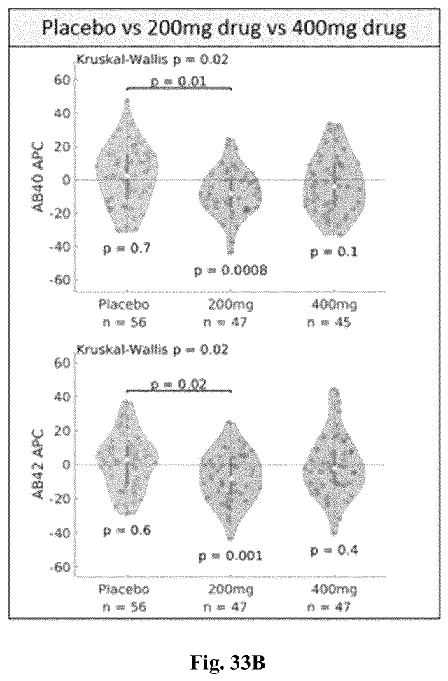

In the placebo group, Ab 42 went up slightly (not significant, p=0.6) but in the drug group it went down by a significant amount (p=0.003) as shown in FIGS. 32A-32C. The difference between placebo and drug is significant (p=0.03). The ability of drug to decrease CSF Ab 42 levels was not expected. The decrease however is consistent with the drug engaging its p75 receptor target. The 2018 study on the right found that if p75 is removed away from the amyloid precursor protein (APP) which it normally associates with, the ability of BACE1 to process APP into Ab42 is decreased. Since LM11A-31 causes endocytosis of the p75 receptor, it would be expected to move it away from APP when it engages p75 and thus LM11A-31 when engaging its p75 receptor target would be expected to lead to decreased CSF Ab levels. This shows, for the first time, that small molecule modulation of p75 is able to affect Ab 42 and Ab 40 levels. Another class of AD treatment, the BACE inhibitors, also has a goal of inhibiting APP processing to Ab 42. Thus LM11A-31 achieves a desired effect in humans that is similar to the BACE inhibitors. The drug also promotes increased volume of the dentate gyrus which is a part of the hippocampus and is vulnerable to degeneration in Alzheimer's and other neurodegenerative diseases. Drug dose analysis of Aβ 42 and 40 in CSF in subjects treated with placebo, 200 mg bid or 400 mg bid of LM11A-31 reveals that both doses demonstrate a trend for reduction of Aβ 42 and 40 levels relative to placebo with the 200 mg dose reaching statistical significance (FIGS. 33A and 33B). The 200 mg dose also leads to a significant reduction in Aβ 42 and 40 relative to pre-treatment baseline values. These findings support target engagement of LM111A-31-BHS with the p75 receptor given that p75 mediates the production of Aβ 42 and 40 from the amyloid protein precursor (FIGS. 33A and 33B). As described herein, the dataset indicated as “a combined drug” are derived from corresponding analyses that combine both datasets indicated as “200 mg” and “400 mg” for the purpose of statistical tests.

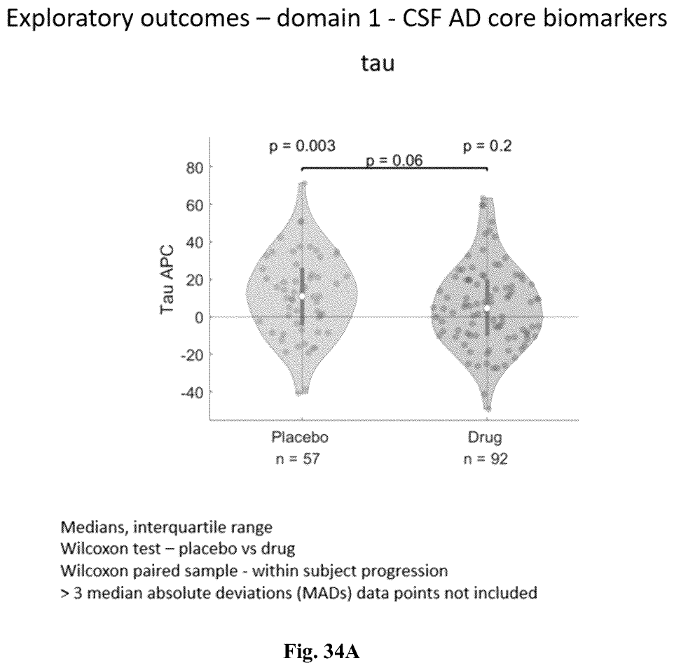

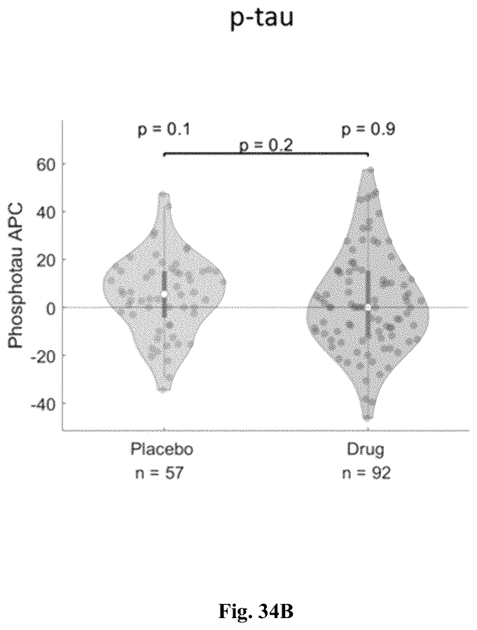

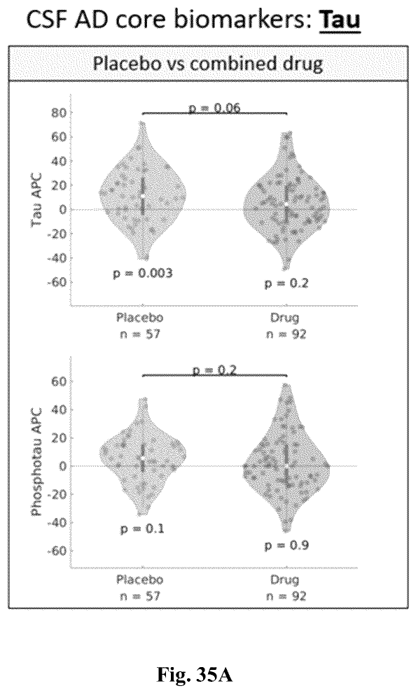

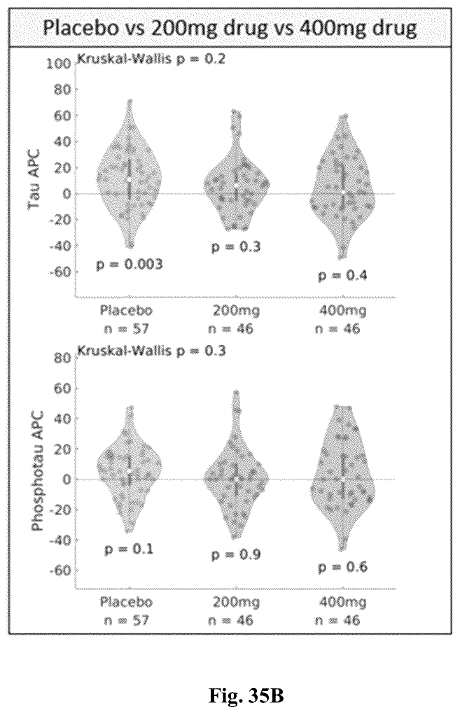

In the placebo group, CSF tau levels increase significantly (p=0.003). In the drug group, this increase is no longer detected at a significant level (p=0.2). When compared to placebo, the drug has a near-significant effect compared to placebo in decreasing the elevation of tau that is occurring over the 6 month treatment period (p=0.06). For p-tau, there is a trend for the drug to inhibit the trend to rise in p-tau in the placebo group (FIGS. 34A and 334B). Drug dose analysis of total tau and phospho-tau CSF levels in CSF in subjects treated with placebo, 200 mg bid or 400 mg bid of LM11A-31 reveals trends for lowering of total tau reduction, especially at the 400 mg dose (FIGS. 35A and 35B). Analysis of p-tau indicates similar trends for reduction at both doses. Total tau analysis demonstrates a significant increase in the placebo group which is no longer present at the 200 and 400 mg doses. The total tau findings suggest the possibility that LM11A-31-BHS, at over the 200 and 400 mg dose range, and possibly at lower and higher doses, reduces neuronal injury and the p-tau findings indicated a drug effect in reducing pathological tau phosphorylation events occurring in Alzheimer's disease (FIGS. 35A and 35B).

Exploratory Outcomes for Domain 1 Related with CSF Pre-Synaptic Biomarkers

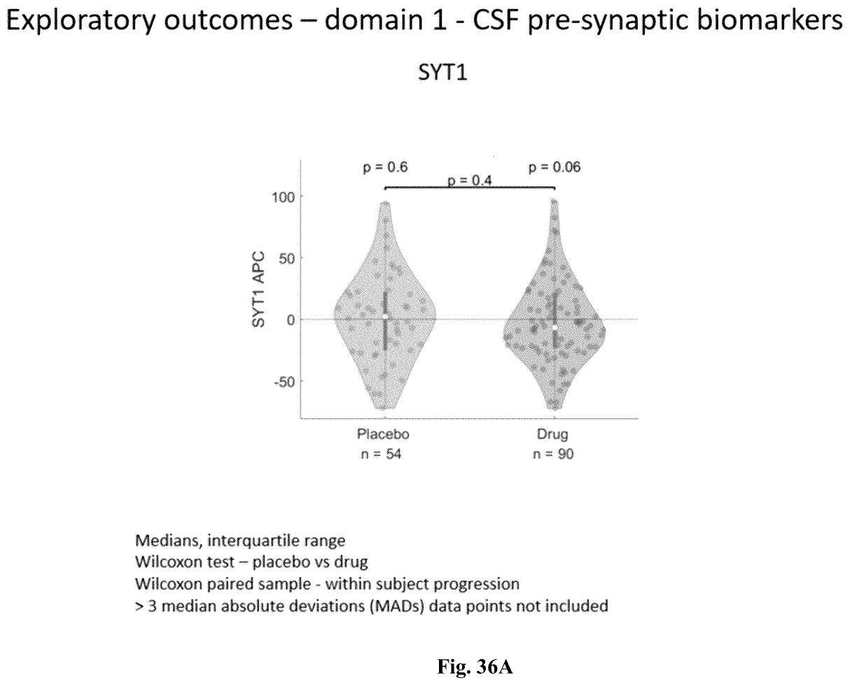

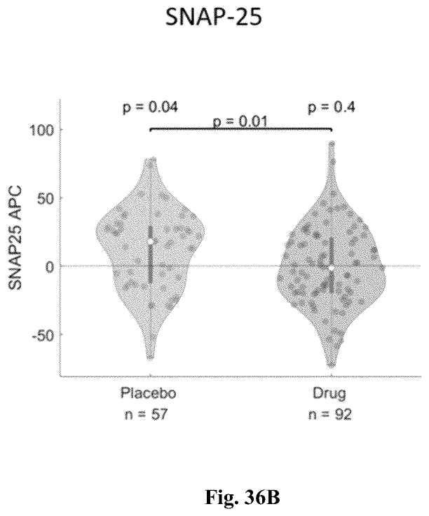

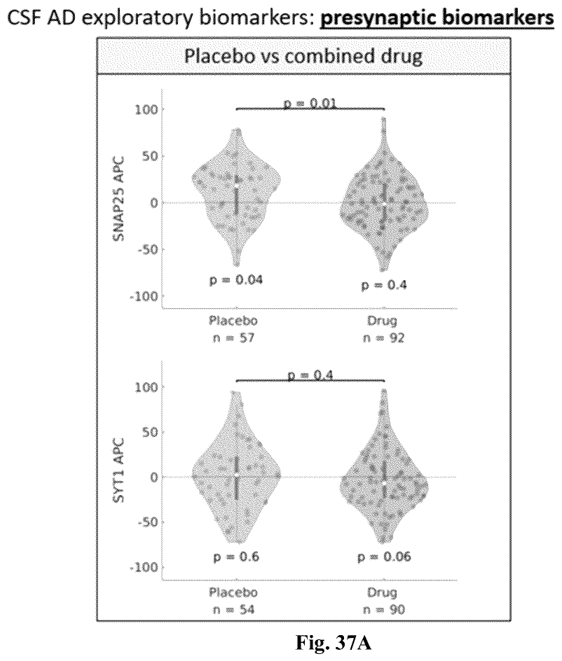

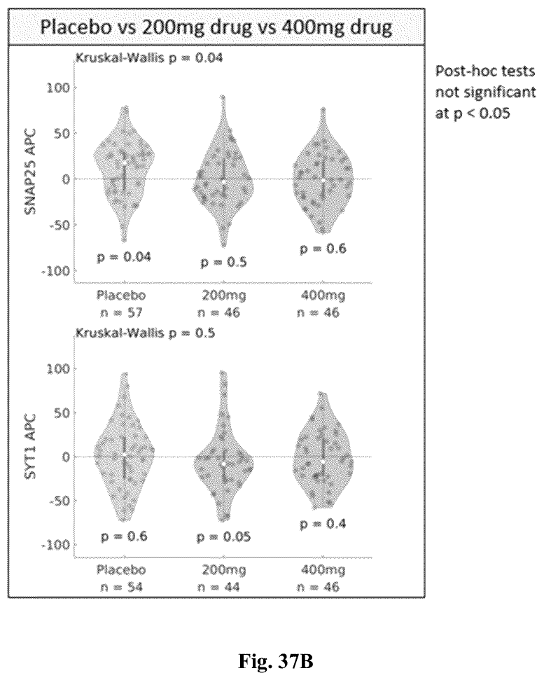

In the placebo group the levels of SYT1 remain about the same during the 6 months of the study (FIGS. 36A and 36B). There is a trend for a decrease in the drug group (p=0.06) indicating a beneficial effect on pre-synaptic components. For the SNAP-25 pre-synaptic marker, there was a significant elevation during the 6-month period indicating significant degeneration of pre-synaptic elements. In the drug group no increase was detected indicating a significant (p=0.01) drug effect in blocking progression of degeneration in presynaptic elements. To our knowledge this is the first demonstration of a therapy preventing degeneration of pre-synaptic terminals as indicated by this pre-synaptic marker. In FIGS. 37A and 37B, the SNAP-25 kruskall wallis test detects a difference in the 3 overall group distributions, but the post hoc testing with Dunn's test does not reach significance. Drug dose analysis of the SNAP-25 and SYT1 pre-synaptic markers in CSF in subjects treated with placebo, 200 mg bid or 400 mg bid of LM11A-31 reveals trends for prevention of SNAP-25 increases that are found in the placebo group with a significant main effect of drug dose which appears to be driven by lowering of SNAP-25 at the 200 and 400 mg doses (FIGS. 37A and 37B). SNAP-25 analysis demonstrates a significant increase in the placebo group which is no longer present at the 200 and 400 mg doses. Analysis of SYT1 levels demonstrates only nominal reductions below baseline at both doses. The SNAP-25 data indicates that drug treatment prevents degeneration of pre-synaptic components of synapses.

Exploratory Outcomes for Domain 1 Related with CSF Post-Synaptic Biomarkers

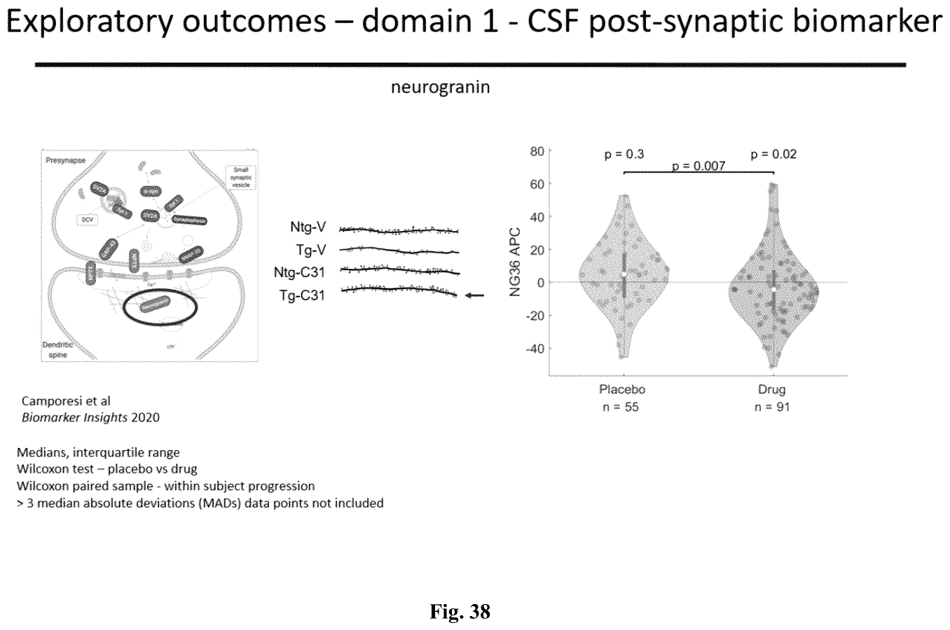

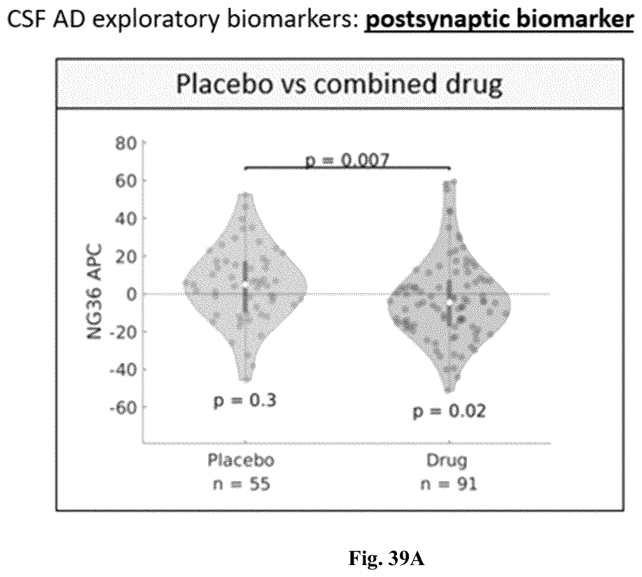

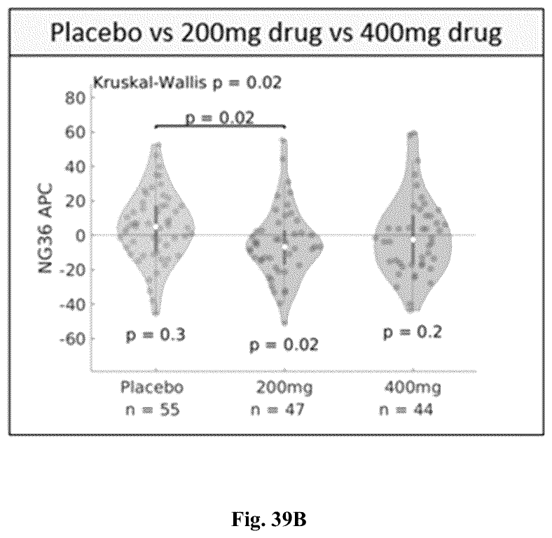

Neurogranin is present in post synaptic elements known as dendritic spines. In the placebo group there was a trend to increase in neurogranin levels during the 6-months (FIG. 38). Notably, in the drug group, there was a significant decrease (p=0.02) in neurogranin levels indicating not only a block of increased degeneration, but a reversal of spine degeneration as was found in the mouse studies (middle panel). Drug dose analysis of the neurogranin-36 post-synaptic marker in CSF in subjects treated with placebo, 200 mg bid or 400 mg bid of LM11A-31 reveals significant lowering of NG-36 at the 200 mg dose and a trend for reduction at the 400 mg dose (FIGS. 39A and 39B). The 200 mg dose also demonstrates a significant lowering of NG-36 relative to the pre-treatment baseline value. This NG-36 data indicates that drug treatment prevents degeneration of post-synaptic components of synapses.

Exploratory Outcomes for Domain 1 Related with CSF Glial Biomarkers

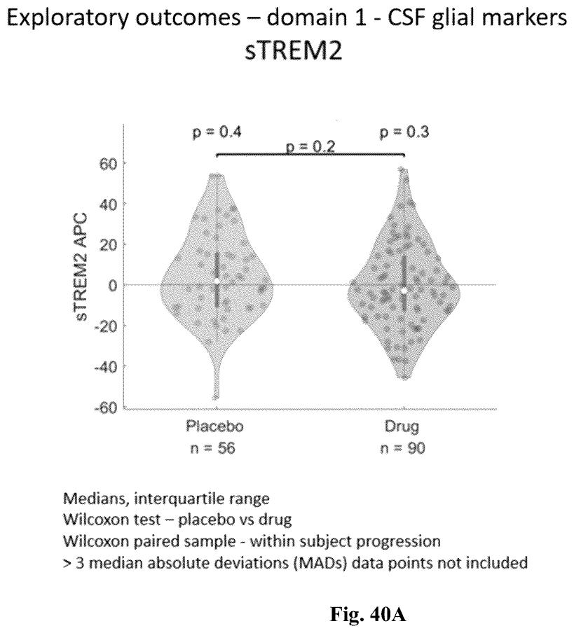

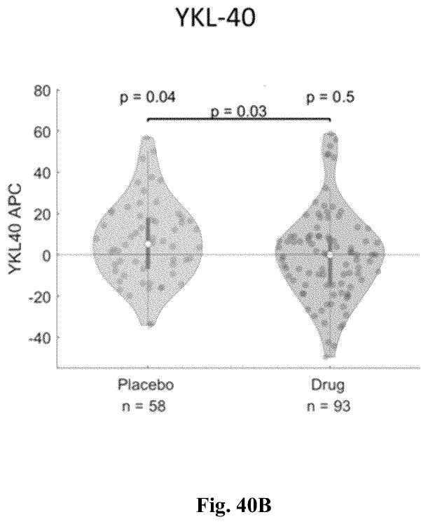

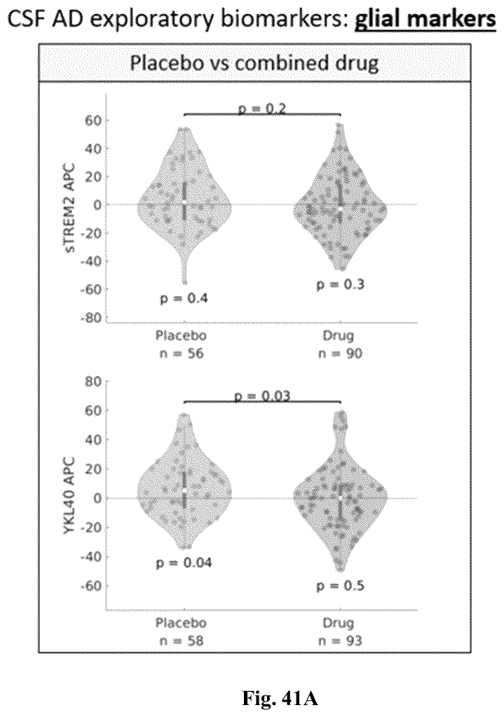

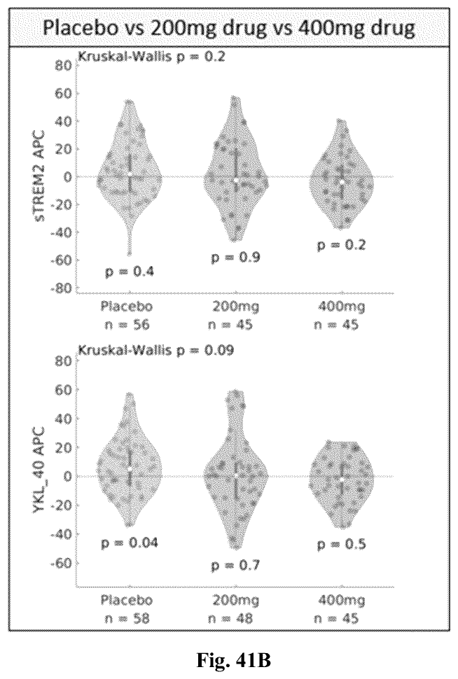

CSF levels of sTREM2 did not change during the 6-month period and no drug effect was detected (FIGS. 40A and 40B). Levels of CSF YKL-40 increase significantly (p=0.04) indicating increased gliosis and neuroinflammation during this period. This increase was not detected in the drug group and the difference between placebo and drug groups was significant (p=0.03) indicting that treatment reduces progression of neuroinflammatory processes. FIGS. 4A and 41B1 show drug dose analyses of the sTREM2 and YKL40 glial activation markers. sTREM2 analysis reveals minimal change for sTREM2 in the placebo group with a nominal reduction at the 200 and 400 mg doses. YKL40 analysis demonstrates a significant increase in the placebo group which is no longer present at the 200 and 400 mg doses.

### Exploratory Outcomes for Domain 2 Using Structural MRI

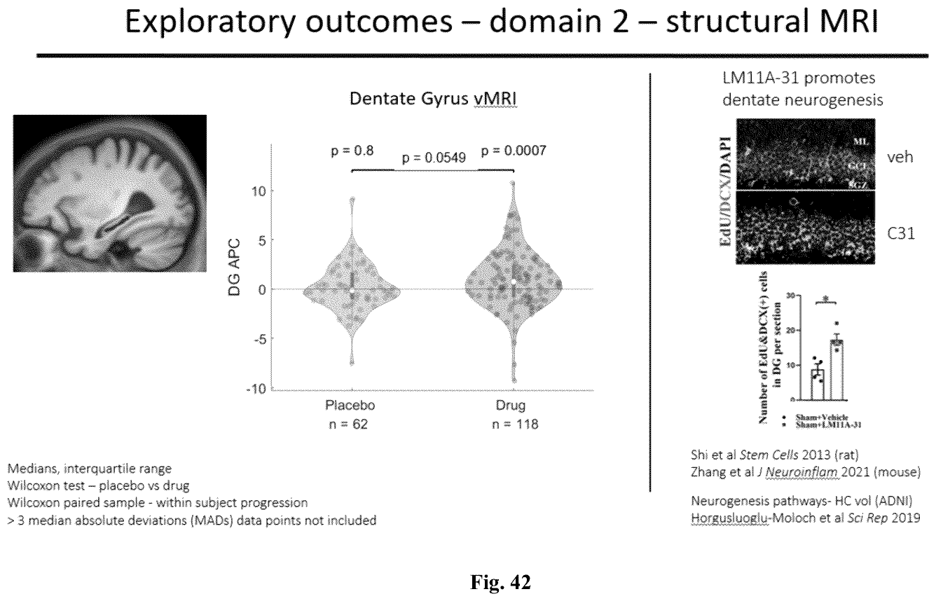

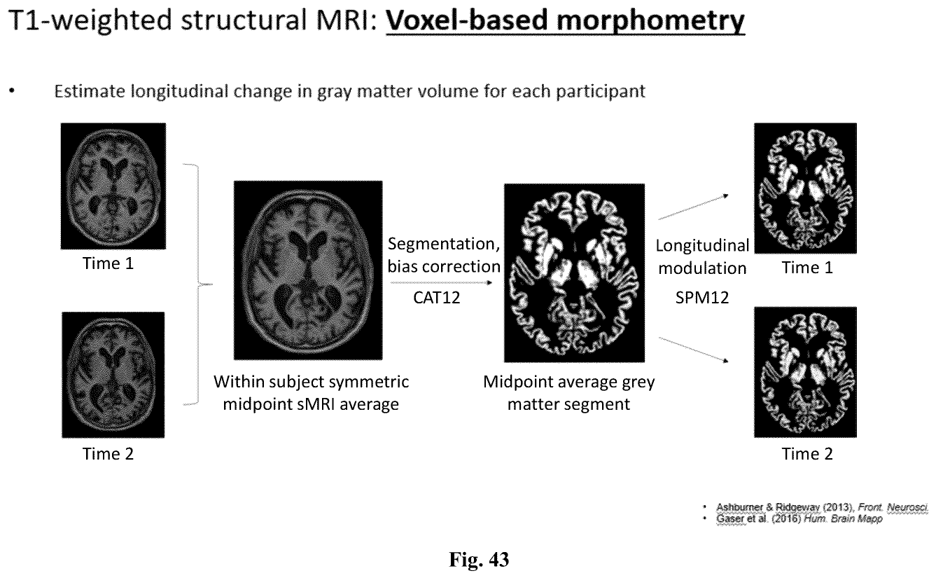

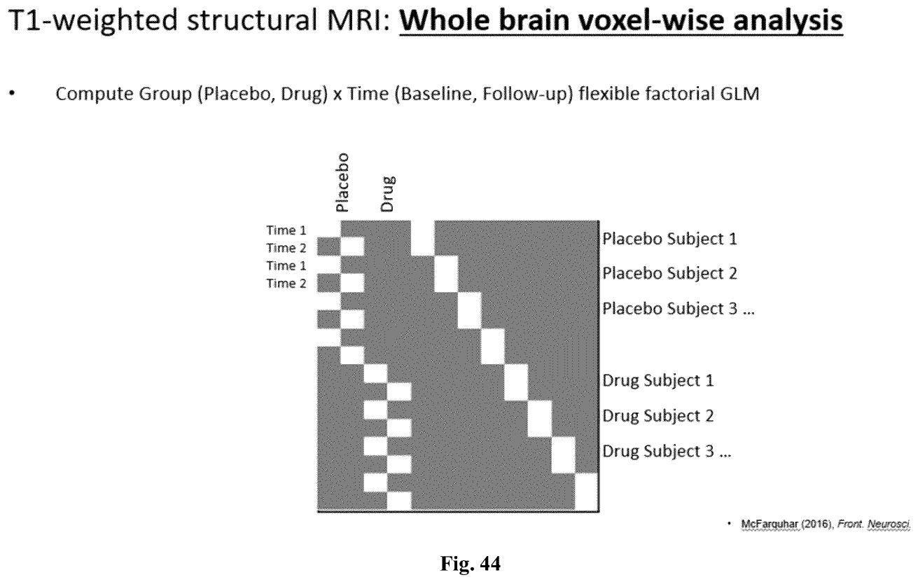

There was no change detected in HC volume during the 6-month period (FIG. 42). Notably, there was a significant increase in HC volume in the drug group (p=0.006). This finding is consistent with the known effects in preclinical models of the drug in promoting hippocampal neurogenesis and promoting neurite growth and complexity. The dentate gyrus is the part of the hippocampus in which neurogenesis occurs (formation of new neurons on the adult). Promotion of neurogenesis is a candidate mechanism for leading to increased volume of the dentate gyrus. One cannot directly measure neurogenesis in humans. The drug was shown to lead to a significant increase in the volume of the dentate gyrus in humans and this effect is consistent with demonstrations that the p75 receptor is present on neuroprogenitor cels and that the drug promotes dentate neurogenesis in mice and rat studies.

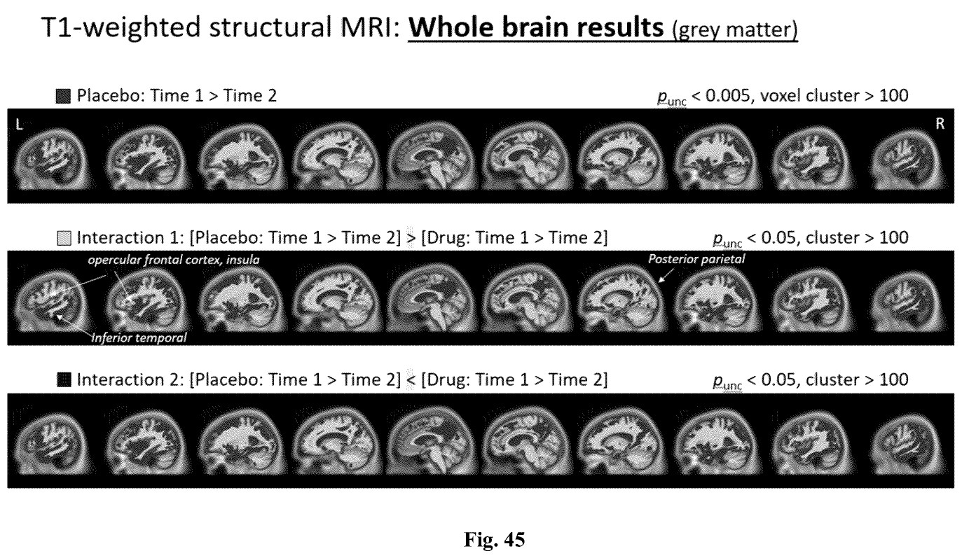

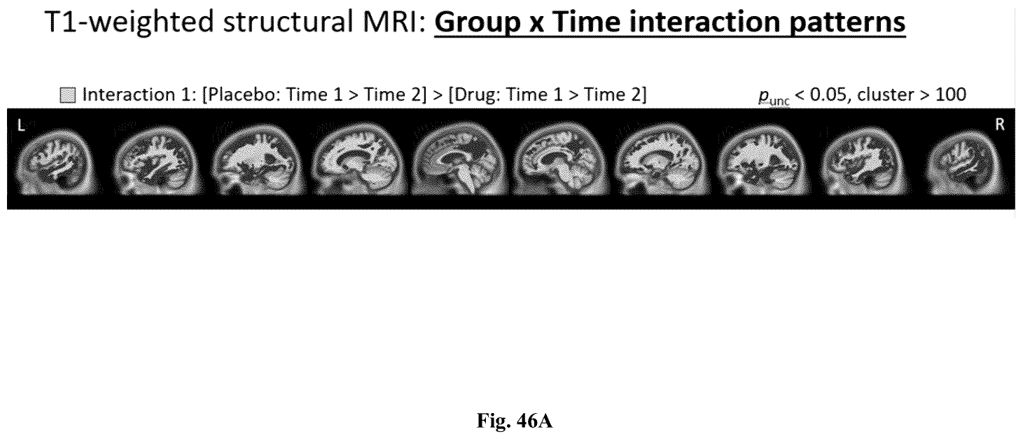

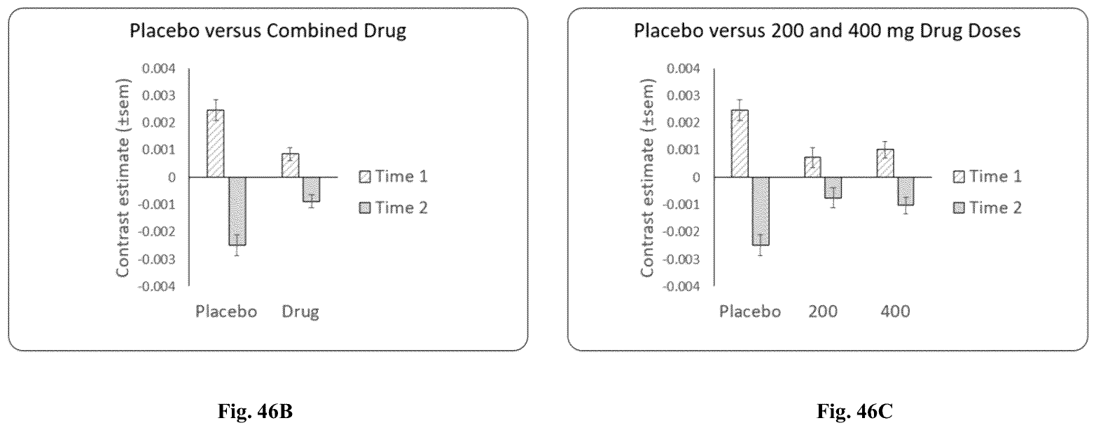

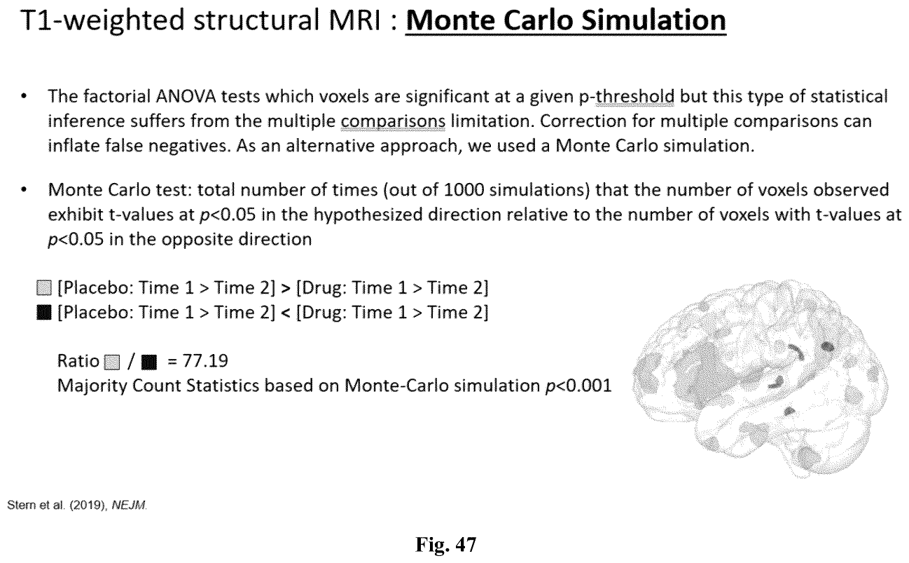

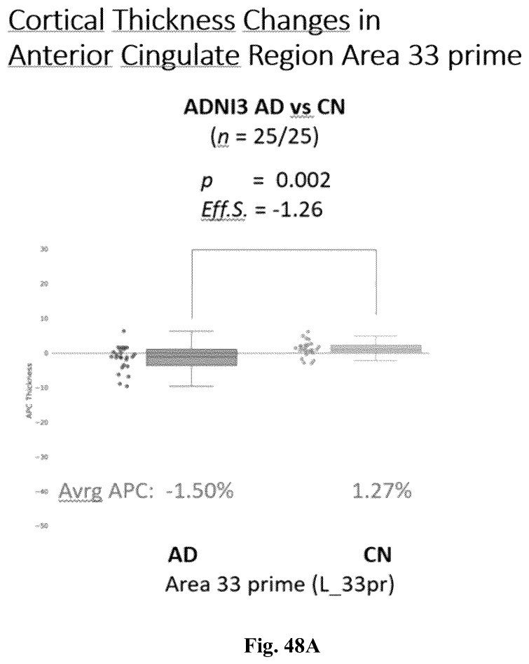

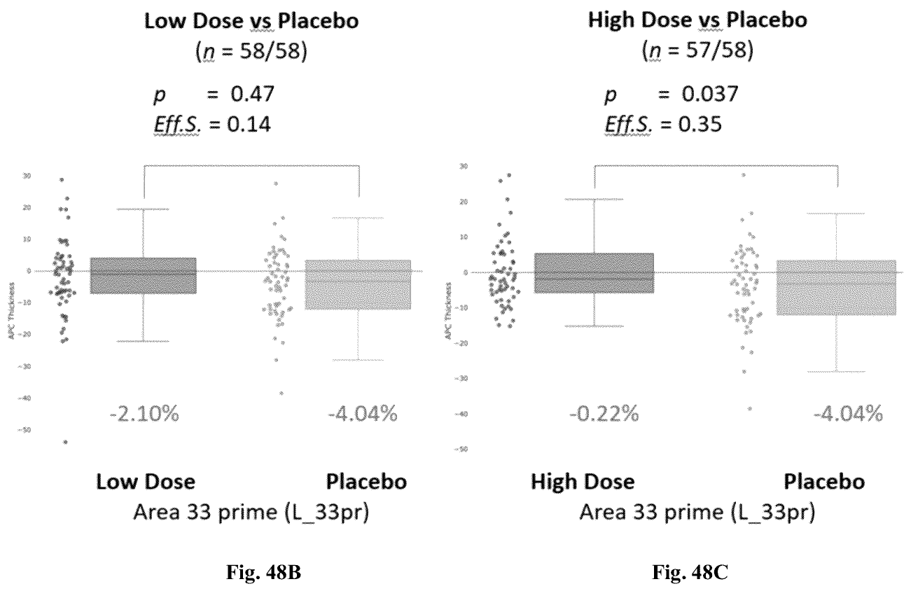

FIG. 45 shows whole brain results of T1-weighted structural MRI. In FIG. 45, upper row indicates (red) regions demonstrating loss of volume in the placebo treatment group between baseline (Time 1) and post-treatment (Time 2). Middle row demonstrates regions of loss of volume as shown in the upper row (red) and areas in green indicate regions in which volume loss in the placebo group was significantly greater than that found in drug-treated subjects. For this analysis, the drug treatment group contains subject treated with either 200 or 400 mg doses. The lower row demonstrates regions of loss of volume as shown in the upper row (red) and areas in blue indicate regions in which volume loss in the placebo group was significantly less than that found in drug-treated subjects. Overall, these findings indicate multiple areas within the degenerating region that demonstrate slowed degeneration in the drug group and rare areas demonstrating increased degeneration in the drug group. FIGS. 46A-46C show group x time interaction patterns using T1-weighted structural MRI. Contrast estimate plots show the magnitude and direction of the interaction effects observed in the flexible factorial GLM. Hence, the bars in green show the magnitude of volume change over Time at each level of treatment Group, averaged over all voxels in the green overlay of the map above, i.e. voxels which show a pattern of [Placebo: Time 1>Time 2] loss of volume greater than drug loss of volume [Drug: Time 1>Time 2] at a p<0.05 and cluster size >100. Dose analysis indicates a significant effect at both the 200 and 400 mg doses in slowing loss of volume. FIG. 47 shows T1-weighted structural MRI, Monte Carlo Simulation (MCS). The ratio and p-value output of the MCS is derived from the total number of times (out of 1000 simulations) that the number of voxels observed in the input map (at a given p and k threshold) exhibit t-values in the hypothesized direction (slowing loss of volume with drug treatment) relative to the number of voxels with t-values in the opposite direction. Therefore, if the actual observed ratio of t-values is close to equivalent in the input map at a given p and k threshold, the MCS ratio and p-value will tend to support the null hypothesis of no drug effect. If the actual observed ratio is skewed heavily in favor of one direction over the other (as demonstrated here with a ratio of 77.19), the MCS ratio and p-value will tend to reject the null hypothesis. These findings demonstrate a significant drug effect in slowing loss of brain volume and is consistent the GLM results. FIGS. 48A-48C show a drug effect on improving cortical thickness or decreasing gyrification in a broad sense as both are indicators of cortical atrophy/degeneration. Increased thickness is similar to increased volume and means less degeneration. Here increased gyrification means increased atrophy.

### Exploratory Outcomes for Domain 3: Cognition

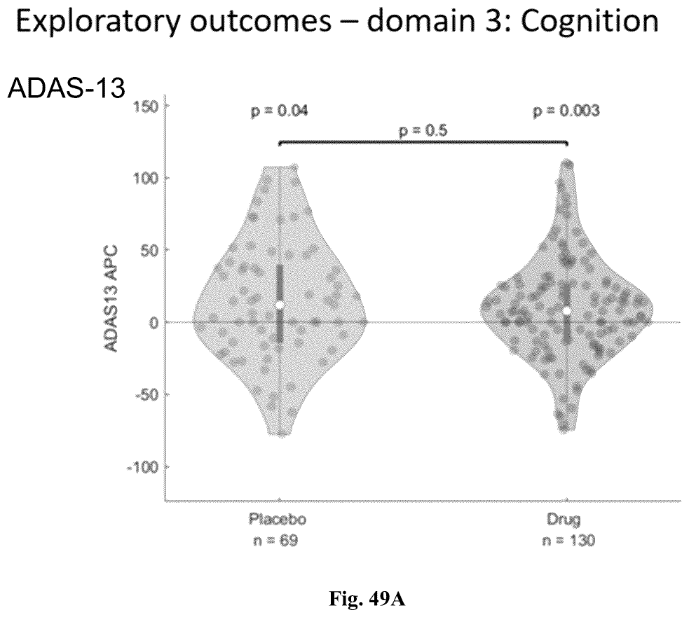

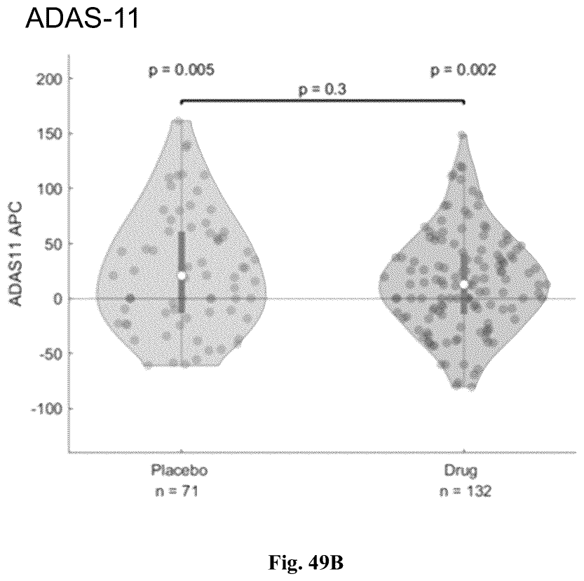

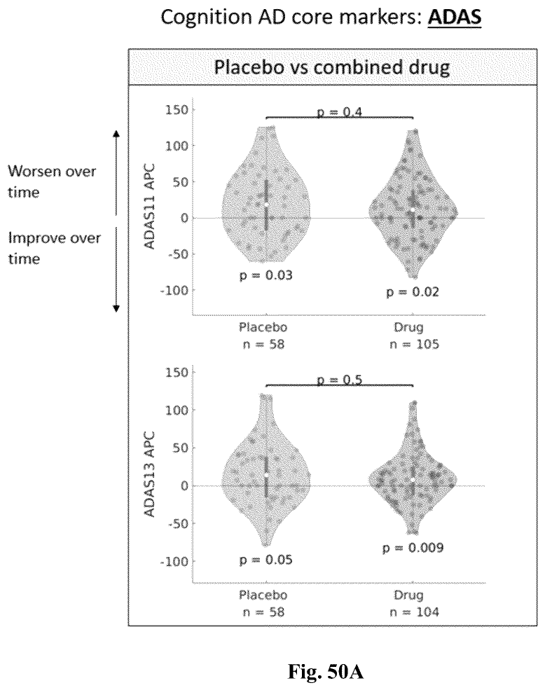

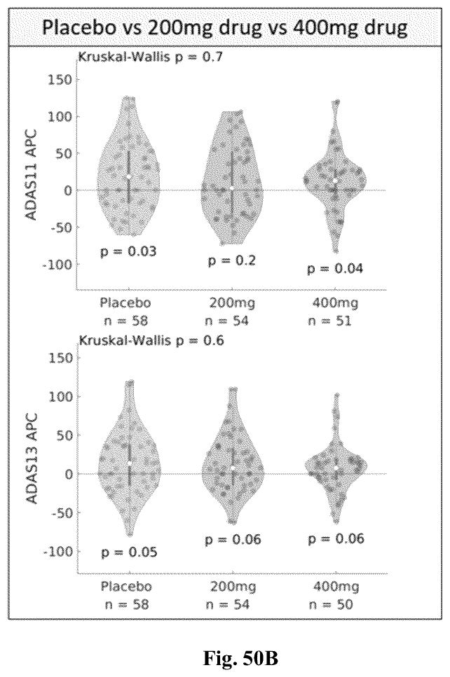

There is significant worsening of cognitive function but both the ADAS-13 and -11 scores (FIGS. 49A and 49B). The drug is associated with a trend in slowing the worsening, but this effect does not reach statistical significance in the study which as an exploratory trial was not powered sufficiently to derive statistically significant cognitive data. FIGS. 50A and 50B show drug dose analysis of ADAS-cog-1 1 and -13 cognition scores in subjects treated with placebo, 200 mg bid or 400 mg bid of LM11A-31. In the placebo group, there was a significant increase in −11 and −13 scores indicating a decline in cognition. Analysis for ADAS-cog-11 measures indicate a trend for decreased worsening of cognitive scores at both doses with a loss of the significant increase in score in the 200 mg group. Analysis for ADAS-cog-13 demonstrates a nominal decrease in the increase in score found in the placebo group in the 200 and 400 mg groups.

### Exploratory Outcomes for Domain 4: FDG-PET

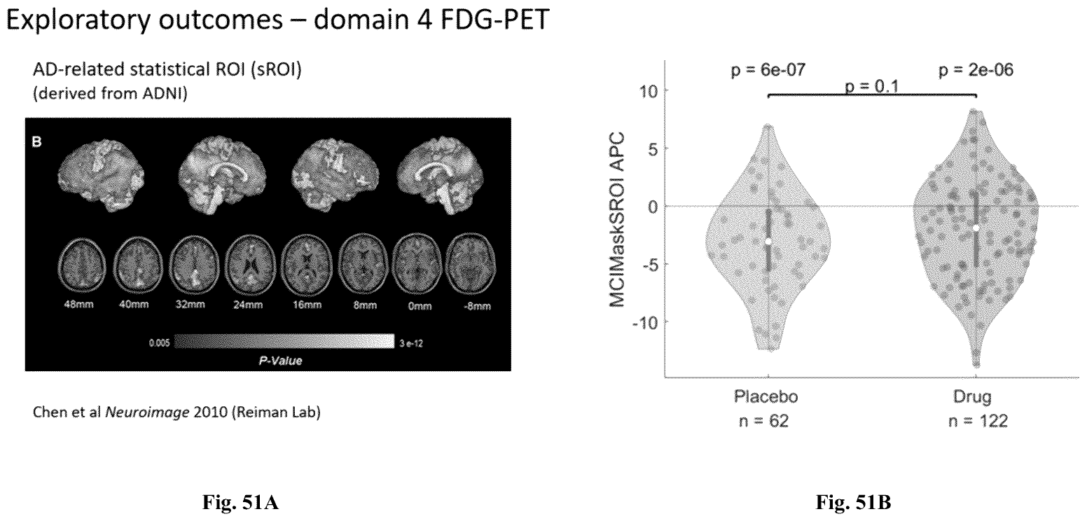

FDG-PET using statistical ROI measures derived from ADNI as described in the Chen et al. paper. This measure indicted significant decline in the placebo group with a trend to less decline over the 6 month period in the drug group (FIGS. 51A and 51B).

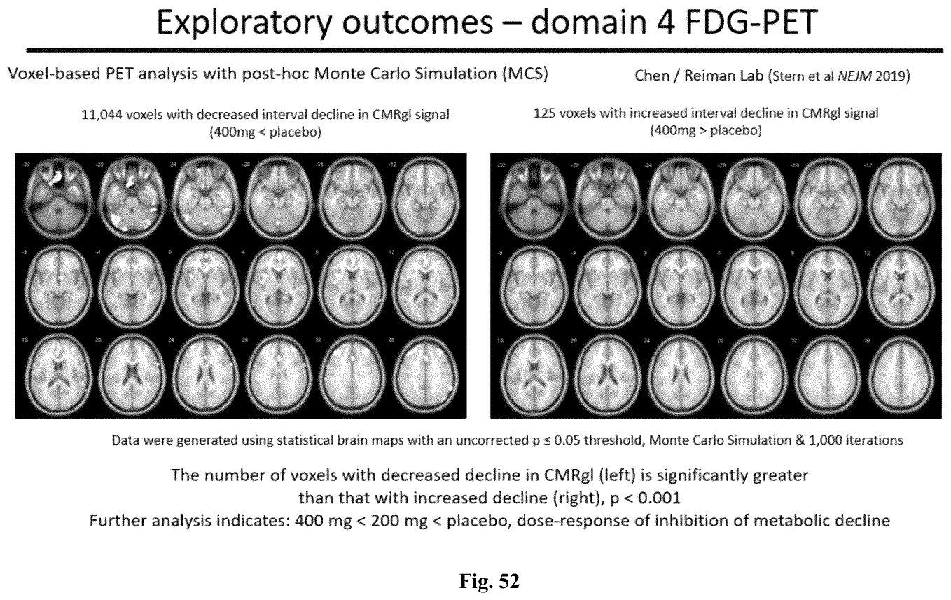

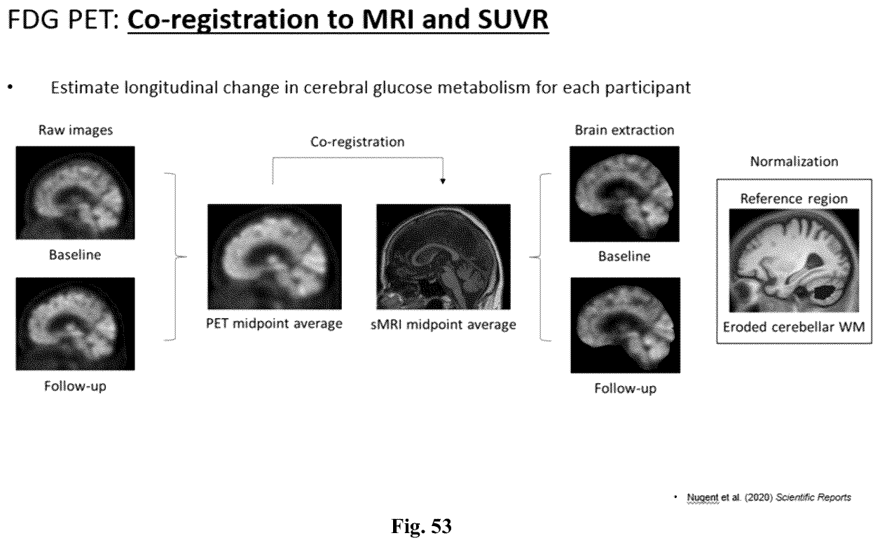

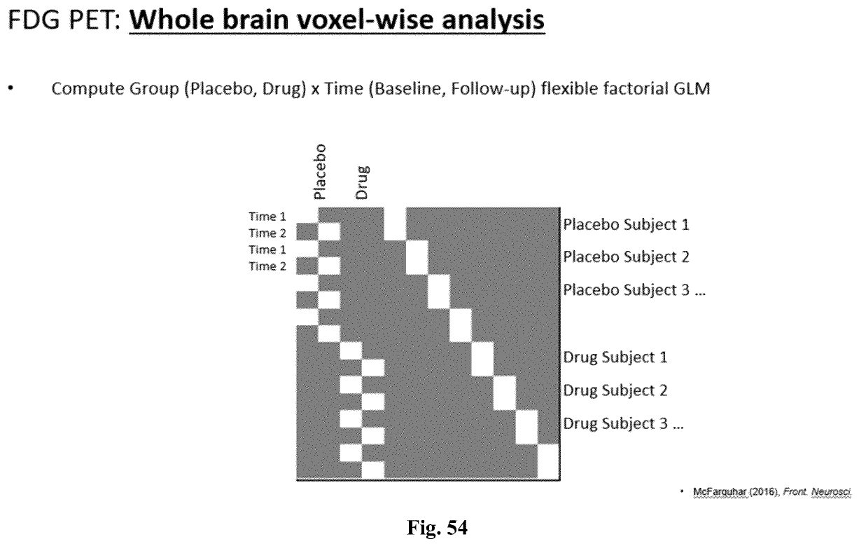

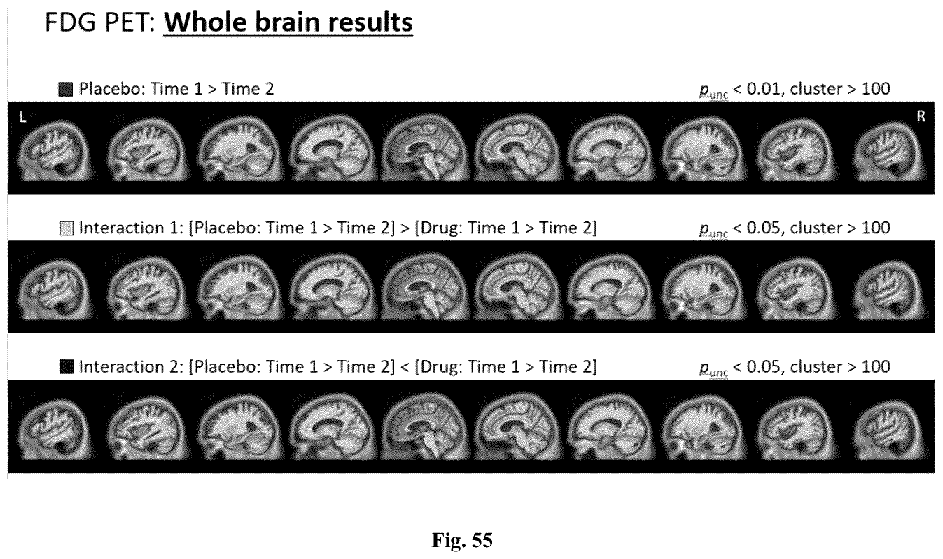

FDG-PET using the MCS (Monte Carlo Simulation) analysis approach is described in Stem et al. The number of voxels showed less decline (left) in metabolic function (a surrogate measure of synaptic function) in the drug while the placebo group was significantly much higher than the number of voxels showing increased decline (right) (FIG. 52). Areas showing less decline are highlighted in yellow. It is notable that these areas are relevant to areas that degeneration in AD. The effect at the 400 mg dose is higher than that at the 200 mg dose which is higher than that found in placebo (no effect found in placebo). Thus, a dose response effect is demonstrated. FIG. 53 shows three-dimensional co-registration of structural MRI and FDG-PET as a strategy for analysis of FDG-PET metabolic signal (SUVR—Standardized update value ratio) changes over time. This method makes possible analysis of FDG-PET signal in a common three-dimensional sampling space with the MRI analysis for each subject thereby enhancing accuracy of FDG-PET analysis and allowing the combination of the two modalities for further analyses and the assessment of the spatial relationship between structural and functional degeneration over time. FIG. 54 shows FDG-PET using whole brain voxel-wise analysis. It demonstrates flexible factorial generalized linear model (GLM) analysis matrix for brain voxel-wise FDG-PET metabolic analysis. FIG. 55 shows FDG-PET whole brain SUVR analysis. In FIG. 55, upper row indicates (red) regions demonstrating loss of SUVR signal in the placebo treatment group between baseline (Time 1) and post-treatment (Time 2). Middle row demonstrates regions of loss of SUVR signal as shown in the upper row (red) and areas in green indicate regions in which SUVR signal loss in the placebo group was significantly greater than that found in drug-treated subjects. For this analysis, the drug treatment group contains subjects treated with either 200 or 400 mg doses. The lower row demonstrates regions of loss of SUVR signal as shown in the upper row (red) and areas in blue indicate regions in which SUVR signal loss in the placebo group was significantly less than that found in drug-treated subjects. Overall, these findings indicate multiple areas within the degenerating region that demonstrate slowed degeneration in the drug group and rare areas demonstrating increased degeneration in the drug group.

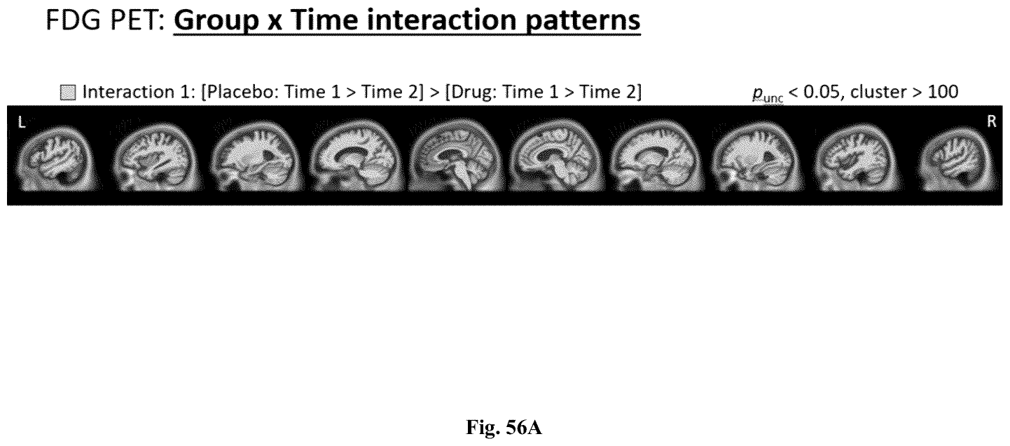

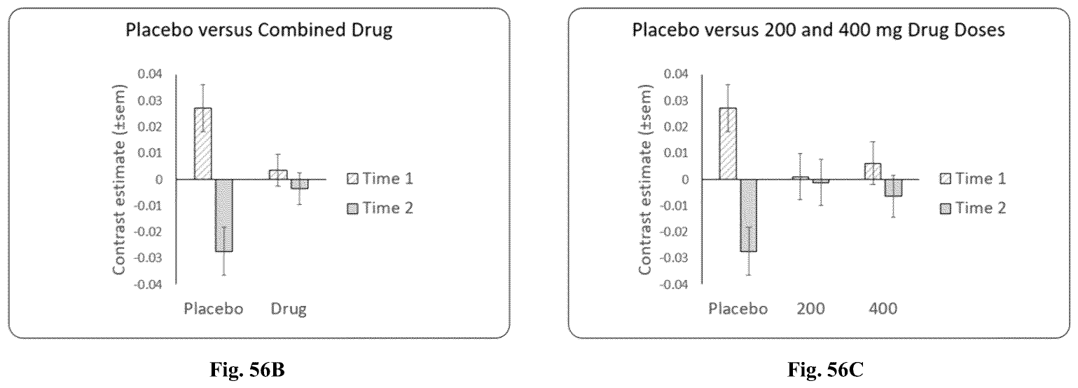

FIGS. 56A-56C show FDG PET using group x time interaction patterns. In FIGS. 56A-56C, contrast estimate plots show the magnitude and direction of the interaction effects observed in the flexible factorial GLM. Hence, the bars in green show the magnitude of SUVR change over Time at each level of treatment Group, averaged over all voxels in the green overlay of the map above, i.e. voxels which show a pattern of [Placebo: Time 1>Time 2] loss of SUVR signal greater than drug loss of SUVR signal [Drug: Time 1>Time 2] at a p<0.05 and cluster size >100. Dose analysis indicates a significant effect at both the 200 and 400 mg doses in slowing loss of SUVR signal.

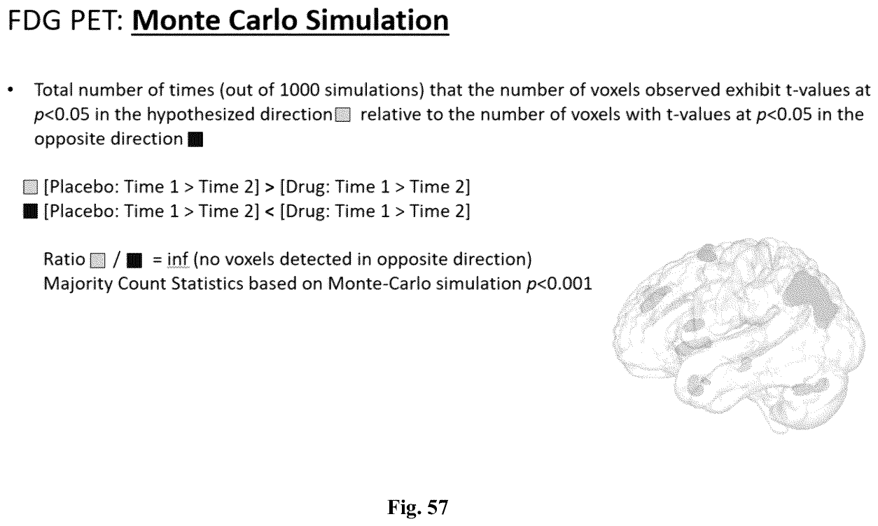

FIG. 57 shows FDG PET using MCS analysis. In FIG. 57, the ratio and p-value output of the MCS is derived from the total number of times (out of 1000 simulations) that the number of voxels observed in the input map (at a given p and k threshold) exhibit t-values in the hypothesized direction (slowing loss of SUVR signal with drug treatment) relative to the number of voxels with t-values in the opposite direction. Therefore, if the actual observed ratio of t-values is close to equivalent in the input map at a given p and k threshold, the MCS ratio and p-value will tend to support the null hypothesis of no drug effect. If the actual observed ratio is skewed heavily in favor of one direction over the other (as demonstrated here with a ratio of infinity), the MCS ratio and p-value will tend to reject the null hypothesis. These findings demonstrate a significant drug effect in slowing loss of brain metabolic function and is consistent the GLM results.

In general, for the range of biomarkers, effects at both doses are similar in many cases and in some cases, effects are greater at the higher dose. Given the smaller numbers available for analysis in the separate dose groups, many of these analyses do not reach statistical significance. The optimal design for a phase 3 trial would continue to include both doses when considering: dose-related side effects; likely efficacy at both doses; and some loss of statistical power for a given number of total subjects by including both doses versus one dose.

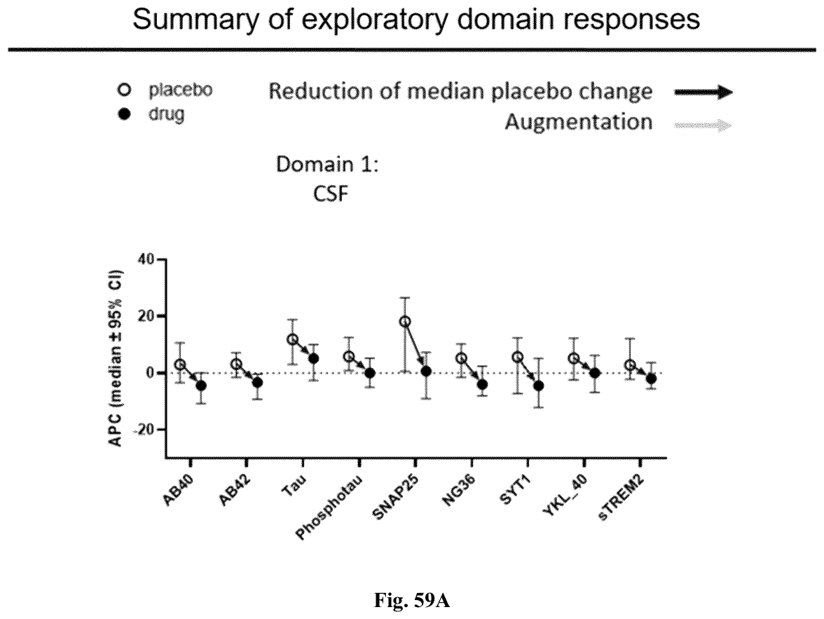

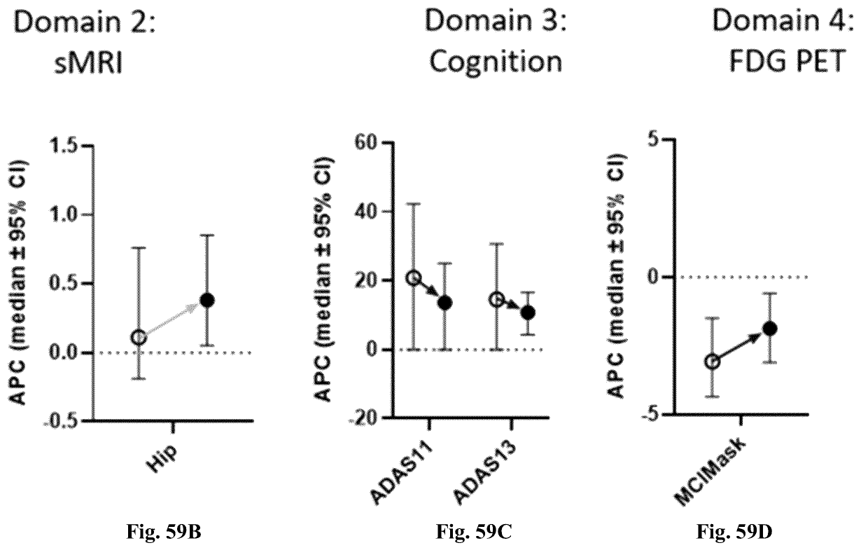

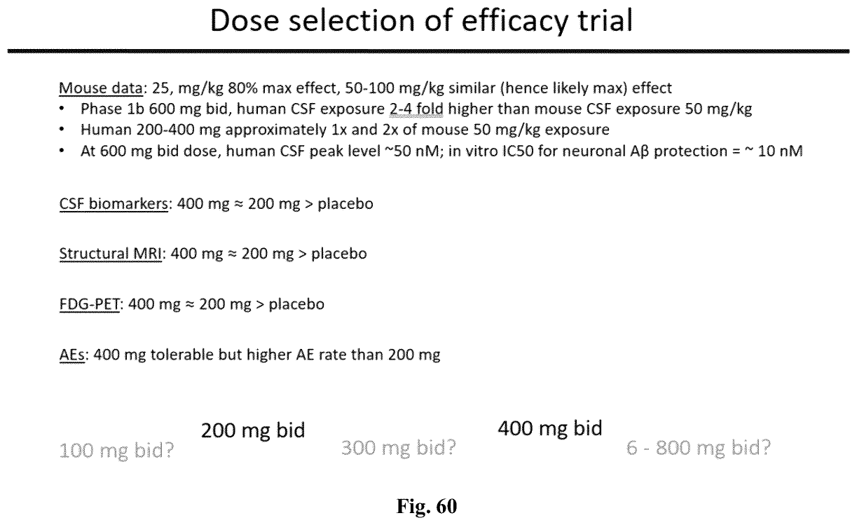

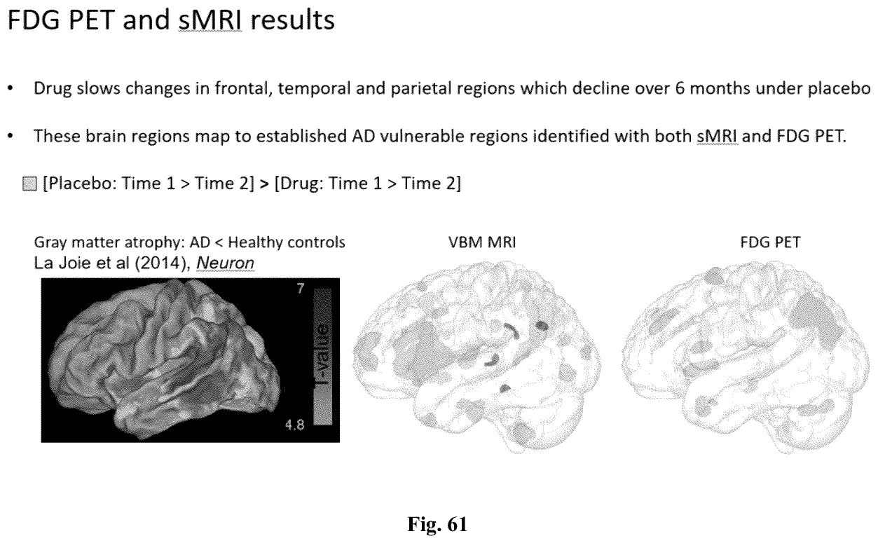

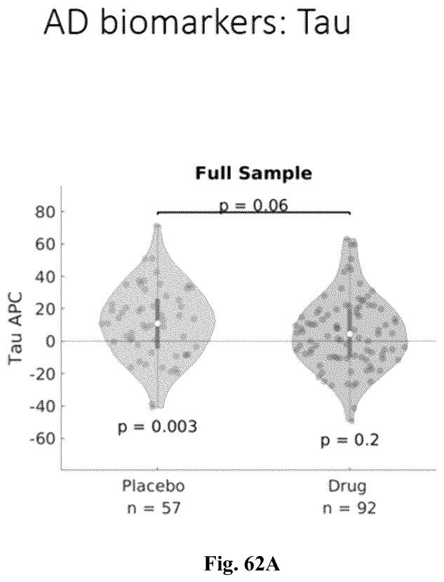

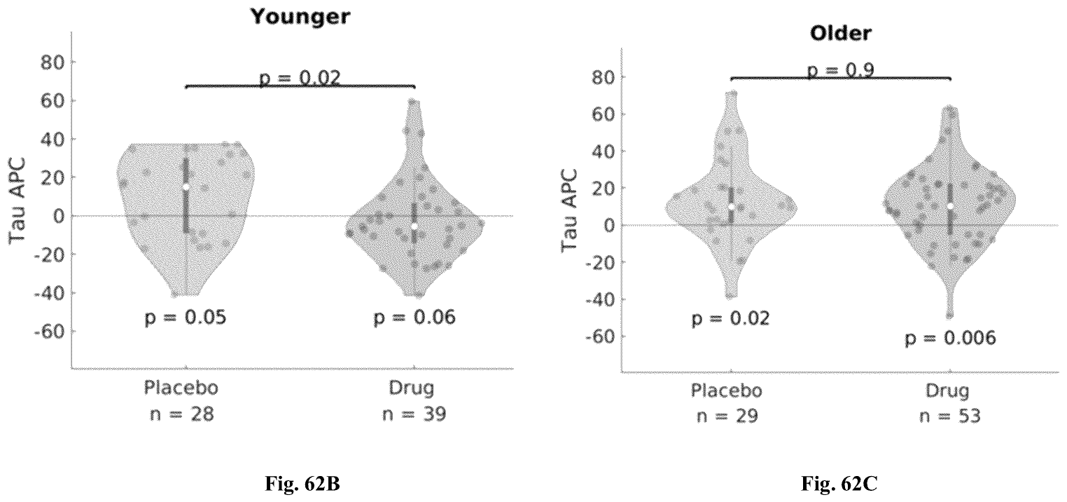

FIGS. 59A-59D show summary of the placebo progression measured by the measures in the 4 outcome domains and the effect and trend of the drug. The relatively remarkable consistency of the trend across all 4 outcome domains of the drug to correct measures of progression occurring in the placebo group is strongly suggestive of an underlying effect of the drug to slow degeneration in AD.

### Example 15. Two Phase 1 Trials (Phase 1 and Phase 1b), Randomized, Double-Blind, Placebo Controlled Dose Study

The phase 1 study was placebo controlled and double blinded and included 52 subjects in SAD studies and 20 subjects in MAD studies. The summary conclusions are listed below:
LM11A-31 and its metabolite exhibited linear pharmacokinetics after single oral doses as exposure was proportional to dose over a range from 100-900 mg of free base.
Upon multiple dosing, LM11A-31 and its aminoethyl morpholine metabolite appear to reach steady state by approximately 5 days post dose.
In elderly adults, there is an apparent food effect for both parent and metabolite, with fed subjects having a higher LM11A-31 parent exposure than fasted subjects.
Preliminary results suggest a gender difference in LM11A-31 exposure, with female subjects having a greater exposure than males when receiving unit doses.
LM11A-31-BHS, administered as single doses of 100 mg, 300 mg, 600 mg, and 900 mg free base, and as multiple doses of 300 BID and 600 mg free base QD, was generally safe and well tolerated.
Following treatment with a single dose of LM11A-31-BHS, 1 of 40 subjects experienced a total of 1 TEAE (postural dizziness considered possibly related to study drug).
During administration of multiple doses of LM11A-31-BHS, 7 of 16 subjects experienced a total of 8 TEAEs, 6 of which were considered unrelated to study drug. The remaining 2 events were considered possibly (flatulence) or remotely (nocturia) related to study drug.
There were no deaths, SAEs, severe TEAEs, and no subjects were discontinued because of an AE. All TEAEs were mild.
No clinically significant laboratory results, physical examination findings, vital signs, ECG findings, or positive scores on C—SSRS assessments were observed.

A phase 1b studied was conducted in 10 elderly normal subjects for the purpose of obtaining plasma and CSF coordinated drug levels and to further assess safety at a dose of 600 mg bid over a 10-day period. The study was double-blinded and placebo controlled with 8 subjects receiving drug. Results are summarized below:
During the study there were no clinically relevant alterations in the physical examination, vital signs and ECG recordings.
Minimal changes observed in haematological and biochemistry parameters, had neither clinical relevance nor relationship with the study drug.
Results support the safety and tolerability of LM11A-31LM11A-31-HBS in a repeated dose schedule of 600 mg BID up to ten days of treatment.
Pharmacokinetics of 600 mg BID of LM11A-31 during 10 days has shown to be—linear.

### Example 16. Age Effect on CSF Biomarkers Examined in Phase IIa

FIGS. A-62C show age effect analysis for CSF total tau biomarker(s). In the placebo group, during the 6-month trial period, total tau protein concentration increased in the CSF as measured in terms of annual percent change (APC) from baseline to the post-treatment measure. This increase indicated ongoing neural degeneration in the placebo group. In the drug group, there was less increase, indicating a slowing of neural degeneration. Within the younger group of study subjects (<72 years of age), the drug effect in slowing or inhibiting the tau increase was greater than that found in the older group (>=72 years of age). These findings indicate that while the drug has effects on slowing neural degeneration across the entire age group, its effects are particularly notable in the younger group.

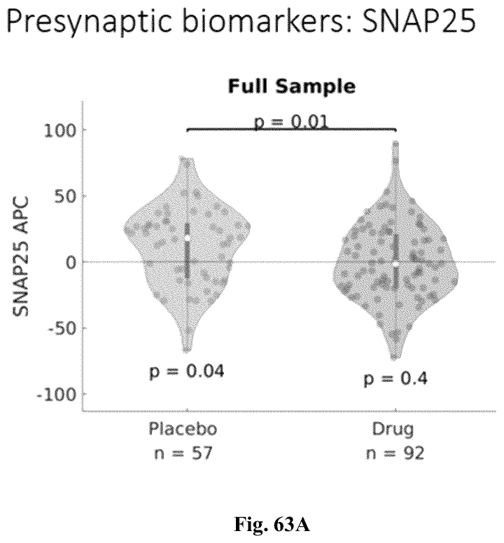

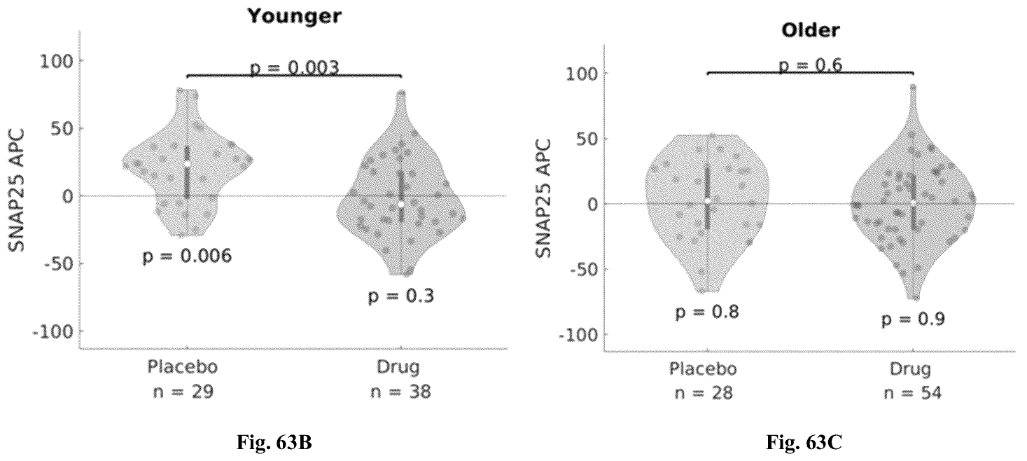

FIGS. 63A-63C show age effect analysis for CSF SNAP-25 presynaptic biomarker. In the placebo group, during the 6-month trial period, SNAP-25 protein concentration increased in the CSF as measured in terms of annual percent change (APC) from baseline to the post-treatment measure. This increase indicated ongoing pre-synaptic degeneration in the placebo group. In the drug group, there was less increase, indicating a slowing of presynaptic degeneration. Within the younger group of study subjects (<72 years of age), the drug effect in slowing or inhibiting the SNAP-25 increase was greater than that found in the older group (>=72 years of age); in part related to the lack of SNAP-25 increase in the older placebo group. These findings indicate that while the drug has effects on slowing pre-synaptic degeneration across the entire age group, its effects are particularly notable in the younger group.

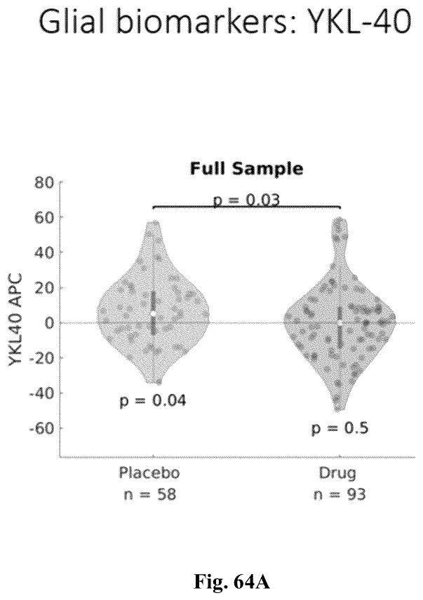

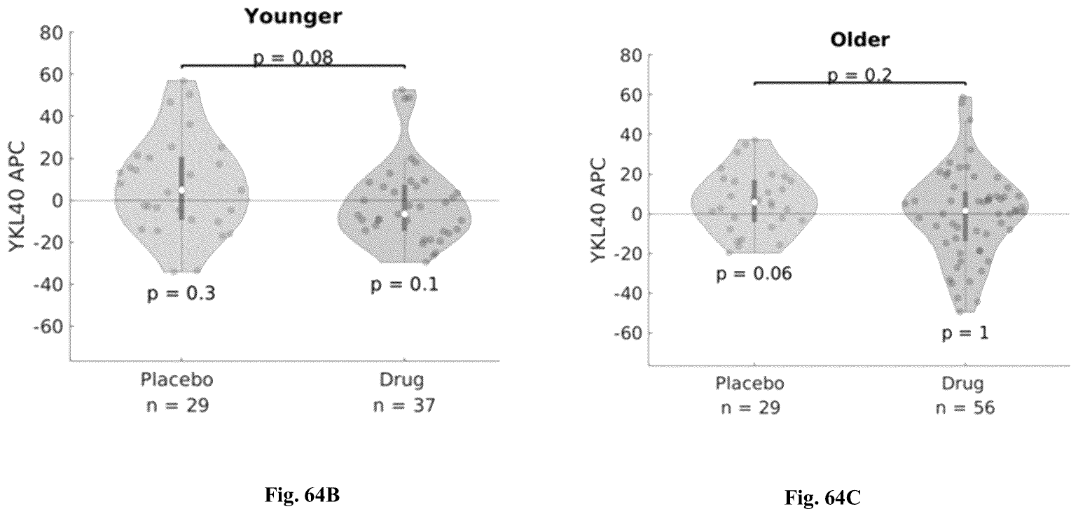

FIGS. 64A-64C show age effect analysis for the YKL-40 glial biomarker. In the placebo group, during the 6-month trial period, YKL-40 protein concentration increases in the CSF as measured in terms of annual percent change (APC) from baseline to the post-treatment measure. This increase indicates ongoing pathological glial response in the placebo group. In the drug group, there is less increase, indicating a slowing of pathological glial response. Within the younger group of study subjects (<72 years of age), the drug effect in slowing or inhibiting the pathological glial response is greater than that found in the older group (>=72 years of age). These findings indicate that while the drug has effects on slowing pre-synaptic degeneration across the entire age group, its effects are particularly notable in the younger group.

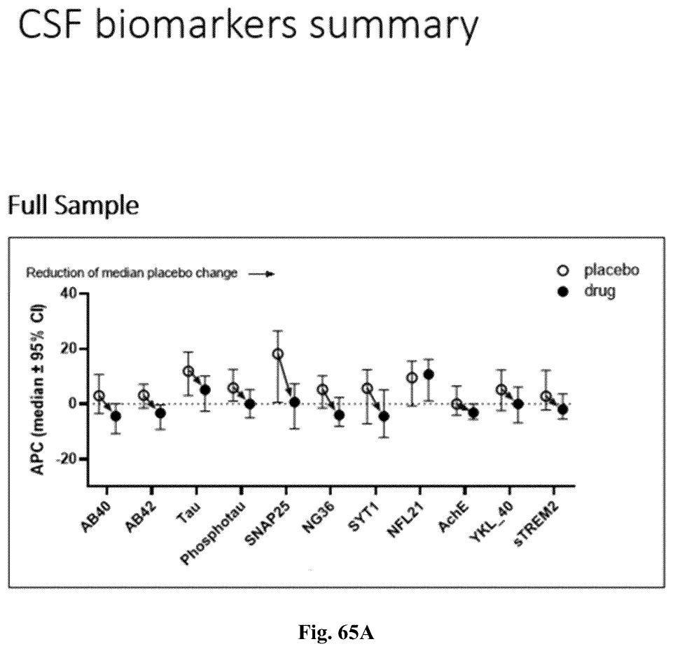

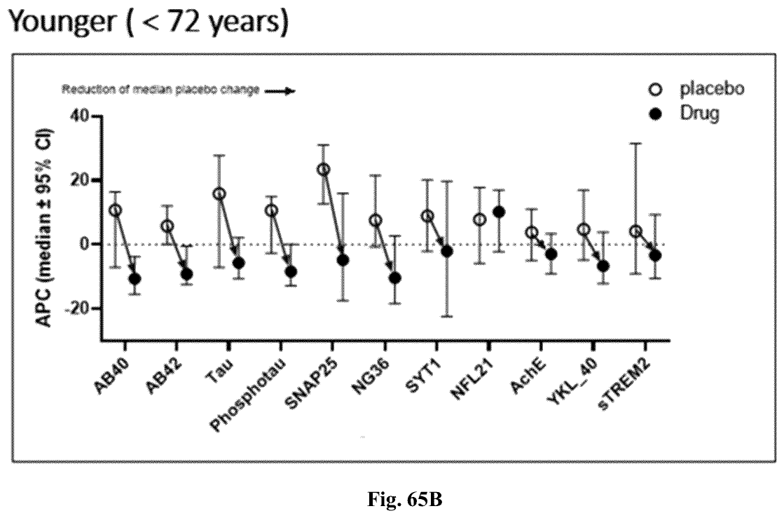

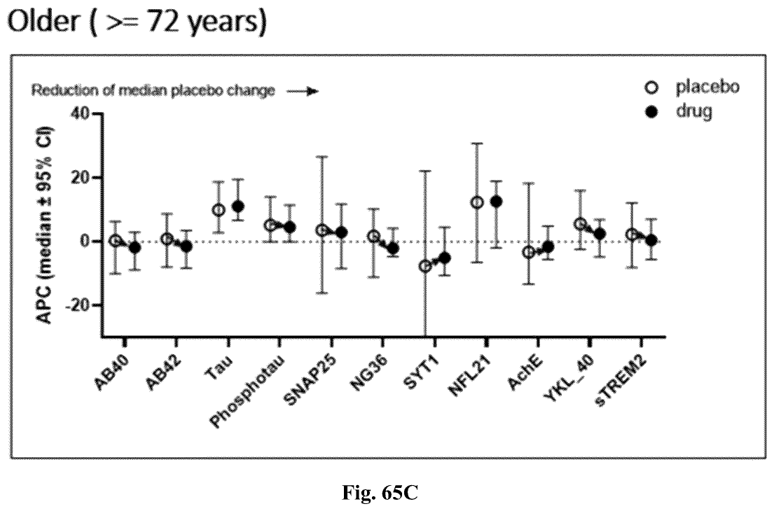

FIGS. 65A-65C show summary of all CSF biomarkers examined in the phase 2a study. The CSF biomarkers included Ab 40, Ab 42, tau, phosphotau, SNAP 25, NG36, SYT1, NFL21, AchE, YKL_40, and sTREM2. For biomarkers that did not show statistically significant increases in the placebo group, there was nevertheless a consistent pattern across the biomarker group such that when a trend for increase was present, the drug treatment group demonstrated less of a trend for increase and the drug effect was in general greater in the younger versus older group.

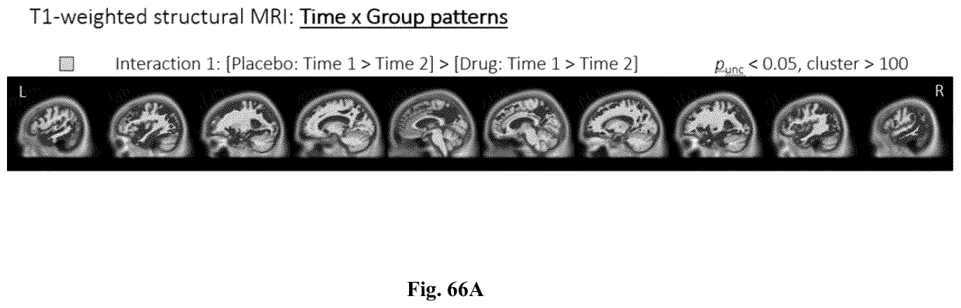

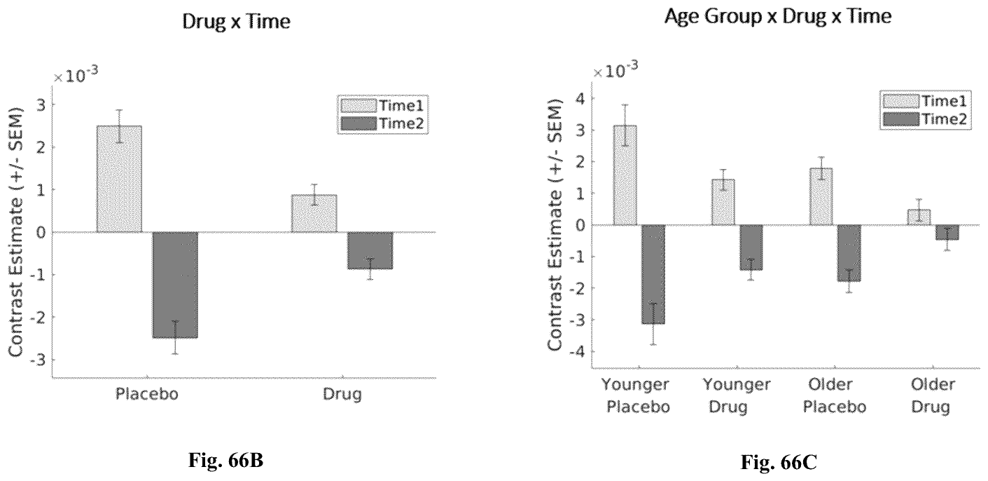

FIGS. 66A-66C show T1-weighted structural MRI analysis. Red regions demonstrated loss of volume in the placebo treatment group between baseline (Time 1) and post-treatment (Time 2) at a p<0.05 and cluster size >100. Green regions indicated regions within the red degeneration region, in which volume loss in the placebo group was significantly greater than that found in drug-treated subjects. These findings indicate multiple areas within the degenerating region that demonstrate slowed degeneration in the drug group. Contrast estimate plots show the magnitude and direction of the interaction effects observed in the flexible factorial general linear model (GLM). Bars in green show the magnitude of volume change over Time at each level of treatment Group, averaged over all voxels in the green overlay of the map above, i.e. voxels which show a pattern of [Placebo: Time 1>Time 2] loss of volume greater than drug loss of volume [Drug: Time 1>Time 2] at a p<0.05 and cluster size >100. Age analysis indicates a significant drug effect in both age groups (younger, <72 years of age; older, >=72 years of age).

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A method for modulating one or more neurodegeneration biomarkers or indicators in a subject in need thereof, the method comprising:
administering to said subject an effective amount of a p75^{NTR} receptor modulator or a pharmaceutically acceptable salt thereof to provide one or more of the following in said subject:
(i) an amyloid beta (AD) level (e.g., in a bodily fluid) that is lower than a corresponding reference;
(ii) a change (e.g., increase) or a rate of change (e.g., a rate of increase) in an Aβ level (e.g., in a bodily fluid) over a duration that is lower than a corresponding reference over the same duration;
(iii) a change (e.g., increase) or a rate of change (e.g., a rate of increase) in a tau (t) level (e.g., in a bodily fluid) over a duration that is lower than a corresponding reference over the same duration;
(iv) a change (e.g., increase) or a rate of change (e.g., a rate of increase) in a presynaptic biomarker level (e.g., in a bodily fluid) over a duration that is lower than a corresponding reference over the same duration;
(v) a change (e.g., increase) or a rate of change (e.g., a rate of increase) in a postsynaptic biomarker level (e.g., in a bodily fluid) over a duration that is lower than a corresponding reference over the same duration;
(vi) a change (e.g., increase) or a rate of change (e.g., a rate of increase) in a glial marker level (e.g., in a bodily fluid) over a duration that is lower than a corresponding reference over the same duration;
(vii) a rate of volume change of a brain region over a duration that is lower than a corresponding reference rate (e.g., in an untreated control) over the same duration as determined by magnetic resonance imaging (MRI) imaging;
(viii) a brain glucose metabolism (¹⁸F-FDG-PET) rate that is lower than a corresponding reference rate (e.g., in an untreated control); and
(ix) a change in Alzheimer's Disease Assessment Scale-Cognitive (ADAS-Cog) score over a duration that is less than a corresponding change (e.g., in an untreated control) over the same duration.

2. A method for modulating one or more neurodegeneration biomarkers or indicators in a subject in need thereof, the method comprising: administering to said subject an effective amount of a p75^{NTR} receptor modulator or a pharmaceutically acceptable salt thereof to provide an amyloid beta (Aβ) level (e.g., in a bodily fluid) in said subject that is lower than a corresponding reference.

3. A method for modulating one or more neurodegeneration biomarkers or indicators in a subject in need thereof, the method comprising: administering to said subject an effective amount of a p75^{NTR} receptor modulator or a pharmaceutically acceptable salt thereof to provide a change (e.g., increase) or a rate of change (e.g., a rate of increase) in an Aβ level (e.g., in a bodily fluid) over a duration that is lower than a corresponding reference over the same duration.

4. The method of claim 3, wherein the duration is at least about 5 days, at least about 10 days, at least about 15 days, at least about 20 days, at least about 25 days, at least about 30 days, at least about 1 month, at least about 2 months, at least about 3 months, at least about 4 months, at least about 5 months, or at least about 6 months.

5. The method of claim 2, wherein said Aβ is Aβ40 or Aβ42.

6. The method of claim 5, wherein said Aβ is Aβ40.

7. The method of claim 5, wherein said Aβ is Aβ42.

8. The method of claim 2, wherein said bodily fluid is a cerebrospinal spinal fluid, blood or plasma.

9. The method of claim 2, wherein the method provides an Aβ level in said bodily fluid in said subject that is lower by at least about 1%, at least about 2%, at least about 3%, at least about 4%, or at least about 5% than said corresponding reference.

10. The method of claim 2, wherein said corresponding reference is a corresponding pretreatment level in said bodily fluid.

11. The method of claim 2, wherein said corresponding reference is determined from an untreated control.

12. A method for modulating one or more neurodegeneration biomarkers or indicators in a subject in need thereof, the method comprising: administering to said subject an effective amount of a p75^{NTR} receptor modulator or a pharmaceutically acceptable salt thereof to provide a change (e.g., increase) or a rate of change (e.g., a rate of increase) in a tau (t) level (e.g., in a bodily fluid) over a duration that is lower than a corresponding reference over the same duration.

13. The method of claim 12, wherein the duration is at least about 5 days, at least about 10 days, at least about 15 days, at least about 20 days, at least about 25 days, at least about 30 days, at least about 1 month, at least about 2 months, at least about 3 months, at least about 4 months, at least about 5 months, or at least about 6 months.

14. The method of claim 12, wherein said tau comprises phosphorylated tau.

15. The method of claim 12, wherein said bodily fluid is a cerebrospinal spinal fluid, blood or plasma.

16. The method of claim 12, wherein said method provides a tau (t) level in said bodily fluid in said subject that as a rate of increase lower by at least about 1, at least about 2, at least about 3, at least about 4, or at least about 5 annual percent change (APC)) than said corresponding reference.

17. The method of claim 12, wherein said corresponding reference is a corresponding pretreatment level in said bodily fluid.

18. The method of claim 12, wherein said corresponding reference is determined from an untreated control.

19. A method for modulating one or more neurodegeneration biomarkers or indicators in a subject in need thereof, the method comprising: administering to said subject an effective amount of a p75^{NTR} receptor modulator or a pharmaceutically acceptable salt thereof to provide a change (e.g., increase) or a rate of change (e.g., a rate of increase) in a presynaptic biomarker level (e.g., in a bodily fluid) over a duration that is lower than a corresponding reference over the same duration.

20. The method of claim 19, wherein the duration is at least about 5 days, at least about 10 days, at least about 15 days, at least about 20 days, at least about 25 days, at least about 30 days, at least about 1 month, at least about 2 months, at least about 3 months, at least about 4 months, at least about 5 months, or at least about 6 months.

21.-98. (canceled)
